SEQUENCE LISTING (5 sequences)

SEQ ID NO: 1 - protein, 20 aa, Artificial Sequence
  polypeptide corresponding to positions 39-58 of human T-cell receptor Vbeta6 CDR2
LGQGPEFLTYFQNEAQLEKS

SEQ ID NO: 2 - protein, 21 aa, Artificial Sequence
  immunogenic polypeptide corresponding to positions 75-95 of human myelin basic protein
KSHGRTQDENPVVHFFKNIVT

SEQ ID NO: 3 - protein, 17 aa, Artificial Sequence
  Tiplimotide, immunogenic polypeptide corresponding to positions 83-99 of the human myelin basic protein
AKPVVHLFADIVTPRTP

SEQ ID NO: 4 - protein, 17 aa, Artificial Sequence
  immunogenic peptide derived from human myelin basic protein
DEDPVVHFFKDIVTPRT

SEQ ID NO: 5 - protein, 39 aa, Homo Sapiens
  PEPTIDE (0)...(0): Human ACTH
SYSMEHFRWGKPVGKKRRPVKVYPDGAEDELAEAFPLEF

(19) United States
(12) Patent Application Publication
(10) Pub. No.: US 2006/0189546 A1
(43) Pub. Date: Aug. 24, 2006

(54) Compounds for treating autoimmune and demyelinating diseases

(76) Inventors: Alfred M. Ajami, Brookline, MA (US); Michael A. Boss, Acton, MA (US); Jesse Paterson, Pointe Claire (CA)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/342,314

(22) Filed: Jan. 27, 2006

Related U.S. Application Data
(60) Provisional application No. 60/647,980, filed on Jan. 28, 2005.
(60) Provisional application No. 60/757,736, filed on Jan. 9, 2006.

(51) Int. Cl. A61K 31/7052; A61K 31/4745

(52) U.S. Cl. 514/23; 514/285

(57) ABSTRACT

A method of treating a patient suffering from an inflammatory and/or demyelinating disorders, comprising administering to said patient a therapeutically effective amount of a compound of formula (A) or a pharmaceutically acceptable salt thereof. 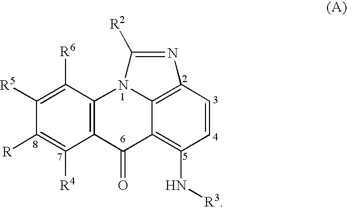 Definitions for the variables are provided therein.

### RELATED APPLICATIONS

This application claims the benefit of U.S. Provisional Application No. 60/647,980, filed Jan. 28, 2005 and U.S. Provisional Appliction No. 60/757,736, filed Jan. 9, 2006. The entire teachings of the above application(s) are incorporated herein by reference.

### BACKGROUND OF THE INVENTION

Autoimmune diseases, e.g., multiple sclerosis (MS), systemic lupus erythematosus (SLE), rheumatoid arthritis (RA), inflammatory bowel disease (IBD) and psoriasis represent assaults by the body's immune system which may be systemic in nature, or else directed at individual organs in the body. They appear to be diseases in which the immune system makes mistakes and, instead of mediating protective functions, becomes the aggressor.

Multiple sclerosis (MS) is a debilitating, inflammatory, neurological illness characterized by demyelination of the central nervous system. MS is the most common acquired neurologic disease of young adults in Western Europe and North America with a higher incidence in females. It accounts for more disability and financial loss, both in lost income and in medical care, than any other neurologic disease of this age group. There are approximately 250,000 cases of MS in the United States. Symptoms of the disease include fatigue, numbness, tremor, tingling, dysesthesias, visual disturbances, dizziness, cognitive impairment, urologic dysfunction, decreased mobility, and depression. Four types classify the clinical patterns of the disease: relapsing-remitting, secondary progressive, primary-progressive and progressive-relapsing (S. L. Hauser and D. E. Goodkin, Multiple Sclerosis and Other Demyelinating Diseases in Harrison's Principals of Internal Medicine 14^{th} Edition, vol. 2, Mc Graw-Hill, 1998, pp. 2409-2419).

MS affects the central nervous system and involves a demyelination process, i.e. the myelin sheaths are lost whereas the axons are preserved. In the later stages of disease there is damage to axons as well. Myelin provides the isolating material that enables rapid nerve impulse conduction. Evidently, in demyelination, this property is lost. The exact etiology of MS is unknown; although the pathogenic mechanisms responsible for MS are not understood, several lines of evidence indicate that demyelination has an immunopathologic basis with the demyelination characteristic of the disease a result of an autoimmune response perhaps triggered by an environmental insult, e.g. a viral infection. The pathologic lesions, the plaques, are characterized by infiltration of immunologically active cells such as macrophages and activated T cells. Specifically, it is hypothesized that MS is caused by a T-cell-mediated, autoimmune inflammatory reaction. The autoimmune basis is strongly supported by the fact that antibodies specific to myelin basic protein (MBP) have been found in the serum and cerebrospinal fluid of MS patients and these antibodies along with T-cells that are reactive to MBP and other myelin proteolipids increase with disease activity. Furthermore, at the cellular level it is speculated that T-cell proliferation and other cellular events, such as activation of B cells and macrophages and secretion of cytokines accompanied by a breakdown of the blood-brain barrier can cause destruction of myelin and oligodendrocytes. (R. A. Adams, M. V. Victor and A. H. Ropper eds, Principals of Neurology, Mc Graw-Hill, New York, 1997, pp. 903-921). Progressive MS (primary and secondary) may be based on a neurodegenerative process occurring with demyelination.

At the present time there is no cure for MS. Current therapies are aimed at alleviating the symptoms of the disease and arresting its progress, as much as possible. Depending upon the type, drug treatment usually entails the use of disease-modifying agents such as the interferons (interferon beta 1-a, beta 1-b, and alpha), glatiramer acetate or corticosteroids such as methylprednisolone and prednisone. Also, chemotherapeutic agents such as mitoxantrone, methotrexate, azathioprine, cladribine cyclophosphamide, cyclosporine and tysabri have been used. All of the above treatments have side effect liabilities, little or no effect on fatigue and depression, limited effects on relapse rates and on ability to prevent exacerbation of the disease. Treatment with interferons may also induce the production of neutralizing antibodies, which may ultimately decrease the efficacy of this therapy. Therefore, there still exists a strong need for new drugs, which can be used alone or in combination with other drugs to combat the progression and symptoms of MS. While considerable progress has been made in the of immunologic therapies, especially with the anti-integrin blocking antibody known as natalizumab introduced in 2005, no new small molecule treatments have yet emerged as generally accepted or widely available therapies, especially for chronic use in secondary progressive disease.

The progression of handicap is the main concern for patients with multiple sclerosis (MS) but most attempts to slow progression have been disappointing so far. Currently approved treatments with immunomodulators provide a modest or no benefit in secondary progressive MS (Lancet 1998, 54, 2352; Neurology 2000, 54, 2352; Neurology 2001, 56, 1496; Neurology 2002, 59, 679). Recent specific immunosuppressive therapies (monoclonal antibodies) were found able to eradicate exacerbations but without any significant benefit on progression (Neurology 1999, 53, 751).

General immunosuppression has been used in progressive MS for several decades but its efficacy is still debated. However, using treatment failure (TF) as clinical parameter (increase of 1 EDSS point confirmed at 3 months) and the “clinically significant benefit” as defined by Goodkin et al. (50% reduction in the TF rate in the treated group versus the placebo group), it appears that cyclosporine A (Ann Neurol 1990, 27, 591), methotrexate (Ann Neurol 1995, 37, 30) and azathioprine (Neurology 1989, 39, 1018) do not reach a clinically significant benefit. More potent immunosuppressive therapies provide a transient benefit which does not exceed 1 year. This was observed after short-term (2 months) total lymphoid irradiation (Lancet 1986, 8495, 1405) as well as with monthly administration of cyclophosphamide (CY) for 2 years (Arch Neurol 1987, 44, 823).

Accordingly, there is a need for a pharmaceutically acceptable immunomodulating therapy, that will arrest the neurodegeneration processes, including the ones triggered by inflammatory cell assault, with high clinical efficacy that provides long-lasting clinical benefit without significant side effects.

### SUMMARY OF THE INVENTION

The present invention is a method of treatment for inflammatory and demyelinating diseases, including multiple sclerosis. More specifically, the present invention is a method of treatment of certain inflammatory and demyelinating diseases by administration of derivatives of imidazoacridines.

In one embodiment, the present invention is a method of treating a patient suffering from an inflammatory disorder, comprising administering to said patient a therapeutically effective amount of a compound of formula (A) or a pharmaceutically acceptable salt thereof.

In another embodiment, the present invention is a method of treating a patient suffering from a demyelating condition, comprising administering to said patient a therapeutically effective amount of a compound of formula (A) or a pharmaceutically acceptable salt thereof.

In another embodiment, the present invention is a method of promoting remyelination of nerve cells in a patient in need thereof, comprising administering to the patient a therapeutically effective amount of a compound of formula (A) or a pharmaceutically acceptable salt thereof.

In another embodiment, the present invention is a composition comprising a therapeutically effective amount of a compound of formula (A) below, or pharmaceutically acceptable salt thereof, and an anti-inflammatory agent.

In another embodiment, the present invention is a method of reversing paralysis in a patient resulting from a demyelinating disease, comprising administering to the patient a compound in an amount sufficient to inhibit lymphocyte infiltration of immune cells in the spinal cord to promote remyelination of nerve cells in the spinal cord and thereby treating paralysis in said patient, wherein the compound is of formula formula (A) or a pharmaceutically acceptable salt thereof.

The imidazoacridines used in the present invention are described by formula (A): 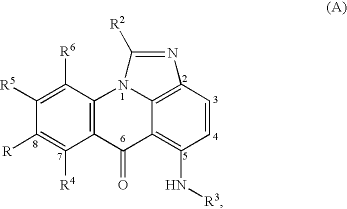
wherein:

R is —H, an optionally substituted alkyl, a hydroxyl, an alkoxy group, a halogen, a group represented by the following structural formula

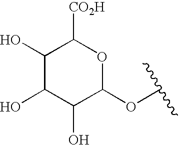

or, R and R⁵ taken together with their intervening carbon atoms form a 5, 6 or 7 member, optionally substituted, cycloalkyl or non-aromatic heterocycle;

or R and R⁴ taken together with their intervening carbon atoms form a 5, 6 or 7 member, optionally substituted, cycloalkyl or non-aromatic heterocycle; and

R² is —H, an optionally substituted C1-C10 alkyl or an optionally substituted aryl or heteroaryl;

R³ is —(CH₂)ₙ—NR^{a}R^{b}, wherein n=1-5, and R^{a} and R^{b}, each independently are hydrogen or an optionally substituted alkyl, or —NR^{a}R^{b} is an N-morpholinyl or N-pyrazinyl optionally substituted at one or more substitutable carbons with methyl, hydroxyl, or methoxy group, and wherein the N-pyrazinyl is optionally N′-substituted with C1-C4 alkyl or C1-C4 alkyl substituted with —NR^{c}R^{d}, wherein R^{c} and R^{d} are individually —H, methyl or ethyl; and

R⁴, R⁵ and R⁶, are each independently —H, —OH, a halogen or a C1-C6 alkoxy; or

R⁵ and R⁵ taken together with their intervening carbon atoms, form a 5, 6 or 7 member, optionally substitited cycloalkyl or non-aromatic heterocycle.

### BRIEF DESCRIPTION OF THE DRAWINGS

FIG. 1A is a bar plot illustrating inhibition of B-cells proliferation by Symadex™ following stimulation with lipopolysaccharide (LPS).

FIG. 1B is a bar plot illustrating inhibition of T-cells proliferation by Symadex™ following stimulation with concavanalin A (Con A).

FIG. 2A is a bar plot illustrating inhibition of IL-4 release by Symadex™ following stimulation with concavanalin A (Con A).

FIG. 2B is a bar plot illustrating inhibition of IL-10 release by Symadex™ following stimulation with concavanalin A (Con A).

FIG. 3 is a bar plot of the mean clinical score of the animals suffering from chronic stage Experimental Autoimmune Encephalomyelitis (EAE) at the indicated day post treatment with Symadex™ at 20 and 40 mg/kg versus vehicle control.

FIG. 4 is a time dependent chart showing mean clinical score (performance score) of the animals suffering from chronic stage Experimental Autoimmune Encephalomyelitis (EAE) at the indicated day post treatment with Symadex™ versus vehicle control.

FIG. 5 is a time dependent chart showing mean clinical score (performance score) of the animals suffering from chronic stage Experimental Autoimmune Encephalomyelitis (EAE) at the indicated day post treatment with Symadex™ after 4 weeks of dosing at 20 mg/kg versus vehicle control.

FIG. 6 is a time dependent chart showing mean clinical score (performance score) of the animals suffering from chronic stage Experimental Autoimmune Encephalomyelitis (EAE) at the indicated day post treatment with Symadex™ after 6 weeks of dosing at 20 mg/kg versus vehicle control.

FIG. 7 is a time dependent chart showing mean clinical score (performance score) of the animals suffering from chronic stage Experimental Autoimmune Encephalomyelitis (EAE) at the indicated day post treatment with Symadex™ after 8 weeks of dosing at 20 mg/kg versus vehicle control.

FIG. 8A is a bar chart showing the time course of T-cell counts of the animals suffering from chronic stage Experimental Autoimmune Encephalomyelitis (EAE) at the indicated day post treatment with Symadex™ after 4, 6 and 8 weeks of dosing at 20 mg/kg versus vehicle control.

FIG. 8B is a bar chart showing the time course of CD-4 cell counts of the animals suffering from chronic stage Experimental Autoimmune Encephalomyelitis (EAE) at the indicated day post treatment with Symadex™ after 4, 6 and 8 weeks of dosing at 20 mg/kg versus vehicle control.

FIG. 8C is a bar chart showing the time course of CD-8 cell counts of the animals suffering from chronic stage Experimental Autoimmune Encephalomyelitis (EAE) at the indicated day post treatment with Symadex™ after 4, 6 and 8 weeks of dosing at 20 mg/kg versus vehicle control.

FIG. 8D is a bar chart showing the time course of B-cell counts of the animals suffering from chronic stage Experimental Autoimmune Encephalomyelitis (EAE) at the indicated day post treatment with Symadex™ after 4, 6 and 8 weeks of dosing at 20 mg/kg versus vehicle control.

FIG. 9 is a time dependent chart showing mean clinical score (performance score) of the animals suffering from chronic stage Experimental Autoimmune Encephalomyelitis (EAE) at the indicated day post treatment with Symadex™ after 2 consecutive doses at 20 mg/kg given 72 hours apart versus vehicle control.

FIG. 10 is a time dependent chart showing mean clinical score of the animals suffering from acute stage Experimental Autoimmune Encephalomyelitis (EAE) at the indicated day post treatment with Symadex™ after 15 consecutive daily dose at 6 mg/kg.

FIG. 11 is a time dependent chart showing mean weight gain record of the animals suffering from acute stage Experimental Autoimmune Encephalomyelitis (EAE) at the indicated day post treatment with Symadex™ after 15 consecutive daily dose at 6 mg/kg.

FIG. 12 is a bar chart showing mean pathology scores on necropsy of the animals suffering from acute stage Experimental Autoimmune Encephalomyelitis (EAE) at the indicated day post treatment with Symadex™ after 15 consecutive daily dose at 6 mg/kg.

FIG. 13 is a time dependent chart bar chart showing mean performance of the animals suffering from acute stage Collagen Monoclonal Antibody (mAB) Induced Arthritis at the indicated day post treatment with Symadex™ after 3 consecutive daily oral doses at 30 mg/kg.

FIG. 14 is a list of chemical structures of pharmaceutical agents that can be co-administered with the compounds disclosed in the present invention.

### DETAILED DESCRIPTION OF THE INVENTION

It has now been discovered that administration of certain derivatives of imidazoacridines can treat and or alleviate the symptoms of various inflammatory diseases and diseases involving demyelination.

Specifically, it has been discovered that various inflammatory diseases and diseases involving demyelination can be treated by administering to a patient suffering from such a disease a therapeutically effective amount of a compound of formula (A) or a pharmaceutically acceptable salt thereof: 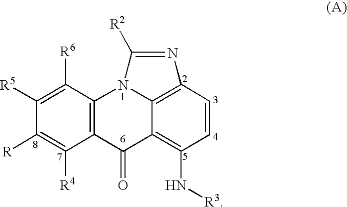
In formula (A), the substituents are each independently defined as follows.

R represents —H, an optionally substituted alkyl, a hydroxyl, an alkoxy group, a halogen or, R and R⁵, or alternatively R and R⁴, taken together with their intervening carbon atoms, form a 5, 6 or 7 member, optionally substitited cycloalkyl or non-aromatic heterocycle containing one or more oxygen, sulfur or optionally substituted nitrogen.

Preferably, R is —H; C1-C4 alkyl, optionally substituted with —OH, —SH, halogen, cyano, nitro, a C1-C3 alkyl, C1-C3 haloalkyl, C1-C3 alkoxy, C1-C3 haloalkoxy or C1-C3 alkyl sulfanyl, amine; C1-C2 alkylamine; or C1-C2 dialkylamine; or R and R⁵, or, alternatively, R and R⁴, taken together with their intervening carbon atoms form a 5-6 membered cycloalkyl or 5-6 membered non-aromatic heterocycle containing one or two oxygen atoms and optionally substituted with methyl or hydroxyl.

In one embodiment, R is represented by the following structure:

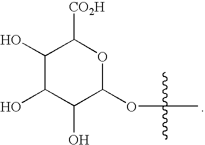

More preferably, R is —H, —OH, a, C1-C6 alkyl, a C1-C6 alkoxy group, —F, or, taken together with R⁴ or, alternatively, R⁵, forms a methylenedioxy group. More preferably, R is —H or a C1-C6 alkoxy group. Alternatively, R is an —OH or —OCH₃.

R² represents hydrogen, an optionally substituted C1-C10 alkyl or an optionally substituted aryl or heteroaryl. Preferably, R² is —H, C1-C8 alkyl, or phenyl, optionally substituted with one or more C1-C4 alkyl, C1-C4 alkoxy, C1-C4 haloalkoxy or cyano groups. More preferably, R² is —H or a C1-C4 alkyl.

R³ represents —(CH₂)ₙ—NR^{a}R^{b}, where n is an integer from 1 to 5, and R^{a} and R^{b}, which may be identical or different, represent hydrogen or an optionally substituted alkyl. Examples of substituents on such an alkyl include hydroxyl, a C1-C4 hydroxyalkyl, an amino, a N-alkyl-amino or a N,N-dialkylamino group.

Additionally, —NR^{a}R^{b} is an N-morpholinyl or N-pyrazinyl each optionally substituted at one or more substitutable carbons with methyl, hydroxyl, or methoxy, and wherein the N-pyrazinyl is optionally N′-substituted with C1-C4 alkyl or C1-C4 alkyl substituted with —NR^{c}R^{d}, wherein R^{c} and R^{d} are individually —H, methyl or ethyl.

Preferably, n is an integer from 2 to 3, and R^{a} and R^{b} are each independently —H, or a C1-C4 alkyl.

R⁴ and R⁶ are independently each —H, —OH, a halogen or a C1-C6 alkoxy. In some embodiments, R and R⁴, taken together with their intervening carbon atoms form a 5, 6 or 7 member, optionally substituted, cycloalkyl or non-aromatic heterocycle. When R and R⁴ are taken together with their intervening carbon atoms they preferably form a 5-6 membered cycloalkyl or 5-6 membered non-aromatic heterocycle containing one or two oxygen atoms and optionally substituted with methyl or hydroxyl; more preferably, R⁴ is —H, —OH, a C1-C3 alkoxy or taken together with R, forms a methylenedioxy group; and R⁶ is —H, —OH, or a C1-C3 alkoxy.

More preferably, R⁴ and R⁶, are independently each —H, —OH, or —OCH₃.

R⁵ is —H, —OH, a halogen, a C1-C6 alkoxy. In some embodiments, R and R⁵, taken together with their intervening carbon atoms form a 5, 6 or 7 member, optionally substituted, cycloalkyl or non-aromatic heterocycle. When R⁵ and R, or, alternatively, R⁵ and R⁶ are taken together with their intervening carbon atoms, they preferably form a 5-6 membered cycloalkyl or 5-6 membered non-aromatic heterocycle containing one or two oxygen atoms and optionally substituted with methyl or hydroxyl; more preferably, R⁵ is —H, —OH, a C1-C3 alkoxy or taken together with R, or, alternatively, R⁶, forms a methylenedioxy group.

In some embodiments, the substituents in formula (A) are defined as follows:

R is —H, C1-C4 alkyl, optionally substituted with —OH, —SH, halogen, cyano, nitro, a C1-C3 alkyl, C1-C3 haloalkyl, C1-C3 alkoxy, C1-C3 haloalkoxy or C1-C3 alkyl sulfanyl, amine, C1-C2 alkylamine or C1-C2 dialkylamine; or R and R⁵, taken together with their intervening carbon atoms form a 5-6 membered cycloalkyl or 5-6 membered non-aromatic heterocycle containing one or two oxygen atoms and optionally substituted with methyl or hydroxyl;

R² is —H, C1-C8 alkyl, or phenyl, optionally substituted with one or more C1-C4 alkyl, C1-C4 alkoxy, C1-C4 haloalkoxy or cyano groups;

R³ is —(CH₂)ₙ—NR^{a}R^{b}, n is an integer from 2 to 3, and R^{a} and R^{b} are each independently a hydrogen or an optionally substituted alkyl, or —NR^{a}R^{b} is an N-morpholinyl or N-pyrazinyl optionally substituted at one or more substitutable carbons with methyl, hydroxyl, or methoxy group, and wherein the N-pyrazinyl is optionally N′-substituted with C1-C4 alkyl or C1-C4 alkyl substituted with —NR^{c}R^{d}, wherein R^{c} and R^{d} are individually —H, methyl or ethyl;

R⁴, R⁵, and R⁶, are each independently —H, —OH, or C1-C3 alkoxy or, R⁴, or, alternatively, R⁵, taken together with R, form a methylenedioxy group.

Preferably, a compound of formula (A) is represented by formula (I):

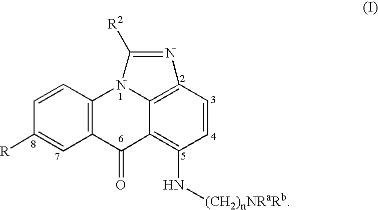

In formula (I), variables R, R², n, R^{a} and R^{b} can take values or preferred values defined above for formula (A). Preferred values for the variables in formual (I) are provided in the following paragraphs:

R represents a hydroxy or an alkoxy group, e.g., a C1-C6 alkoxy group. Alternatively, R is an —OH or —OCH₃;

R^{a} and R^{b}, which may be identical or different, can be hydrogen or an optionally substituted alkyl. Preferably, R^{a} and R^{b} are C1-C3 alkyls. More preferably, R^{a} and R^{b} are each independently ethyl. Alternatively, R^{a} and R^{b} are each methyl. In other embodiments, R^{a} and R^{b}, is each independently hydrogen or an optionally substituted alkyl.

when R^{a} or R^{b} are substituted alkyls, suitable substituents on the alkyls can include a hydroxyl, a C1-C4 hydroxyalkyl, an amino, a N-alkyl-amino or a N,N-dialkylamino groups, preferably containing 1-4 carbon atoms. Examples of such substituents are hydroxyethyl, aminoethyl, N-alkylaminoethyl and N,N-dialkylaminoethyl.

In other embodiments of formula (I), —NR^{a}R^{b} is an N-morpholinyl or N-pyrazinyl optionally substituted at one or more substitutable carbons with methyl, hydroxyl, or methoxy group, and wherein the N-pyrazinyl is optionally N′-substituted with C1-C4 alkyl or C1-C4 alkyl substituted with —NR^{c}R^{d}, wherein R^{c} and R^{d} are individually —H, methyl or ethyl.

Preferably, in formula (I) n is 2 or 3.

In formula (I), R² is a hydrogen or a C1-C6 alkyl. Preferably, R² is a hydrogen or a C1-C4 alkyl. More preferably, R² is a —H.

In some preferred embodiments of a compound of formula (I), R is —OH or —OCH₃, R^{a} and R^{b} are identical and represent C1-C6 alkyl groups, preferably, methyl or ethyl; n is 2 or 3; R² represents hydrogen or a straight chain C1-C4 alkyl. Preferably, R² is an —H.

Examples of compounds of formula (I) include compounds (IIA) through (IIH):

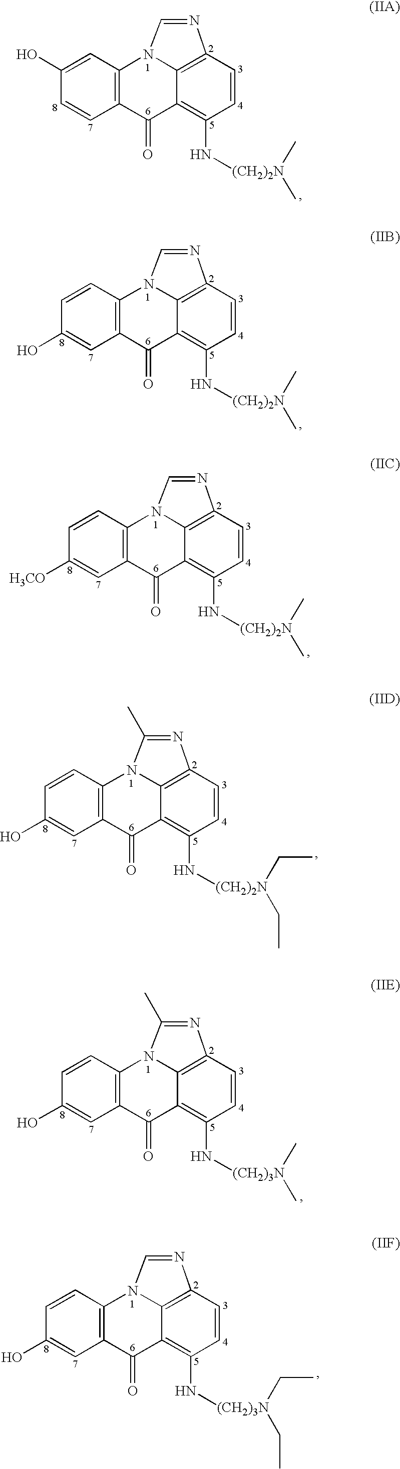

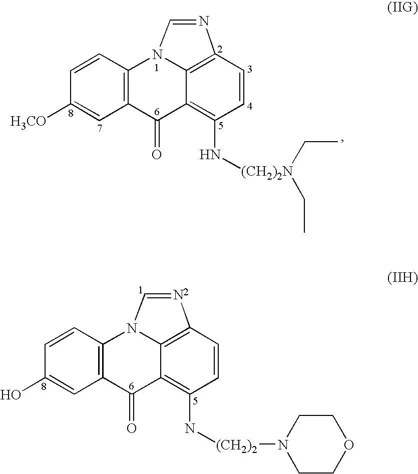

In a most preferred embodiment, the compound of formula (I) is 5-[[(diethylamino)ethyl]amino]-8-hydroxyimidazo[4,5,1-de]-acridine-6-one, whose structure is shown in formula (III):

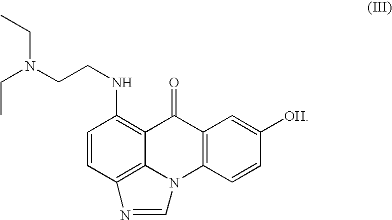

In another embodiment, a compound of formula (A) is represented by structural formula (IV):

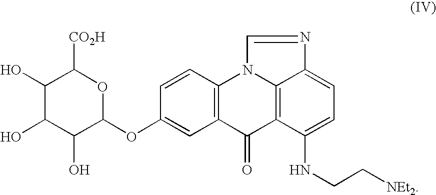

The term “alkyl”, as used herein, unless otherwise indicated, includes straight or branched saturated monovalent hydrocarbon radicals, typically C1-C10, preferably C1-C6. Examples of alkyl groups include, but are not limited to, methyl, ethyl, propyl, isopropyl, and t-butyl. Suitable substituents for a substituted alkyl include —OH, —SH, halogen, cyano, nitro, a C1-C3 alkyl, C1-C3 haloalkyl, C1-C3 alkoxy, C1-C3 haloalkoxy or C1-C3 alkyl sulfanyl.

The term “cycloalkyl”, as used herein, is a non-aromatic saturated carbocyclic moieties. Examples of cycloalkyl include, but are not limited to, cyclopropyl, cyclobutyl, cyclopentyl, cyclohexyl, and cycloheptyl. Suitable substituents for a cycloalkyl are defined above for an alkyl.

The term “haloalkyl”, as used herein, includes an alkyl substituted with one or more F, Cl, Br, or I, wherein alkyl is defined above.

The terms “alkoxy”, as used herein, means an “alkyl-O—” group, wherein alkyl, is defined above.

The term “haloalkoxy”, as used herein, means “haloalkyl-O—”, wherein haloalkyl is defined above.

As used herein, an amino group may be a primary (—NH₂), secondary (—NHRₓ), or tertiary (—NRₓR_{y}), wherein Rₓ and R_{y} may be any of the optionally substituted alkyls alkyls described above.

The term “aryl”, as used herein, refers to a carbocyclic aromatic group. Examples of aryl groups include, but are not limited to phenyl and naphthyl.

The term “heteroaryl”, as used herein, refers to aromatic groups containing one or more heteroatoms (O, S, or N). A heteroaryl group can be monocyclic or polycyclic, e.g. a monocyclic heteroaryl ring fused to one or more carbocyclic aromatic groups or other monocyclic heteroaryl groups. The heteroaryl groups of this invention can also include ring systems substituted with one or more oxo moieties. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

The term “non-aromatic heterocycle” refers to non-aromatic carbocyclic ring systems typically having four to eight members, preferably five to six, in which one or more ring carbons, preferably one to four, are each replaced by a heteroatom such as N, O, or S. Examples of non-aromatic heterocyclic rings include 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrorolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, and 1-pthalimidinyl.

The heteroaryl or non-aromatic heterocyclic groups may be C-attached or N-attached (where such is possible). For instance, a group derived from pyrrole may be pyrrol-1-yl (N-attached) or pyrrol-3-yl (C-attached).

Suitable substituents an aryl, a heteroaryl, or a non-aromatic heterocyclic group are those that do not substantially interfere with the pharmaceutical activity of the disclosed compound. One or more substituents can be present, which can be identical or different. Examples of suitable substituents for a substitutable carbon atom in a non-aromatic heterocyclic group include —OH, halogen (—F, —Cl, —Br, and —I), —R′, —OR′, —CH₂R′, —CH₂OR′, —CH₂CH₂OR′, —CH₂OC(O)R′, —O—COR′, —COR′, —SR′, —SCH₂R′, —CH₂SR′, —SOR′, —SO₂R′, —CN, —NO₂, —COOH, —SO₃H, —NH₂, —NHR′, —N(R′)₂, —COOR′, —CH₂COOR′, —CH₂CH₂COOR′, —CHO, —CONH₂, —CONHR′, —CON(R′)₂, —NHCOR′, —NR′COR′, —NHCONH₂, —NHCONR′H, —NHCON(R′)₂, —NR′CONH₂, —NR′CONR′H, —NR′CON(R′)₂, —C(═NH)—NH₂, —C(═NH)—NHR′, —C(═NH)—N(R′)₂, —C(═NR′)—NH₂, —C(═NR′)—NHR′, —C(═NR′)—N(R′)₂, —NH—C(═NH)—NH₂, —NH—C(═NH)—NHR′, —NH—C(═NH)—N(R′)₂, —NH—C(═NR′)—NH₂, —NH—C(═NR′)—NHR′, —NH—C(═NR′)—N(R′)₂, —NR′H—C(═NH)—NH₂, —NR′—C(═NH)—NHR′, —NR′—C(═NH)—N(R′)₂, —NR′—C(═NR′)—NH₂, —NR′—C(═NR′)—NHR′, —NR′—C(═NR′)—N(R′)₂, —SO₂NH₂, —SO₂NHR′, —SO₂NR′₂, —SH, —SOₖR′ (k is 0, 1 or 2) and —NH—C(═NH)—NH₂. Each R′ is independently an alkyl group.

Suitable substituents on the nitrogen of a non-aromatic heterocyclic group or a heteroaryl group include —R″, —N(R″)₂, —C(O)R″, —CO₂ R″, —C(O)C(O)R″, —C(O)CH₂C(O)R″, —SO₂R″, —SO₂N(R″)₂, —C(═S)N(R″)₂, —C(═NH)—N(R″)₂, and —NR″SO₂R″. R″ is hydrogen, an alkyl or alkoxy group.

Compounds (IIA) through (IIH) and (III) can be synthesized according to a variety of synthetic schemes disclosed in U.S. Pat. Nos. 5,231,100 and 6,229,015, incorporated herein by reference in their entirety. One example of such a scheme is shown below:

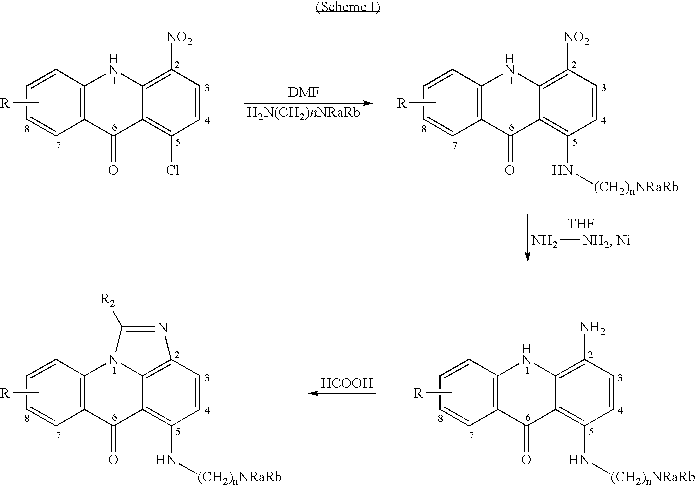

Compound (III) is known under the trade name of Symadex™. It has now been discovered, that Symadex™ inhibits proliferation of B-cells following stimulation with LPS and T-cells following stimulation with Con A as well as that Symadex™ inhibit release of cytokines such as IL-4 and IL-10 (Example 1). It has further been discovered in microarray experiments, that Symadex™ treatment results in altered expression of several genes involved in key regulatory pathways affecting the inflammatory and proliferative states, particularly the ability of invasive cells to assemble and aggregate, downregulation of cell proliferation and cell-cell signaling (Example 3). These molecular pharmacology studies show that Symadex™ exerts a downregulatory effect on genes implicated in mechanisms of cell aggregation and proliferation and on processes associated with invasive cellular growth, which are the hallmark of the inflammatory etiology associated with the autoimmune diseases. Taken together, these results indicate that Symadex™ can be used for treating the disorders that have inflammatory component, including autoimmune diseases.

It was further discovered that Symadex™ demonstrates activity in the female Hartley guinea pig Experimental Autoimmune Encephalomyelitis (EAE) model, a classic animal model for chronic-progressive MS (Example 2). Taken together with the results of Example 3, this result indicates that Symadex™ can be used for treating the disorders that have demyelinating as well as inflammatory components.

Accordingly, in one embodiment, the present invention is a method of treating a patient suffering from an inflammatory condition. The condition can be systemic lupus, inflammatory bowl disease, psoriasis, Crohn's disease, rheumatoid arthritis, sarcoid, Alzheimer's disease, a chronic inflammatory demyelinating neuropathy, insulin dependent diabetes mellitus, atherosclerosis, asthma, spinal cord injury or stroke.

Examples of chronic inflammatory demyelinating neuropathies include: chronic Immune Demyelinating Polyneuropathy (CIDP); multifocal CIDP; multifocal motor neuropathy (MMN); anti-MAG Syndrome (Neuropathy with IgM binding to Myelin-Associated Glycoprotein); GALOP Syndrome (Gait disorder Autoantibody Late-age Onset Polyneuropathy); anti-sulfatide antibody syndrome; anti-GM2 gangliosides antibody syndrome; POEMS syndrome (Polyneuropathy Organomegaly Endocrinopathy or Edema M-protein Skin changes); perineuritis; and IgM anti-GD1b ganglioside antibody syndrome.

The method comprises administering to a patient a therapeutically effective amount of a compounds of formula (A) or a pharmaceutically acceptable salt thereof. For example, compounds of formulae (IIA) through (IIH) can be used. Preferably, compound of formula (III) is used. Alternatively, the compound of formula (IV) is sued.

In another embodiment, the present invention is a method of treatment of a patient suffering from a demyelinating condition. As used herein, a “demyelinating condition” is a condition that destroys, breaks the integrity of or damages a myelin sheath. As used herein, the term “myelin sheath” refers to an insulating layer surrounding vertebrate peripheral neurons, that increases the speed of conduction and formed by Schwann cells in the peripheral or by oligodendrocytes in the central nervous system. Such condition can be multiple sclerosis, a congenital metabolic disorder, a neuropathy with abnormal myelination, drug-induced demyelination, radiation induced demyelination, a hereditary demyelination condition, a prion-induced demyelination, encephalitis-induced demyelination, a spinal cord injury, Alzheimer's disease as well as chronic inflammatory demyelinating neuropathies, examples of which are given above. In one embodiment, the condition is multiple sclerosis. The method comprises administering to a patient a therapeutically effective amount of a compound of formula (A) or a pharmaceutically acceptable salt thereof. For example, compounds of formulae (IIA) through (IIH) can be used. Preferably, compound of formula (III) is used. Alternatively, the compound of formula (IV) is used.

The term “patient” means a warm blooded animal, such as for example rat, mice, dogs, cats, guinea pigs, and primates such as humans. The terms “treat” or “treating” include any treatment, including, but not limited to, alleviating symptoms, eliminating the causation of the symptoms either on a temporary or permanent basis, or preventing or slowing the appearance of symptoms and progression of the named disorder or condition. The term “therapeutically effective amount” means an amount of the compound, which is effective in treating the named disorder or condition. In certain embodiments, therapeutically effective amount means an amount sufficient to effect remyelination of nerve cells in a patient.

In another embodiment, the present invention is a method of promoting remyelination of nerve cells in a patient, comprising administering to the patient in need thereof a therapeutically effective amount of a compound of formula I, formulae (IIA)-(IIH), formula (III) or formula (IV) or a pharmaceutically acceptable salt thereof. The patient can be suffering from any of the demyelinating conditions listed above.

In another embodiment, the present invention is a method of preventing demyelination and promoting remyelination in a patient in need thereof, comprising administering a combination of a therapeutically effective amount of a compound of formula I, formulae (IIA)-(IIH), formula (III) or formula (IV), or pharmaceutically acceptable salt thereof, and an anti-inflammatory agent as described below.

In another embodiment, the present invention is a method of reversing paralysis in a subject in need thereof with a demyelinating disease, comprising administering to the subject a compound in an amount sufficient to inhibit lymphocyte infiltration of immune cells in the spinal cord to promote remyelination of nerve cells in the spinal cord and thereby treating paralysis in said subject, wherein the compound is of formula formula I, formulae (IIA)-(IIH), formula (III) or formula (IV) or a pharmaceutically acceptable salt thereof.

The dosage range at which the disclosed compounds of formula (A), including compounds of formulae (IIA)-(IIH), (III) and (IV), exhibit their ability to act therapeutically can vary depending upon the severity of the condition, the patient, the formulation, other underlying disease states that the patient is suffering from, and other medications that may be concurrently administered to the patient. Generally, the inventive compounds of the invention will exhibit their therapeutic activities at dosages of between about 0.001 mg/kg of patient body weight/day to about 100 mg/kg of patient body weight/day. For example, the dosage can be 0.1-100 mg/kg, 1-100 mg/kg, 10-100 mg/kg, 1-50 mg, kg, 10-50 mg/kg or 10-30 mg/kg per day, per every other day or per week.

In other embodiments, compounds can be administered by any of the routes described below, preferably intravenously, in an amount from 1 mg per kilogram body weight to 20 mg per kg body weight. Compounds can be administered daily, once every 72 hours or weekly.

In one embodiment in which compounds are used to treat rheumatoid arthritis, compounds can be administered orally in an amount of 1-50 mg/kg, 10-40 mg/kg, 20-30 mg/kg or 30 mg per kilogram of body weight per day, per every other day or per week.

In one embodiment, the compounds of the invention are administered chronically to the patient in need thereof. For example, the chronic administration of the compound is daily, weekly, biweekly, or monthly over a period of at least one year, at least two years, at least three or more years.

In one embodiment, the compounds of formula (A), including compounds of formulae (IIA)-(IIH), (III) and (IV) are administered intravenously in the amount of 1.5-30 mg/kg once at intervals of 1-3 months. In another embodiment, the compounds are administered orally in the amount of 5-100 mg/kg on same schedule as above. Alternatively, the compounds of formula (A) are administered several times over a period of up to 3 months and up to a cumulative dose of between 1.5 and 30 mg/kg. In another embodiment, the cumulative dose is from 5 to 100 mg/kg.

In another embodiment, the compounds of formula (A) are administered intravenously in the amount of 2.5-10 mg/kg weekly for 8-24 weeks, repeating as needed after 6-18 weeks off drug. Alternatively, the compounds of formula (A) are administered several times over a period of from 14 weeks to 42 weeks to achieve a cumulative dose from 20 mg/kg to 240 mg/kg. Administration can be repeated over one or more periods of 14-42 weeks.

In another embodiment, the compounds of formula (A) are administered intravenously in the amount of 2.5-10 mg/kg twice, 72 hrs apart for 1 to 2 weeks, repeating monthly. Alternatively, the compounds of formula (A) are administered several times over a period of up to two weeks, up to a cumulative dose of from 11 mg/kg to 47 mg/kg. Administration can be repeated monthly.

In another embodiment, the compounds of formula (A) are administered orally in the amount of 1-3 mg/kg daily for 10-15 days, repeating every 30-45 days. Alternatively, the compounds of formula (A) are administered several times over a period of up to 40-60 days, up to a cumulative dose of from 10 mg/kg to 45 mg/kg. Administration can be repeated over one or more periods of up to 40-60 days.

In another embodiment, the compounds of the invention are administered orally in the amount of 2-6 mg/kg daily for 3 days per week, repeating every 15-30 days. Alternatively, the compounds of formula (A) are administered several times over a period of up to 30 days up to a cumulative dose of 6-18 mg/kg. Administration can be repeated over one or more periods of up to 30 days.

Preferably, the administration of the compounds or the combinations of the compounds described herein results in an effective blood level of the compound in the patient of more than or equal to 10 ng/ml. For example, compounds can be administered intravenously in an amount of 20 μg to about 500 μg per kilogram body weight of the patient.

Preferred human doses for treating chronic (remitting-relapsing) multiple sclerosis (MS) are 0.1 mg/kg to 10 mg/kg, 1-10 mg/kg, 1-5 mg/kg, 2-7 mg/kg, 2-5 mg/kg. Schedule could be once a month, twice a month, three times a month or once or twice a week for 3 months, 6 month, 12 months or more.

Preferred human doses for treating acute MS, is 0.1 mg/kg to 10 mg/kg, 0.1-5 mg/kg, 0.1-2 mg/kg, 0.5-2 mg/kg or 0.5-1 mg/kg three times a day, twice a day, or daily, on a weekly, biweekly or monthly basis.

Preferred human doses for treating rheumatoid arthritis 0.1 mg/kg to 10 mg/kg, 1-10 mg/kg, 1-5 mg/kg, 2-7 mg/kg, 2-5 mg/kg three times a day, twice a day, or daily, on a weekly, biweekly or monthly basis.

In treating a patient afflicted with a condition described above, all of the disclosed compounds can be administered in any form or mode which makes the compound bioavailable in therapeutically effective amounts. For example, compounds of formula (A) can be administered in a form of a pharmaceutically acceptable salt. The term “pharmaceutically acceptable salts” means either an acid addition salt or a basic addition salt, whichever is possible to make with the compounds of the present invention. “Pharmaceutically acceptable acid addition salt” is any non-toxic organic or inorganic acid addition salt of the base compounds represented by formula (A). Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tri-carboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicyclic, 2-phenoxybenzoic, p-toluenesulfonic acid and sulfonic acids such as methanesulfonic acid and 2-hydroxyethanesulfonic acid. Either the mono- or di-acid salts can be formed, and such salts can exist in either a hydrated or substantially anhydrous form. In general, the acid addition salts of these compounds are more soluble in water and various hydrophilic organic solvents and which in comparison to their free base forms, generally demonstrate higher melting points. “Pharmaceutically acceptable basic addition salts” means non-toxic organic or inorganic basic addition salts of the compounds of formula (A), including formulae (IIA)-(IIH), (III) and (IV). Examples are alkali metal or alkaline-earth metal hydroxides such as sodium, potassium, calcium, magnesium or barium hydroxides; ammonia, and aliphatic, alicyclic, or aromatic organic amines such as methylamine, trimethylamine and picoline. The selection of the appropriate salt may be important so that the ester is not hydrolyzed. The selection criteria for the appropriate salt will be known to one skilled in the art.

Compounds of the present invention can be administered by a number of routes including orally, sublingually, buccally, subcutaneously, intramuscularly, intravenously, transdermally, intranasally, rectally, topically, and the like. One skilled in the art of preparing formulations can determine the proper form and mode of administration depending upon the particular characteristics of the compound selected for the condition or disease to be treated, the stage of the disease, the condition of the patient and other relevant circumstances. For example, see Remington's Pharmaceutical Sciences, 18^{th} Edition, Mack Publishing Co. (1990), incorporated herein by reference.

The compound of formula (A) of this invention may also be administered topically, and when done so the carrier may suitably comprise a solution, ointment or gel base. The base, for example, may comprise one or more of petrolatum, lanolin, polyethylene glycols, bee wax, mineral oil, diluents such as water and alcohol, and emulsifiers and stabilizers.

The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampules, disposable syringes or multiple dose vials.

The compounds used in the present invention can be administered alone or in combination with one or more other pharmaceutically active agents that are effective against the inflammatory condition and/or the demyelating disorder being treated.

As used herein, the term “combination” with reference to pharmaceutically active agents and the term “co-administering” and “co-administration” refer to administering more than one pharmaceutically active agent to a patient during one treatment cycle and not necessarily simultaneous or in a mixture.

In one embodiment, the compounds of the present invention are administered in combination with an anti-inflammatory agent. The anti-inflammatory agent can be adrenocorticotropic hormone, a corticosteroid, an interferon, glatiramer acetate, or a non-steroidal anti-inflammatory drug (NSAID).

Examples of suitable anti-inflammatory agents include corticosteroid such as prednisone, methylprednisolone, dexamethasone cortisol, cortisone, fludrocortisone, prednisolone, 6α-methylprednisolone, triamcinolone, or betamethasone.

Other examples of suitable anti-inflammatory agents include NSAIDs such as aminoarylcarboxylic acid derivatives (e.g., Enfenamic Acid, Etofenamate, Flufenamic Acid, Isonixin, Meclofenamic Acid, Niflumic Acid, Talniflumate, Terofenamate and Tolfenamic Acid), arylacetic acid derivatives (e.g., Acematicin, Alclofenac, Amfenac, Bufexamac, Caprofen, Cinmetacin, Clopirac, Diclofenac, Diclofenac Sodium, Etodolac, Felbinac, Fenclofenac, Fenclorac, Fenclozic Acid, Fenoprofen, Fentiazac, Flubiprofen, Glucametacin, Ibufenac, Ibuprofen, Indomethacin, Isofezolac, Isoxepac, Ketoprofen, Lonazolac, Metiazinic Acid, Naproxen, Oxametacine, Proglumrtacin, Sulindac, Tenidap, Tiramide, Tolectin, Tolmetin, Zomax and Zomepirac), arylbutyric acid ferivatives (e.g., Bumadizon, Butibufen, Fenbufen and Xenbucin) arylcarboxylic acids (e.g., Clidanac, Ketorolac and Tinoridine), arylproprionic acid derivatives (e.g., Alminoprofen, Benoxaprofen, Bucloxic Acid, Carprofen, Fenoprofen, Flunoxaprofen, Flurbiprofen, Ibuprofen, Ibuproxam, Indoprofen, Ketoprofen, Loxoprofen, Miroprofen, Naproxen, Oxaprozin, Piketoprofen, Piroprofen, Pranoprofen, Protinizinic Acid, Suprofen and Tiaprofenic Acid), pyrazoles (e.g., Difenamizole and Epirizole), pyrazolones (e.g., Apazone, Benzpiperylon, Feprazone, Mofebutazone, Morazone, Oxyphenbutazone, Phenylbutazone, Pipebuzone, Propyphenazone, Ramifenazone, Suxibuzone and Thiazolinobutazone), salicyclic acid derivatives (e.g., Acetaminosalol, 5-Aminosalicylic Acid, Aspirin, Benorylate, Biphenyl Aspirin, Bromosaligenin, Calcium Acetylsalicylate, Diflunisal, Etersalate, Fendosal, Flufenisal, Gentisic Acid, Glycol Salicylate, Imidazole Salicylate, Lysine Acetylsalicylate, Mesalamine, Morpholine Salicylate, 1-Naphthyl Sallicylate, Olsalazine, Parsalmide, Phenyl Acetylsalicylate, Phenyl Salicylate, 2-Phosphonoxybenzoic Acid, Salacetamide, Salicylamide O-Acetic Acid, Salicylic Acid, Salicyloyl Salicylic Acid, Salicylsulfuric Acid, Salsalate and Sulfasalazine), thiazinecarboxamides (e.g., Droxicam, Isoxicam, Piroxicam and Tenoxicam), ε-Acetamidocaproic Acid, S-Adenosylmethionine, 3-Amino-4-hydroxybutyric Acid, Amixetrine, Bendazac, Benzydamine, Bucolome, Difenpiramide, Ditazol, Emorfazone, Guaiazulene, Ketorolac, Meclofenamic Acid, Mefenamic Acid, Nabumetone, Nimesulide, Orgotein, Oxaceprol, Paranyline, Perisoxal, Pifoxime, Piroxicam, Proquazone, Tenidap and a COX-2 inhibitor (e.g., Rofecoxib, Valdecoxib and Celecoxib).

Further examples of anti-inflammatory agents include aspirin, a sodium salicylate, choline magnesium trisalicylate, salsalate, diflunisal, sulfasalazine, olsalazine, a para-aminophenol derivatives, an indole, an indene acetic acid, a heteroaryl acetic acid, an anthranilic acid, an enolic acid, an alkanones, a diaryl-substituted furanone, a diaryl-substituted pyrazoles, an indole acetic acids, or a sulfonanilide.

In some embodiments, the compounds of the present invention can be administered in combination with immunotherapeutic agents such as interferons and anti-integrin blocking antibodies like natalizumab.

Examples of agents suitable for treating demyelinating disorders include Pirfenidone, Epalrestat, Nefazodone hydrochloride, Memantine hydrochloride, Mitoxantrone hydrochloride, Mitozantrone hydrochloride, Thalidomide, Roquinimex, Venlafaxine hydrochloride, Intaxel, Paclitaxel, recombinant human nerve growth factor; nerve growth factor, ibudilast, Cladribine, Beraprost sodium, Levacecarnine hydrochloride; Acetyl-L-carnitine hydrochloride; Levocarnitine acetyl hydrochloride, Droxidopa, interferon alfa, natural interferon alpha, human lymphoblastoid interferon, interferon beta-1b, interferon beta-Ser, Alemtuzumab, Mycophenolate mofetil, Zoledronic acid monohydrate, Adapalene, Eliprodil, Donepezil hydrochloride, Dexanabinol, Dexanabinone, Xaliproden hydrochloride, interferon alfa-n3, lipoic acid, thioctic acid, Teriflunomide, Atorvastatin, Pymadin, 4-Aminopyridine, Fampridine, Fidarestat, Priliximab, Pixantrone maleate, Dacliximab, Daclizumab, Glatiramer acetate, Rituximab, Fingolimod hydrochloride, interferon beta-1a, Natalizumab, Abatacept, Temsirolimus, Lenercept, Ruboxistaurin mesilate hydrate, Dextromethorphan/quinidine sulfate, Capsaicin, Dimethylfumarate or Dronabinol/cannabidiol.

In some embodiments, the compounds of the present invention can be administered in combination with one or more other pharmaceutically active agents that are effective against multiple sclerosis. Examples of such agents include the interferons (interferon beta 1-a, beta 1-b, and alpha), glatiramer acetate or corticosteroids such as methylprednisolone and prednisone as well as chemotherapeutic agents such as mitoxantrone, methotrexate, azathioprine, cladribine cyclophosphamide, cyclosporine and tysabri.

Further examples of pharmaceutically active agents that are effective against multiple sclerosis and are suitable to be administered in combination with compounds of the present invention include compounds of the following structural formulae:

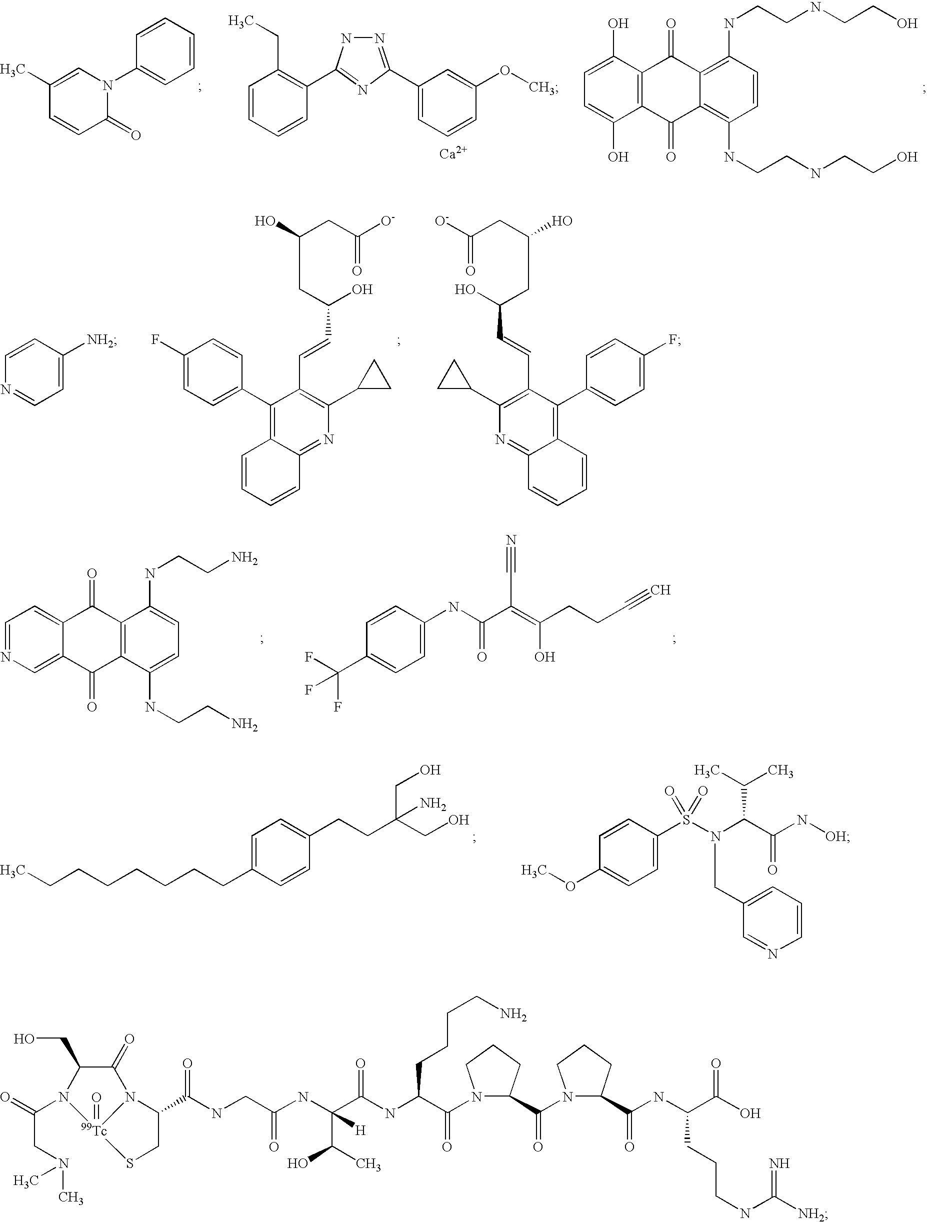

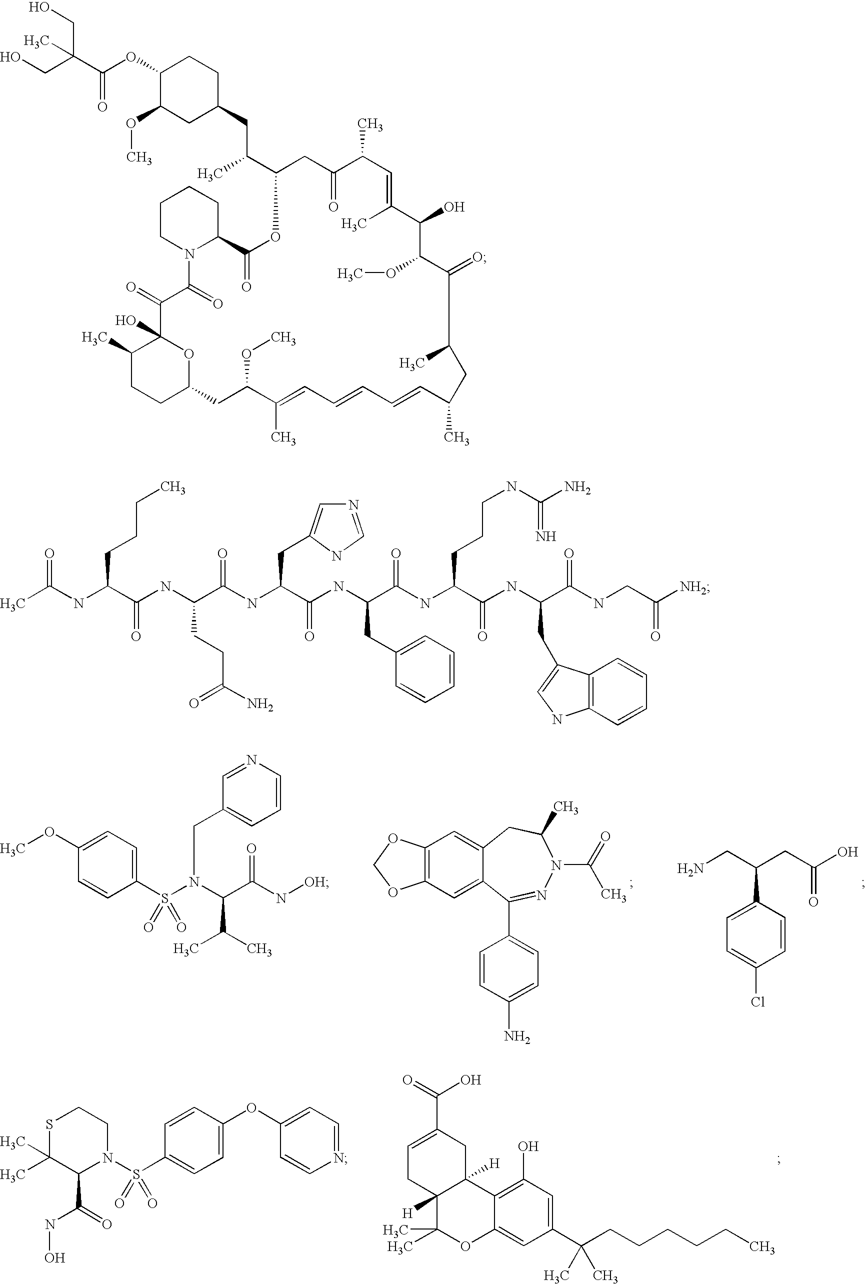

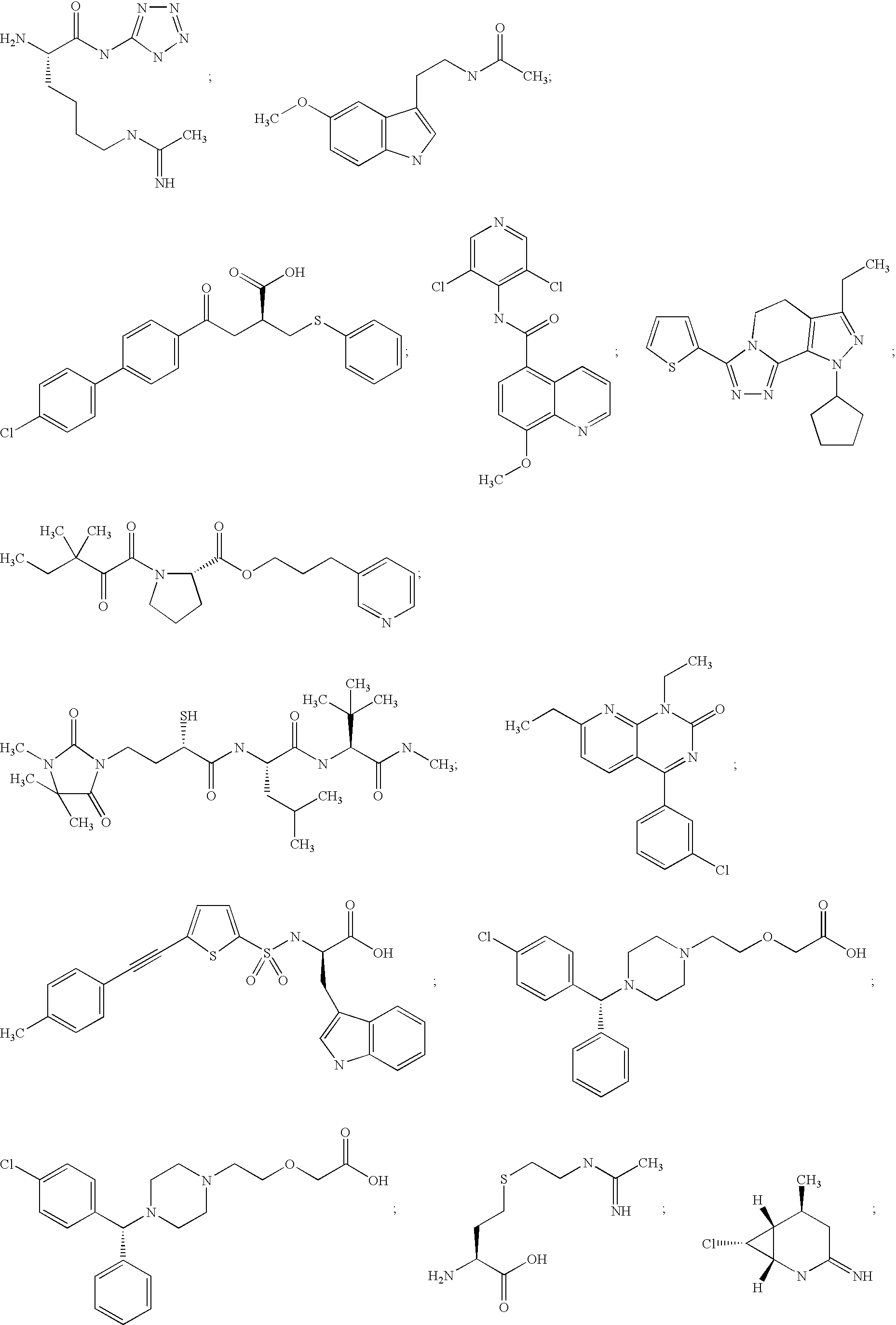

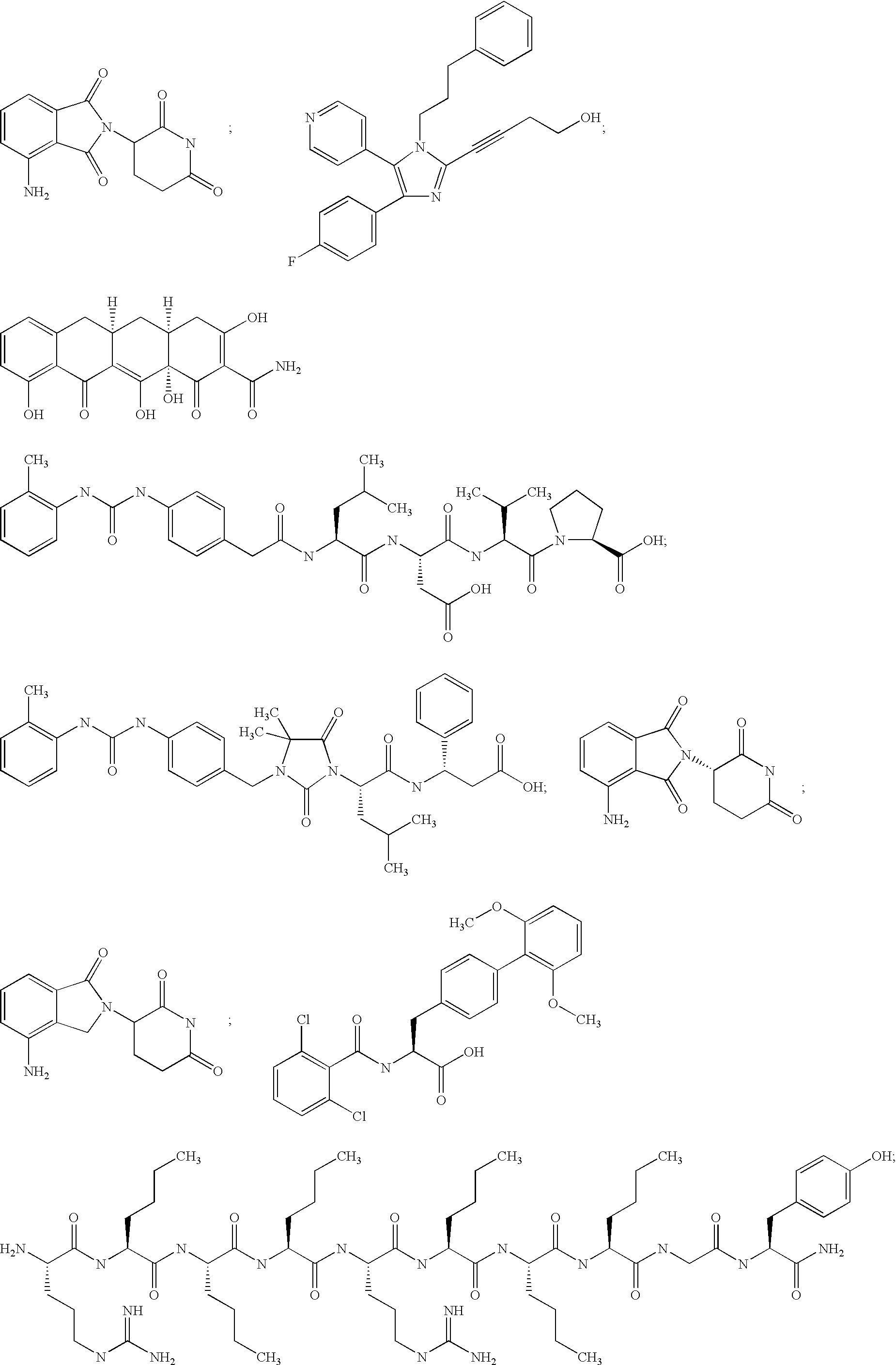

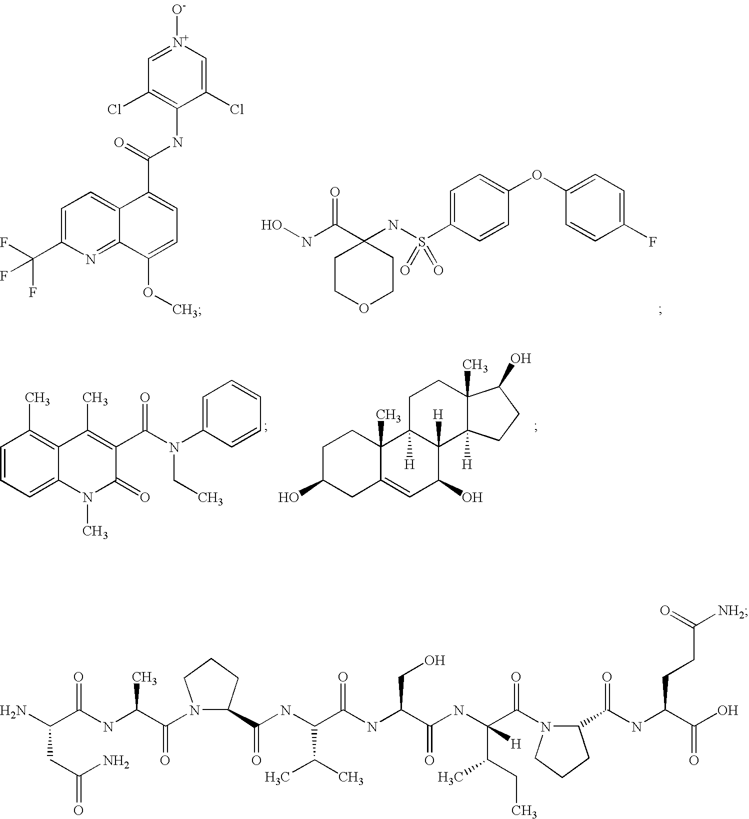

Further examples of pharmaceutical agents that can be co-administered with the compounds of formula (A) include:

T-cell receptor (TCR) Vβ6 CDR2 peptide vaccine consisting of TCR Vβ6, amino acid sequence 39-58, Leu-Gly-Gln-Gly-Pro-Glu-Phe-Leu-Thr-Tyr-Phe-Gln-Asn-Glu-Ala-Gln-Leu-Glu-Lys-Ser (SEQ ID NO:1);

Myelin basic protein immunogen peptide, aminoacid sequence 75-95, Lys-Ser-His-Gly-Arg-Thr-Gln-Asp-Glu-Asn-Pro-Val-Val-His-Phe-Phe-Lys-Asn-Ile-Val-Thr (SEQ ID NO:2);

Tiplimotide, myelin basic protein immunogen vaccine peptide, aminoacid sequence 83-99, D-Ala-lys-pro-val-val-his-leu-phe-ala-asp-ile-val-thr-pro-arg-thr-pro, (SEQ ID NO:3);

Myelin basic protein immunogen peptide, aminoacid sequence 82-98, Asp-glu-asp-pro-val-val-his-phe-phe-lys-asp-ile-val-thr-pro-arg-thr, (SEQ ID NO:4);

Adrenocorticotropic hormone (ACTH), Ser-Tyr-Ser-met-glu-his-phe-arg-try-gly-lys-pro-val-gly-lys-lys-arg-arg-pro-val-lys-val-tyr-pro-asp-gly-ala-glu-asp-glu-leu-ala-glu-ala-phe-pro-leu-glut-phe, (SEQ ID NO:5).

Further examples of pharmaceutically active agents that are effective against multiple sclerosis and are suitable to be administered in combination with compounds of the present invention include:

3-4 diaminopyridine; ABT-874; Actos® (pioglitazone); ALCAR (acetyl-L-carnitine); Alpha lipoic acid; AndroGel® (testosterone gel); combination of trimethoprim and vitamin C; combination of azithromycin and rifampin; minocycline; donezepil HCL; Avandia® (rosiglitazone maleate; combination of IFN beta-1a) and acetaminophen, ibuprofen or prednisone; combination of Avonex® (interferon beta-1a)+CellCept® (mycophenolate mofetil); combination of Avonex® (interferon beta-1a) and Copaxone® (glatiramer acetate); combination of Avonex® (interferon beta-1a) and doxycycline; combination of Avonex® (interferon beta-1a) and EMLA (lidocaine and prilocaine) anesthetic cream; Avonex® (interferon beta-1a) and estrogen and progesterone; combination of Avonex® (interferon beta-1a)+Fludara® (fludarabine phosphate); combination of Avonex® (interferon beta-1a) and methotrexate and leucovorin rescue; combination of Avonex® (interferon beta-1a) and methotrexate and methylprednisolone; combination of Avonex® (interferon beta-1a) and Novantrone® (mitoxantrone); combination of Avonex® (interferon beta-1a) and Prozac® (fluoxetine); combination of Avonex® (interferon beta-1a) and Topamax® (topiramate); combination of Avonex® (interferon beta-1a) and Zocor® (simvastatin); AVP-923 (dextromethorphan/quinidine); combination of Betaseron® (interferon beta-1b) and Imuran® (azathioprine); combination of Betaseron® (interferon beta-1b) and Copaxone® (glatiramer acetate); combination of BHT-3009-01 and Lipitor® (atorvastatin); Bone marrow/peripheral stem cell transplant; CellCept® (mycophenolate mofetil); combination of CellCept® (mycophenolate mofetil) and Avonex® (interferon beta-1a); Oral cladribine; CNTO 1275 (monoclonal antibody); combination of Copaxone® (glatiramer acetate) and Antibiotic therapy (minocycline); combination of Copaxone® (glatiramer acetate) and Novantrone® (mitoxantrone); combination of Copaxone® (glatiramer acetate) and prednisone; combination of Copaxone® (glatiramer acetate) and Proventil® (albuterol); Cyclophosphamide; Daclizumab; Deskar® (pirfenidone); Estriol; Fumaric acid esters; Gabitril® (tiagabine HCL); Ginkgo biloba; IDEC-131 (anti-CD40L or anti-CD 154); the combination of Immunoglobulin and methylprednisolone; Inosine; Interferon tau; Lamictal® (lamotrigine); Lexapro® (escitalopram); Lipitor® (atorvastatin); combination of Lipitor® (atorvastatin) and Rebif® (interferon beta-1a); combination of Lymphocytapheresis (removal of immune cells), Imuran® (azathioprine) and prednisone; MBP8298; Methylprednisolone; combination of Methylprednisolone and Avonex (interferon beta-1a); Modiodal (modafinil); NBI-5788 (altered peptide ligand); combination of Novantrone® (mitoxantrone for injection concentrate) and Avonex® (Interferon beta-1a) or Copaxone® (glatiramer acetate); Omega-3 Fatty Acid Supplementation; Pixantrone (BBR 2778); combination of Provigil® (modafinil) and Avonex® (interferon beta-1a); Rapamune® (sirolimus); RG2077; Rituxan® (rituximab); Rolipram (phosphodiesterase-4 inhibitor); SAIK-MS (laquinimod, ABR-215062); T cell vaccination; Teriflunomide; Tetrahydrocannabinol; Tetrahydrocannabinol (dronabinol); Thalamic stimulation; combination of Tysabri® (natalizumab) and Avonex® (interferon beta-1a); combination of Tysabri® (natalizumab) and Copaxone® (glatiramer acetate); and Viagra® (sildafenil citrate).

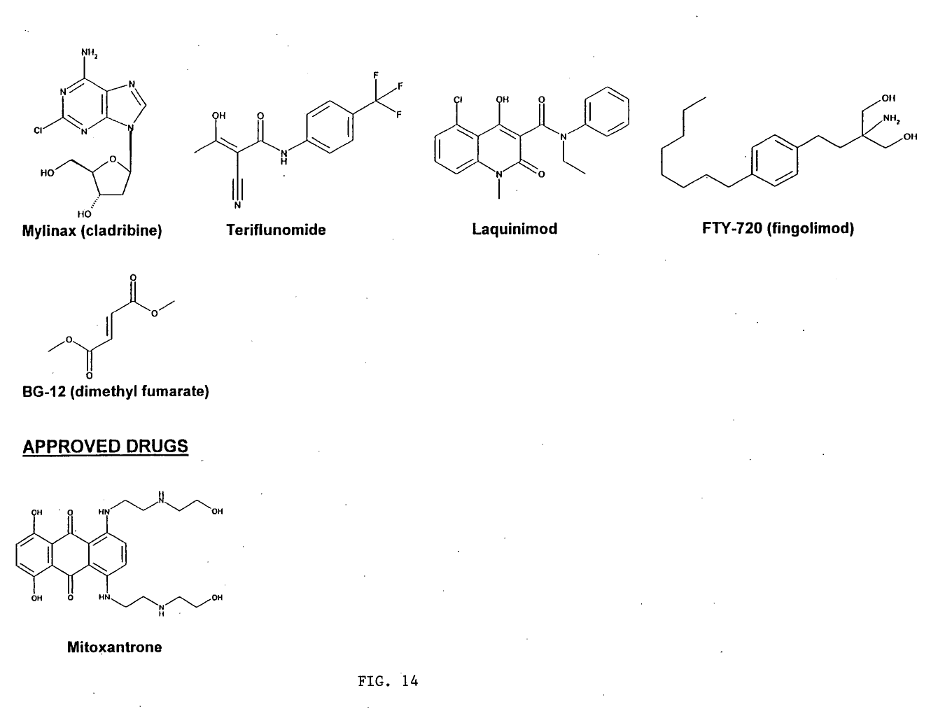

Further examples of pharmaceutically active agents that are effective against multiple sclerosis and are suitable to be administered in combination with compounds of the present invention include compounds listed in FIG. 14. Additionally, Copaxone (Glatiramer) can be orally co-administered with the compounds of the present invention.

In other embodiments, pharmaceutically active agents that are effective against multiple sclerosis and are suitable to be administered in combination with compounds of the present invention include compounds include: Mylinax, an oral formulation of cladrlbine used in leukaemia treatment, developed by Serono/Ivex; Teriflunomide, a metabolite of Arava, an oral immunosuppressant, developed by Sanofl-Aventis; FTY 720, an oral immunomodulator (Sphingosine-1-phosphate receptor agonist), developed by Novartis; MBP 8298, a synthetic myelin basis protein designed to reduce the emergence of antibodies directed against the myelin, developed by Bio MS Medical; an orphan drug 4-aminopyridline (4-AP), a potassium channel blocker, developed by Acorda; Gamunex, an intravenous immunoglobulin formulation, developed by Bayer; BG-12 fumarate, a second generation oral futnarate, developed by Biogen Idec/Fumapharm; Temsirolimus, a T-lymphocytes proliferation blocker, developed by Wyeth; E-2007, an AMPA receptor agonist, developed by Eisal; Campath, a humanized antibody directed against CD52, developed by Genzyme; Neuro Vax, a vaccine, developed by Immune Response; Zocor, a statin, developed by Merck; NBI 5788, a myelin-mimicking peptide ligand, developed by Neurocrine; Tauferon, Interferon tau, developed by Pepgen; Zenapax, a humanized anti-CD25 immunosuppressive antibody, developed by Protein Design; a combination of MS-IET and EMZ 701, a methyl donator, developed by Transition Therapeutics; Laquinlmod, an oral formulation of a derivative of linomide, developed by Active Biotech/Teva; deskar pirfenidone, a TNF-alpha inhibitor, developed by Mamac; ATL-1102, a second generation antisense inhibitor targeting VLA4, developed by Antisense Therapeutics.

In some embodiments, compounds of formula (A) can be administered in combination with antivascular agents, in particular agents inhibiting the growth factor receptors, Epidermal Growth Factor Receptor (EGFR), Vascular Epidermal Growth Factor Receptor (VEGFR), and Fibroblast Growth Factor Receptor (FGFR). Examples of such agents include, Iressa, Tarceva, Erbitux, Pelitinib, AEE-788, CP-547632, CP-547623, Tykerb (GW-2016), INCB-7839, ARRY-334543, BMS-599626, BIBW-2992, Falnidamol, AG1517, E-7080, KRN-951, GFKI-258, BAY-579352, CP-7055, CEP-5214, Sutent, Macugen, Nexavar, Neovastat, Vatalanib succinate, GW-78603413, Lucentis, Teavigo, AG-13958, AMG-706, Axitinib, ABT-869, Evizon, Aplidin, NM-3, PI-88, Coprexa, AZD-2171, XL-189, XL-880, XL-820, XL-647, ZK-CDK, VEGFTrap, OSI-930, Avastin, Revlimid, Endostar, Linomide, Xinlay, SU-668, BIBF-1120, BMS-5826624, BMS-540215.

In some embodiments, compounds of formula (A), including compounds of formulae (I)-(IV) can be administered in combination with agents that affect T-cell homing, extravastion and transmigration. Examples of such agents include, FTY-720PKI-166, PTK-787, SU-11248.

In some embodiments, compounds of formula (A), including compounds of formuale (I)-(IV) can be administered in combination with agents inhibiting VLA-4. Examples of such agents include, Tysabri, Bio-1211. HMR-1031, SB-683698, RBx-4638, RO-0272441, RBx-7796, SB-683699, DW-908e, AJM-300, and PS-460644.

Daily dose of administration of the compounds of the present invention can be repeated, in one embodiment, for one week. In other embodiments, daily dose can be repeated for one month to six months; for six months to one year; for one year to five years; and for five years to ten years. In other embodiments, the length of the treatment by repeated administration is determined by a physician.

The invention is illustrated by the following examples, which are not intended to be limiting in any way.

### EXEMPLIFICATION

### Example 1

### Symadex™ Inhibits Proliferation of B-Cells Following Stimulation with LPS and T-Cells Following Stimulation with Con A in In Vitro Experiments

The activity of Symadex™ was compared to mitoxantrone in several in vitro assays to determine the effect of Symadex™ on several key regulatory systems involved in multiple sclerosis neuroinflammation and antigen presentation.

IL-4 serves as a growth and differentiation factor for B cells, mast cells and macrophages and is a switch factor for synthesis of IgE in mice. It also promotes growth of a cloned CD4⁺ T cell and enhances class II MHC molecule expression and resting B lymphocytes enlargement. In man, CD4⁺ T lymphocytes also produce IL-4, but the human variety has not been shown to serve as a B cell or mast cell growth factor. Both murine and human IL-4 induce switching of B lymphocytes to synthesize IgE. Human IL-4 also induces CD23 expression by B lymphocytes and macrophages in man. IL-4 may have some role in cell mediated immunity.

IL-10 inhibits cytokine synthesis by T_{H}1 cells, blocks antigen presentation, and inhibits the formation of interferon γ. IL-10 inhibits the macophage's ability to present antigen and to form IL-1, IL-6 and TNF-α. IL-10 also participates in IgE regulation. Although IL-10 suppresses cell-mediated immunity, it stimulates B lymphocytes, IL-2 and IL-4 T lymphocyte responsiveness in vitro, and murine mast cells exposed to IL-3 and IL-4. IL-10 may find therapeutic utility by suppressing T lymphocyte autoimmunity in multiple sclerosis and type I diabetes mellitus as well as in facilitating allograft survival.

In this experiment, test compound and/or vehicle were preincubated with human peripheral blood mononuclear leukocyte (PBML, 1×10⁶/ml) in RPMI buffer pH 7.4 for 2 hours. Concanavalin A (Con A, 20 μg/ml) was then added to stimulate the cells overnight in 5% CO₂ at 37° C. IL-4 and IL-10 cytokine levels in the conditioned medium were then quantified using a sandwich ELISA kit. Compounds were screened at 10, 1, 0.1, 0.01 and 0.001 μM.

B-lymphocyte cells isolated from the spleen of balb/c mice weighing 17±1 g were used. Test compound and/or vehicle were incubated with the cells (1.5×10⁶/ml) in the presence of 10 μg/ml lipopolysaccharide (LPS) in AIM-V medium pH 7.4 at 37° C. for 24 hours. [³H]Thymidine (120 nM) was then added for an additional overnight incubation period. Thymidine incorporation was assessed by liquid scintillation counting.

T-lymphocyte cells isolated from thymus of balb/c mice weighing 17±1 g were used. Test compound and/or vehicle is incubated with the cells (4×10⁶/ml) in the presence of 3 μg/mL Concanavalin A (Con A) in AIM-V medium pH 7.4 at 37° C. for 24 hours. [³H]Thymidine (120 nM) was then added for an additional overnight incubation period. Thymidine incorporation was assessed by liquid scintillation counting.

Compounds were screened at 10, 1, 0.1, 0.01 and 0.001 μM.

The results for mitoxantrone and Symadex™ are presented in Table 1. Test compound-induced suppression of cell proliferation by 50 percent or more (≧50%) relative to vehicle control response indicates significant inhibitory activity.

TABLE 1

| | | | SYMADEX ™ | | Mitoxantrone | | |
|---|---|---|---|---|---|---|---|
| | | | % Growth | | % Growth | | |
| | Conc. | | Inhibition | IC50 | Inhibition | IC50 | |
| Mediator | 10 | μM | 105 | 3.33 μM | 108 | 2.96 | μM |
| release, IL-4 | 1 | μM | −9 | | −4 | | |
| | 0.1 | μM | 2 | | 18 | | |
| | 10 | nM | 7 | | 13 | | |
| | 1 | nM | 8 | | 11 | | |
| Mediator | 10 | μM | 99 | 1.2 μM | 102 | 0.759 | μM |
| release, IL-10 | 1 | μM | 39 | | 60 | | |
| | 0.1 | μM | 16 | | 5 | | |
| | 10 | nM | 16 | | 1 | | |
| | 1 | nM | 1 | | 2 | | |
| Cell | 10 | μM | 103 | 0.038 μM | 102 | 7.31 | nM |
| Proliferation, | 1 | μM | 101 | | 102 | | |
| B-Cell + LPS | 0.1 | μM | 80 | | 93 | | |
| | 10 | nM | 38 | | 54 | | |
| | 1 | nM | 0 | | 18 | | |
| Cell | 10 | μM | 104 | 0.014 μM | 103 | 0.032 | μM |
| Proliferation, | 1 | μM | 103 | | 103 | | |
| T-Cell + | 0.1 | μM | 82 | | 80 | | |
| Con A | 10 | nM | 44 | | 19 | | |
| | 1 | nM | 10 | | −1 | | |

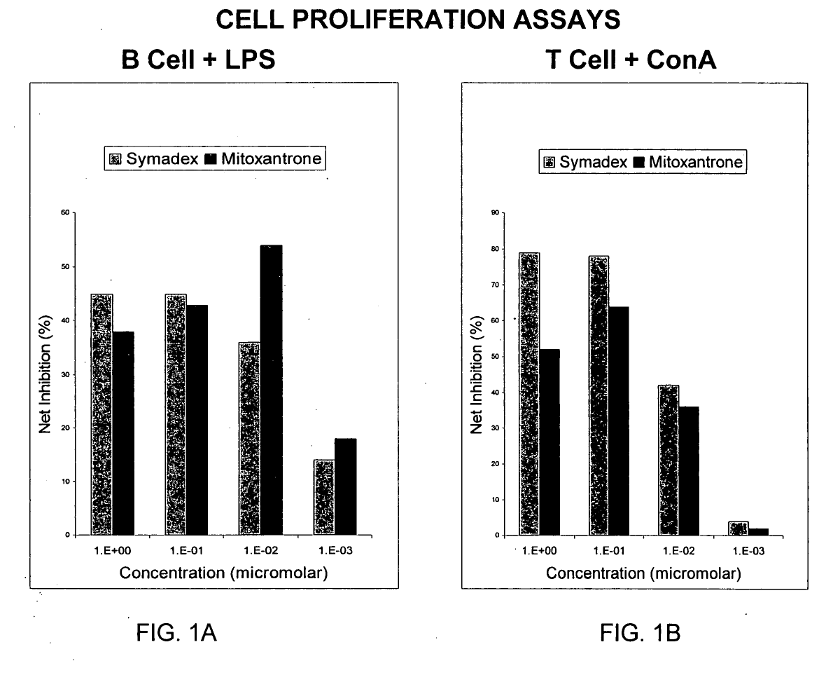

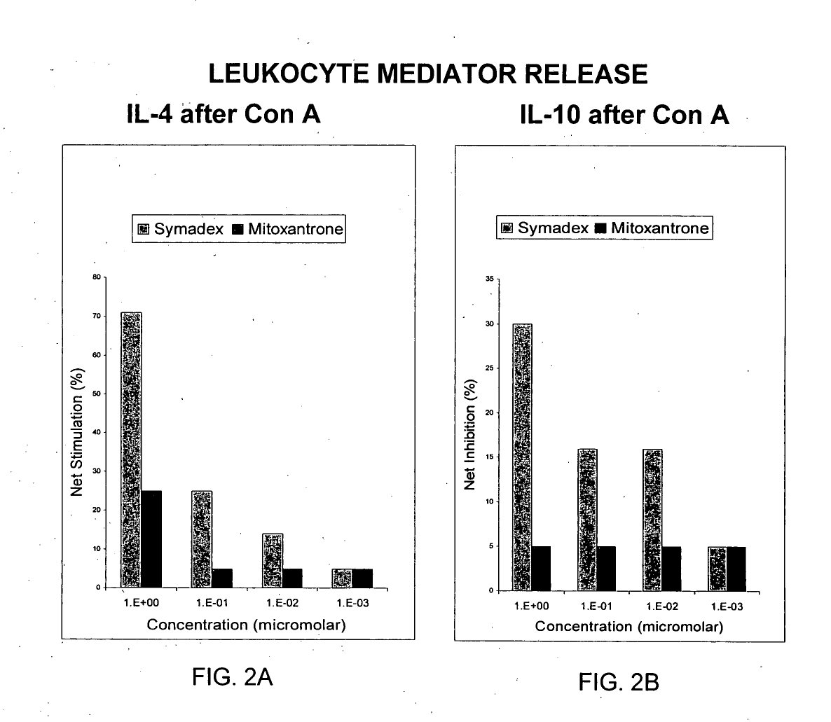

The results presented in Table 1, FIGS. 1A and 1B as well as in FIGS. 2A and 2B indicate that Symadex™ inhibits the release of inflammatory mediators IL-4 and IL-10, both of high importance in neuroinflammatory diseases such as multiple sclerosis. Furthermore, Symadex™ exhibited high level of growth inhibition in in vitro proliferation assays involving B- and T-cells. The activity of Symadex™ was comparable to that of the control compound, mitoxantrone.

### Example 2

### Symadex™ Alleviates the Symptoms of Experimental Autoimmune Encephalomyelitis (EAE), an Animal Model of Chronic Multiple Sclerosis in a Weekly Treatment Cycle for 4 Weeks

One method of showing the utility of the a pharmaceutical compound for the treatment of various conditions associated with multiple sclerosis (MS) is its ability to inhibit effects of Experimental Autoimmune Encephalomyelitis in laboratory animals.

Experimental Autoimmune Encephalomyelitis (EAE) is an animal model for MS, which entails inducing a T-cell-mediated autoimmune disease against myelin basic protein in certain susceptible mammalian species. The EAE model is an appropriate method for studying the inflammation of the brain and spinal cord associated with MS (see Bolton, C. Mult, Scler, 1995; 1(3); 143-9).

In rodents, injection of whole spinal cord or spinal cord components such as myelin basic protein induces an autoimmune response based on the activation of T-lymphocytes. Clinical disease typically becomes manifest around day 8-10 after inoculation, observed as a broad spectrum of behavioral anomalies ranging from mild gait disturbances and tail atony to complete paralysis and death. Weight loss typically occurs. In animals that survive, spontaneous recovery occurs, accompanied by variable recovery of most motor function. Depending on the species, allergen, and methodology used, animals tested by the EAE model may experience a single (acute EAE) or several (chronic relapsing EAE) attacks.

Treatments of EAE come in many structural forms: treatment can be prophylactic or preventative, whereby the therapeutic composition is administered before immunization; treatment can be initiated during the first week of induction; and treatment can be interventious, initiated after clinical symptoms are extent (acute or chronic). Prevention protocols are very common in the literature, treatment after disease is rarer, and treatment after weeks of disease are the most infrequent. The experiments reported herein are in the last classification in which animals in the chronic-progressive (CP) phase with extensive demyelinated plaques are treated. CP-EAE induced by whole CNS in complete Freund's adjuvant is a florid disease with extensive inflammatory and demyelinated changes. As a general philosophy, we believe that successful intervention at later times can better predict effectiveness in the human condition. This is particular relevant to the case of prevention studies, which concentrates on the peripheral immune system, rather than addressing the issue of existing CNS inflammation that is a characteristic of MS.

Methodology

In the present experiment, female juvenile Hartley guinea pigs (225 g) were immunized with homogenized whole CNS (in saline) with an equal amount of complete Freund's Adjuvant and 10 mg added killed M. tuberculosis. The animals (>95%) show clinical signs starting on day 7 post immunization. An acute event of varying severity occurs between day 7 and day 20 followed by a continuous accumulation of clinical abnormality with hind limb paralysis, fecal impaction and incontinence. Table 2 shows the clinical scoring scale. These clinical features indicate inflammation-induced lumbar spinal cord demyelination. A recent survey of previous experiments indicates that taking an animal past day 40, which has a clinical score of “2” for more than 1 week yields a 97% occurrence of demyelinated plaques in the cord.

In these experiments, the immunized animals were nursed until day 40 or day 52 and then treated with 8 mg/kg and 16 mg/kg Symadex™ (intra cardiac), or 20 mg/kg and 40 mg/kg Symadex™ (i.p.) once a week for 4 weeks. Controls were given vehicle. Clinical signs were scored daily and the weights recorded. At the completion of the treatment period, the brain and spinal cord were dissected, formalin fixed and blocked for routine pathological examination of meningeal inflammation, perivascular infiltration (cuffing), parenchymal myelitis and demyelination by a blinded observer using hematoxylin-eosin and solochrome R cyanin stained sections.

Untreated, chronic EAE animals (n=5) were sacrificed on day 40, as well as non-EAE controls (n=5). Following each 10-day treatment interval, five animals from each group were sacrificed (0.25 ml sodium pentobarbital), blood samples were collected for FACS analysis (see below), and the brain and spinal cord dissected and sectioned. Three spinal sections were used, corresponding to lumbar, thoracic and cervical regions of the cord. The brain was cut into five transverse sections; the first three proximal sections were combined in one block, and the last two distal sections in another. Tissues were fixed in 10% formalin and embedded in paraffin. Five micrometer sections were stained with hematoxylin-eosin (H-E) or solochrome-R-cyanin (SCR) and evaluated by a blinded observer in each of the four categories: meningeal inflammation, perivascular infiltration, encephalitis or myelitis and demyelination (Table 2). The combined pathological score represents the total score (out of a potential 20) from all five CNS sections in each animal.

TABLE 2
Pathological scoring scale
M: Inflammatory reaction in the meninges

| | |
|---|---|
| 0: | no changes |
| 1: | perivascular and/or meningeal infiltration by mononuclear cells, 1-3 |
| | vessels involved |
| 2: | 4-6 vessels involved |
| 3: | 6+ vessels involved |
| 4: | dense infiltration of meninges with nearly all or all blood vessels |
| | involved |

P: Parenchymal perivascular infiltrations

| | |
|---|---|
| 0: | no changes |
| 1: | 1-3 parenchymal vessels infiltrated in Virchow-Robin spaces |
| 2: | 4-6 vessels involved |
| 3: | 6+ vessels involved |
| 4: | virtually all vessels involved |

E: Encephalitis or myelitis

| | |
|---|---|
| 0: | no invasion of the neural parenchyma; microglial or inflammatory |
| | cells invading neural parenchyma |
| 1: | a few scattered cells |
| 2: | invasion by cells from several perivascular cuffs |
| 3: | large areas of neural parenchyma involved |
| 4: | virtually the entire section is infiltrated |

D: Demyelination, remyelination and myelin debris

| | |
|---|---|
| 0: | no demyelination |
| 1: | single focus on subpial demylination or myelin debris |
| 2: | several small foci of demyelination |
| 3: | one large confluent area of demyelination |
| 4: | several large confluent areas of demyelination |

To quantify the abnormalities observed in the spinal cord, sections stained with H-E were divided into 12 representative pie-shaped areas. In each area, the number of cells within a 0.12-mm² field of view was counted using Sigma Scan Pro image analysis software (SPSS), and the combined mean number of cell in all 12 areas was calculated for the whole spinal cord (36 fields of view per animal). Note that as all cell nuclei were counted, the number of cells may include neurons and glial cells in addition to infiltrates. Hence, the cell count in non-EAE animals served as a baseline.

Results

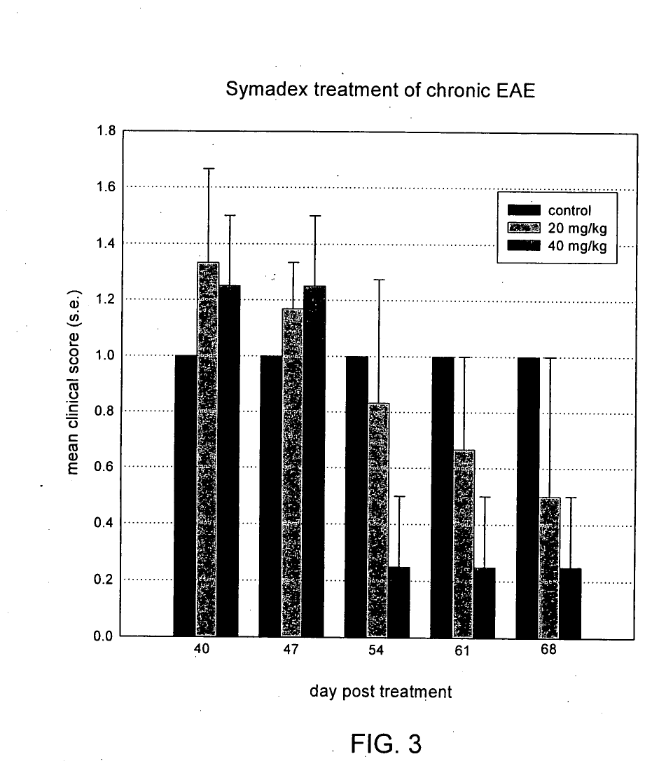

Symadex™ produced a profound and substantial change in the clinical progress and pathological findings when given at 20 and 40 mg/kg (i.p.). FIG. 3 illustrates the mean clinical score of the animals at the indicated day thereafter. The treated animals all showed some degree of clinical recovery with the 40 mg/kg group reaching recovery within 2 weeks of the start of treatment.

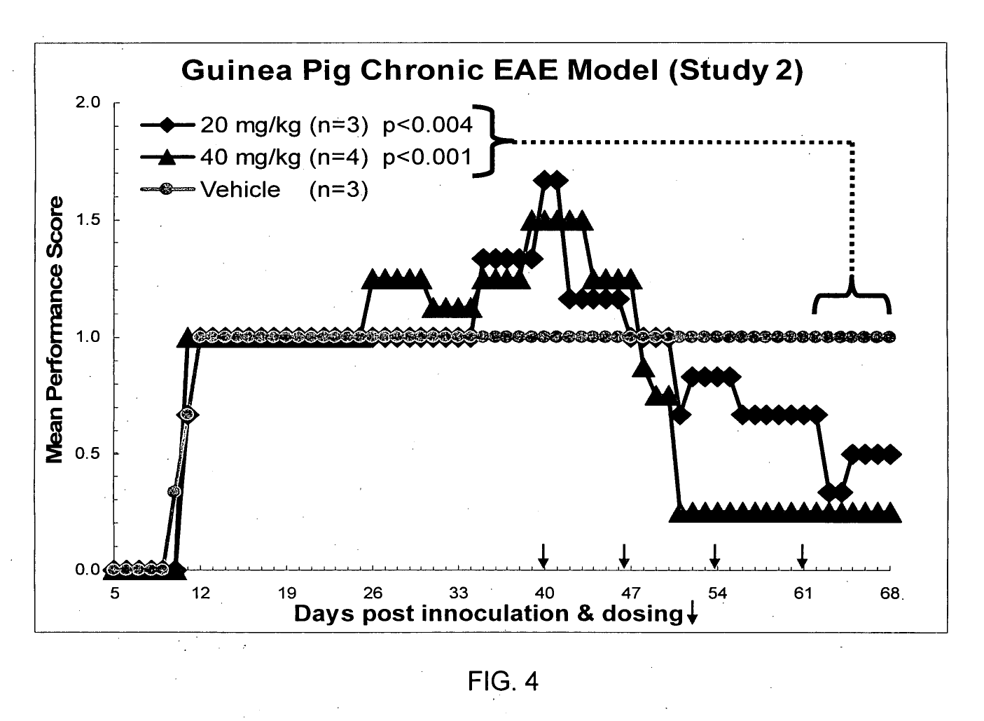

The longitudinal course of recovery from disease is further illustrated in FIG. 4. In this experiment, the vehicle controls showed a steady course of disease. Treated animals, in both the 20 and 40 mg/kg cohorts entered the study presenting disease of greater severity than controls, a coincidental circumstance attributed to the phenomenology of randomization prior to assignment of a treating group. As indicated by the arrows, treatment began on day forty and progressed for a total of four doses. At the end of the treatment, both treated cohorts showed significant disease improvement to levels significantly below control, despite having entered the study at a disease level well above control. Statistical significance, even with 3 and 4 animal small cohorts showed p values supporting the hypothesis that treatment afforded a statistically different outcome over control. These were determined by non-parametric comparisons by the Mann-Whitney or Wilcoxon rank-sum test for difference in medians. The significance values were 0.001 and 0.004 for the 40 and 20 mg/kg cohorts, respectively.

The pathological findings were most unusual. The scores for meningeal inflammation and perivascular infiltration were more severe in the treated groups than in vehicle controls (data not shown). However, we observed two highly significant findings: existing lesions had a profound loss of cells (data not shown) and we observed myelin pallor previously attributed to remyelination (data not shown). The latter observation is consistent with permissive remyelination of the CNS due to removal of the inflammatory cells.

Demyelination is a key pathological feature of the MS lesion. Not only does this alter electrical response of the axon, current thought suggests that prolonged demyelination can result in permanent axonal damage and death. Thus neurodegeneration is also a key component of the MS pathological milieu. In this regard, Symadex™ has proved to be effective in permitting endogenous remyelination even after a period of disease progression that reached 97% spinal chord demyelination in this chronic-progressive model. It appears to permit this CNS recovery by reducing the inflammation in existing lesions. After prolonged Symadex™ treatment, it is possible to observe chronic demyelinated plaques that have virtually no remaining inflammatory cells and some of these lesions show the myelin pallor indicative of remyelination (called a shadow plaque in MS). Prevention of new T-cell infiltration by deletion of these cells or down regulation or inhibiting cell trafficking would prevent the recruitment of further macrophages to an inflammatory lesion. As a consequence, the immune cells in the lesions die by apoptosis and the lesions are left relatively free of infiltrates. Removal of the cytokine, and ROS-mediated tissue toxicity of macrophages would allow the CNS reparative mechanisms to become active and remyelination is observed. It is thus likely that Symadex™ has an effect on the peripheral immune system, although a direct effect on CNS inflammation cannot be ruled out.

The continued presence of large inflammatory cuffs and meningeal inflammation scores that were higher than control is consistent with the continued production of immune competent leukocytes which accumulate around CNS vessels, but do not traffic into the parenchyma.

### Example 3

### Symadex™ Alleviates the Symptoms of Experimental Autoimmune Encephalomyelitis (EAE), an Animal Model of Chronic Multiple Sclerosis in a Weekly Treatment Cycle for 4, 6, 8 and in a 4 Week on Drug-4 Week Off Treatment Cycle

The experiment described in Example 2 was extended to a larger cohort and longer treatment cycle with several objectives in mind. In addition to corroborating the initial findings, a concerted effort was directed at also demonstrating the extent and durability of response, including the effect after drug withdrawal, and to document the impact of drug treatment on immune function in order to uncover any signals of impending impairment or toxicity.

Following disease induction, as previously described, the animals were randomized into 5 five cohorts, one vehicle control and 4 treatment cohorts. Animals in the treatment cohorts were administered study drug intraperitoneally at 20 mg/kg (Symadex™dihydrochloride trihydrate) once a week for 4, 6 and 8 weeks, with an additional cohort treated once a week for 4 weeks and observed for an additional 4 weeks of treatment with vehicle solution (saline) rather than with drug.

The only significant protocol deviation from the method of Example 2 was that the pool of immunized animals was culled of animals presenting with a disease severity greater than 2 and randomized so that the mean disease severity of each cohort was matched in the severity score range of 1 to 1.5. This measure was invoked in order to avoid the chance circumstance, observed in Example 2, that animal selected for treatment should start treatment with a more severe presentation than the corresponding vehicle controls.

All treated animals showed statistically significant improvement in disease. That is, their symptoms of paralysis attributable to the demyelinating progression of inflammatory cell assault on nerve chord parenchyma, were reduced close to baseline, pre-disease levels. These results are evident by mere inspection of the disease course plots and also proved to be highly significant by non-parametric, rank-order statistical analysis.

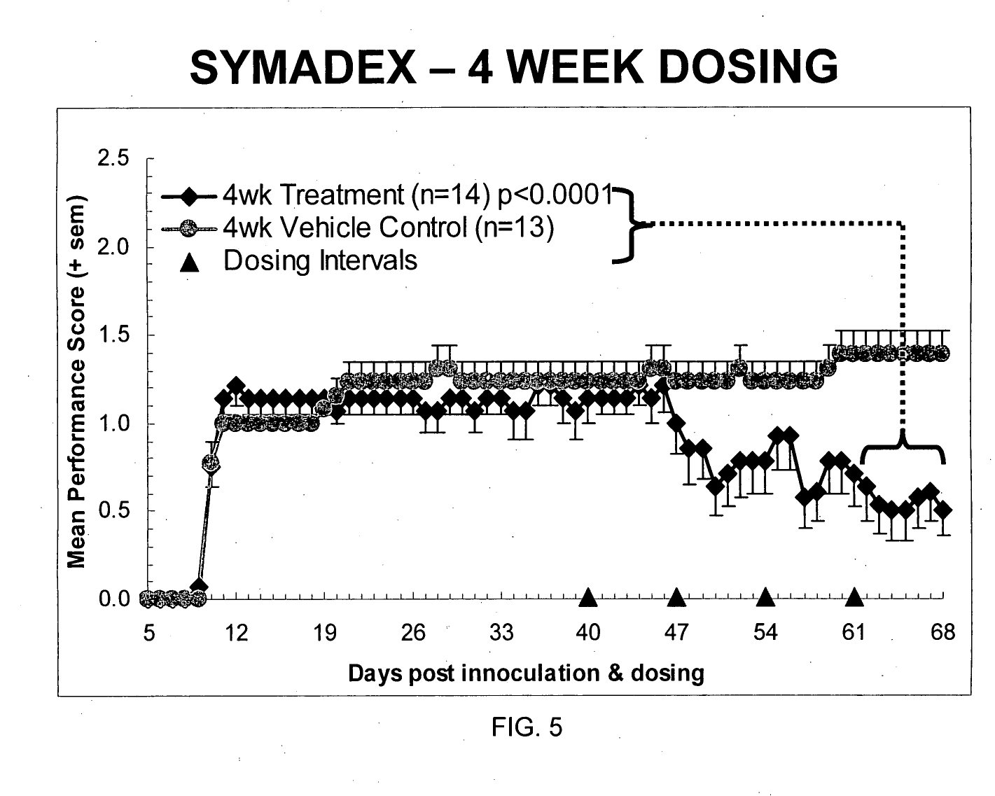

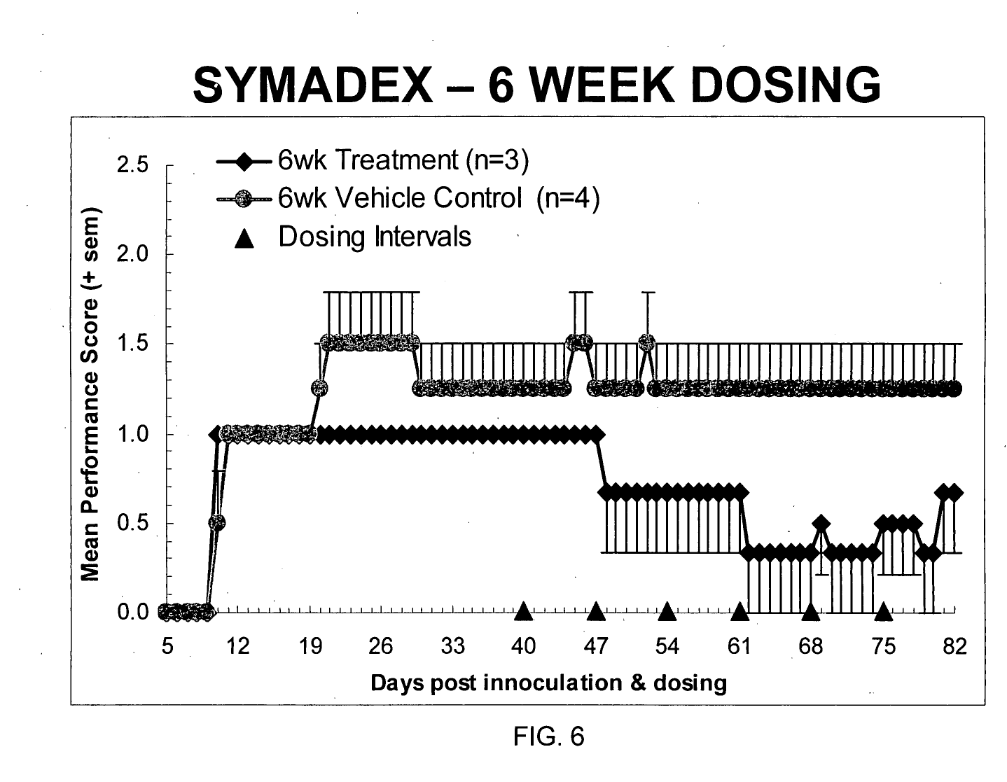

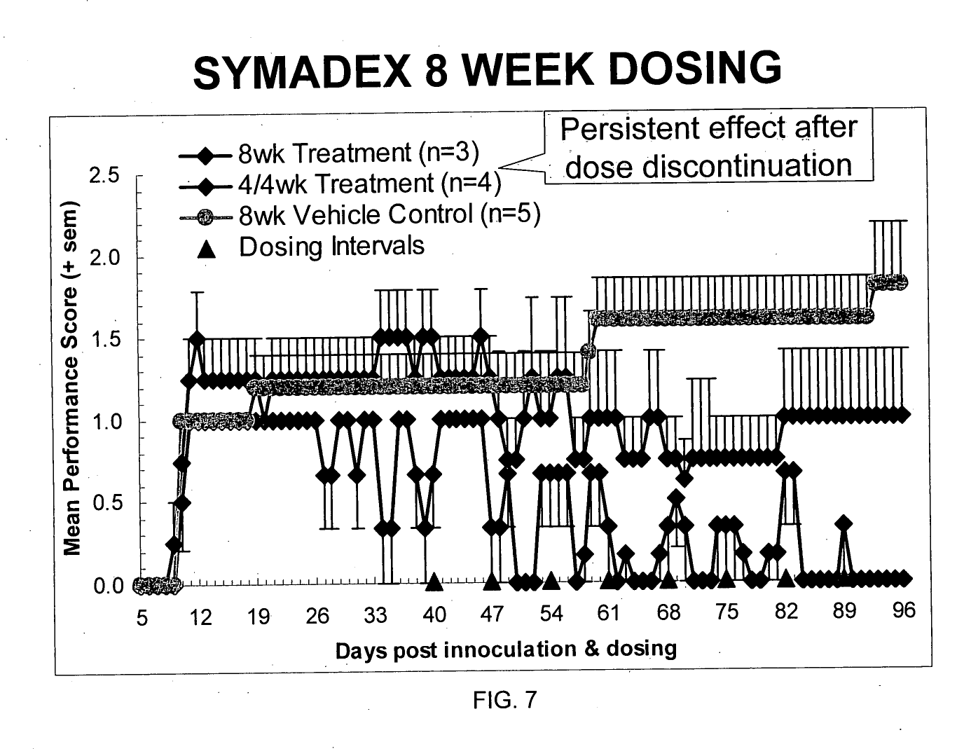

The 4-week treatment cycle result (n=14), as shown in FIG. 5, demonstrates a declining trend in clinical severity score to a mean level 0.7 from a starting disease presentation mean of 1.3. This change is statistically different from control (n=13), with a p value on differences in median of 0.0001. A similar dose response is demonstrated in FIG. 6, which describes the 6 week treatment course. Again, disease improved from a mean severity score of 1.3 for control (n=4) down to 0.3 (n=3) for the treatment cohort, with a statistical significance p value of 0.009. FIG. 7 presents the 8 week treatment cohorts. Attention is called to the vehicle control, whose disease shows progression towards greater severity, and hence greater paralysis attributable to demyelination, with a gradual rise after 4 weeks from a level of 1.3 to 1.7. In contrast, both treatment cohorts, show progressive disease amelioration, indication reversal of demyelination. In the case of animals treated with 8 consecutive doses, the trend towards normal, pre-disease clinical scores is initially variable but converges on baseline at the end of the treatment course. Despite the interindividual heterogeneity in recovery profile, the full rank-order analysis shows the difference between treatment (n=3) and control (n=5) to be significantly at a p value of 0.0006. The cohort with treatment discontinued treatment after 4 weeks, remained in stable disease at the time of discontinuation, also at a level significantly different from control, with a p value of 0.002.

The results of this study are shown in FIG. 7. First, the therapeutic response shows a graded temporal response against control. Sick animals become progressively healthier in proportion to the weekly duration of response, while control animals progress irreversibly towards complete neurological dysfunction, whose root cause is irreversible demyelination. Second, the cumulative pharmacodynamic effect of drug treatment is durable, because treated animals remain in stable condition, commensurate with their degree of treatment, while untreated controls sustain an accelerating progression in disease. This is a particularly noteworthy observation in light of the effects obtained with the most promising recent therapy for progressive MS, namely the α4 integrin antagonists like Tysabri and its small molecule ligand equivalents, as described by Piraino P. S. et al, J. Neuroimmunology, 131:147-159, 2002). When treatment is discontinued in the same guinea pig EAE model of MS, the animals in the treatment cohort revert within 7 days to the disease level of untreated controls, with no evidence of a protracted beneficial pharmacodynamic effect.

The contrasting result between the therapeutic effects of Symadex and the α4 integrin antagonists, as well as between Symadex and other therapies that interdict T-cell mediated inflammatory responses, is that Symadex does not exert its action via the activation and recruitment of inflammatory cells. The histopathology of spinal chord from animals sacrificed at periodic interval throughout the time course of disease recovery show accumulation, rather than diminution, of inflammatory cells in blood vessels and perivascular cuffs, as noted in Example 2. Yet, these cells are apparently blocked from transmigrating beyond the basement membrane of parenchyma, suggesting a block via mechanisms that could involve: cell adhesion, motility, and extracellular matrix remodeling.

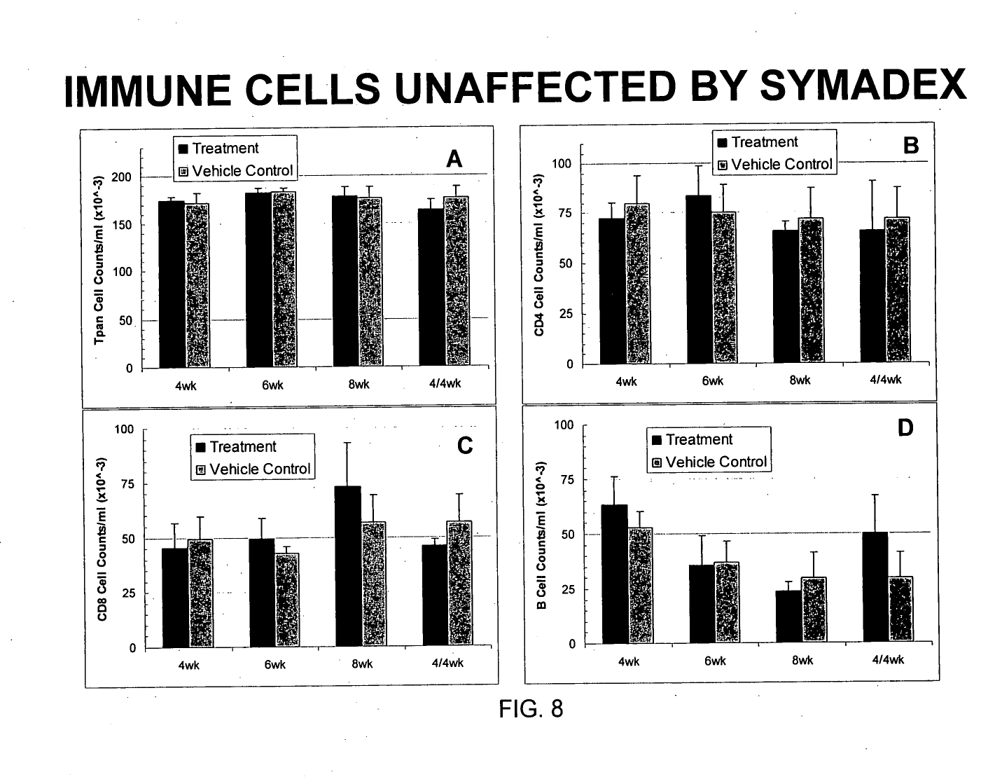

It is demonstrable as a differentiating, and unexpected, feature of Symadex's mode of action, when compared to corticosteroid, interferon, and integrin antagonist therapies that there are no changes in T-cell populations or in T-cell sub-type ratios. In the instant example, as shown in FIG. 8, Panel A, no difference is observed between any treatment cohort and vehicle control with respect to total T-cell counts. Neither are CD4 or CD8 T-cell populations modulated by Symadex™ treatment, as shown in panels B and C. Even B-cells show no significant deviation from controls, although there appears to be a declining trend across the board as a function of disease or treatment duration. Like the cytotoxic therapies for MS, e.g. mitoxantrone and cyclophosphamide, for example, Symadex™ may exert a transient diminution in B-cell counts in stimulated cell cultures, according to the evidence presented in Example 1, but the effect is not evident on prolonged exposure in vivo. This observation further reinforces the notion that Symadex™ acts by a novel mechanism that will not deplete the immune system nor diminish host defenses against antigen presenting pathogens. Such a property would be highly desirable and advantageous in any therapy intended for the treatment of chronic conditions or acute conditions, such as MS flair ups, in otherwise healthy subjects.

### Example 4

### Symadex™ Reverses the Symptoms of Experimental Autoimmune Encephalomyelitis (EAE), an Animal Model of Chronic Multiple Sclerosis after Two Doses Administered 72 Hours Apart

Analysis of the time course of disease recovery upon treatment with Symadex™ on a weekly basis reveals a two to three day periodicity in the amelioration of disease. This phenomenon is particularly evident in the 8 week treatment test cohort shown in FIG. 7. Individual animals in the cohort can be seen to respond differently so that the pharmacodynamic response on average presents itself in a “saw-tooth” pattern between successive dosing intervals. In tracing the records of specific animals within the cohort, it appears that some recover transiently and show disease improvement for 2-3 days after receiving a dose and then revert to a higher disease score. The overall trend leads to disease improvement over 8 weeks, but the “saw-tooth” response phenomenon raises questions about the temporal spacing of treatments that optimally achieve a smoother reversal of disease symptoms.

In order to test the possibility that a more frequent dosing schedule would offer a more rapid resolution of disease symptoms, an experiment was performed to match the dosing cycle to the observed periodicity of response. Accordingly, the method of Experiment 2 was applied to a cohort of animals and controls, which were allowed to reach the chronic phase of disease at 30 days post immunization. Six animals with a disease score of 1 were selected and half were treated with 20 mg/kg Symadex™ administered intraperitoneally. Two dose were given 72 hours apart to 3 animals. Three animals served as vehicle controls.

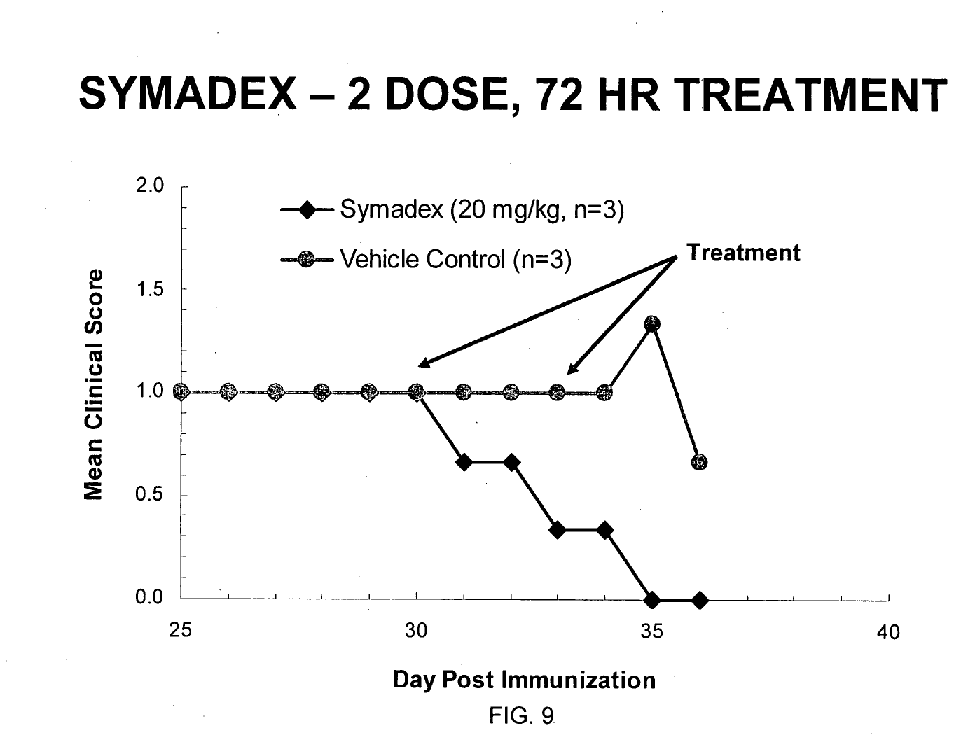

As shown in FIG. 9, the Symadex™ treatment on a 72 hour schedule reversed the progression of disease and restored baseline clinical scores with just 2 doses, while disease progression continued in the control cohort. The difference is statistically significant by a p value of 0.002 by the rank-sum test.

This experiment demonstrates that a more frequent dosing of Symadex™ can be tailored to match the particular balance between drug residence time, the temporal properties of the assault by inflammatory cells on myelin, and the intrinsic processes of permissive remyelination. It would be reasonable, therefore, to expect that combinations of dosing regimens can be applied first to accelerate recovery from disease, by more frequent or intense schedules of drug delivery, and then to maintain the beneficial effects of inflammatory cell blockade with less frequent, but, periodic, booster doses. The “saw-tooth” patterns of treatment and disease reversion evidenced in FIG. 7, in Example 3, suggested that the effect of Symadex™ can be attenuated over a 2-3 day interval between doses. This experiment confirms that the efficacy of Symadex™ can be re-enforced through more frequent dose administration.

### Example 5

### Symadex™ Alleviates the Symptoms of Experimental Autoimmune Encephalomyelitis (EAE), an Animal Model of Acute Multiple Sclerosis Upon Daily Dosing Over the Initial Course of Disease Induction

As described in Example 2, the EAE model in the guinea pig is biphasic. After the myelin basic protein insult on initial immunization, the typical clinical pattern of neurological impairment begins with acute signs of disease day 9 post immunization. Clinical onset results in weight loss, hind limb weakness and an abnormal righting reflex. The severity of these symptoms peaks over 6-7 additional days followed by a short duration transient and partial resolution by day until day-20, when the disease course changes to a steady progressive decline, from which there is no clinical recovery.

As an important extension to the utility of Symadex, its therapeutic effect at this earlier stage of disease presentation was examined. The experiment was further designed to build on the results of Example 4, which suggested that more frequent dosing affords more rapid and unidirectional symptom resolution. Since the acute phase of EAE also mimics active, but not necessarily progressive disease, as would be expected to be the case in human subjects with remitting-relapsing multiple sclerosis, the experiment was further designed to test the efficacy in comparison to mitoxantrone. This later drug, as indicated earlier, is an approved therapeutic agent and had served as the starting point for the chemical evolution of what became the Symadex™ molecule minus the toxicophores known to be causative agents for cardiotoxicity.

Accordingly, three randomized cohorts of guinea pigs with EAE induced by the method of Example 2, were treated, respectively, with 6 mg/kg of Symadex™ (full salt hydrate) and 0.35 mg/kg of mitoxantrone. Animals were treated daily, by intraperitoneal injection, for 15 days, starting on day 7 post immunization. Controls were treated with vehicle. We reasoned that 15 consecutive, 6 mg/kg doses of Symadex™ would represent a level of drug exposure that would be commensurate with the “20 mg/kg every 72 hours” regimen in Example 4 and consistent with a mid level exposure between the 20 mg/kg and the 40 mg/kg schedule given weekly, in Example 2. The mitoxantrone dose was selected to reflect a typical high dose given to rats or mice by daily dosing in the prior art, but allometrically scaled to the guinea pig.

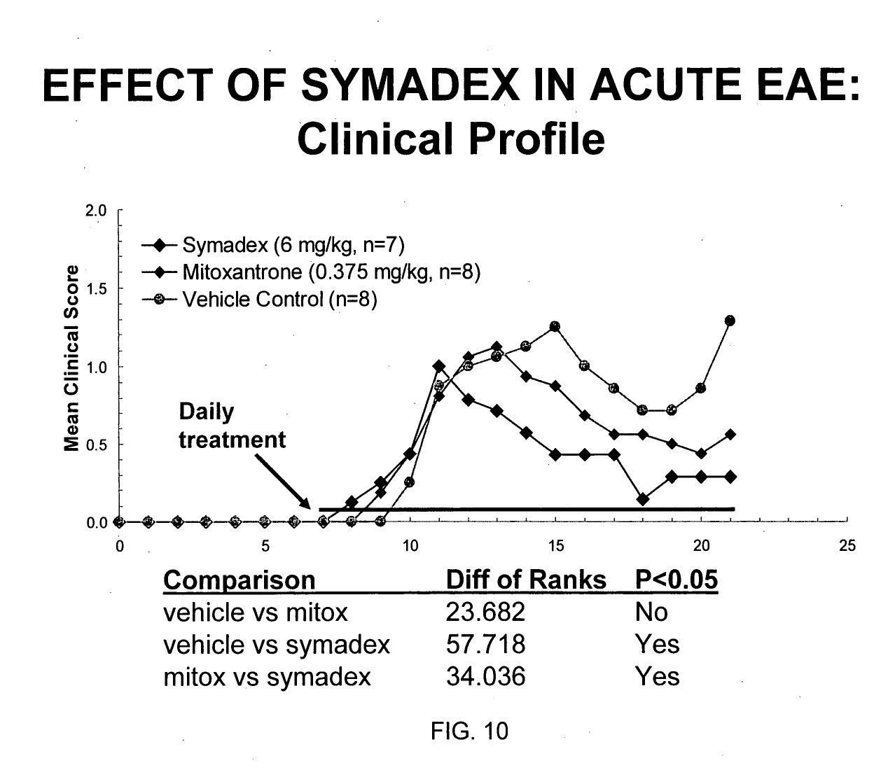

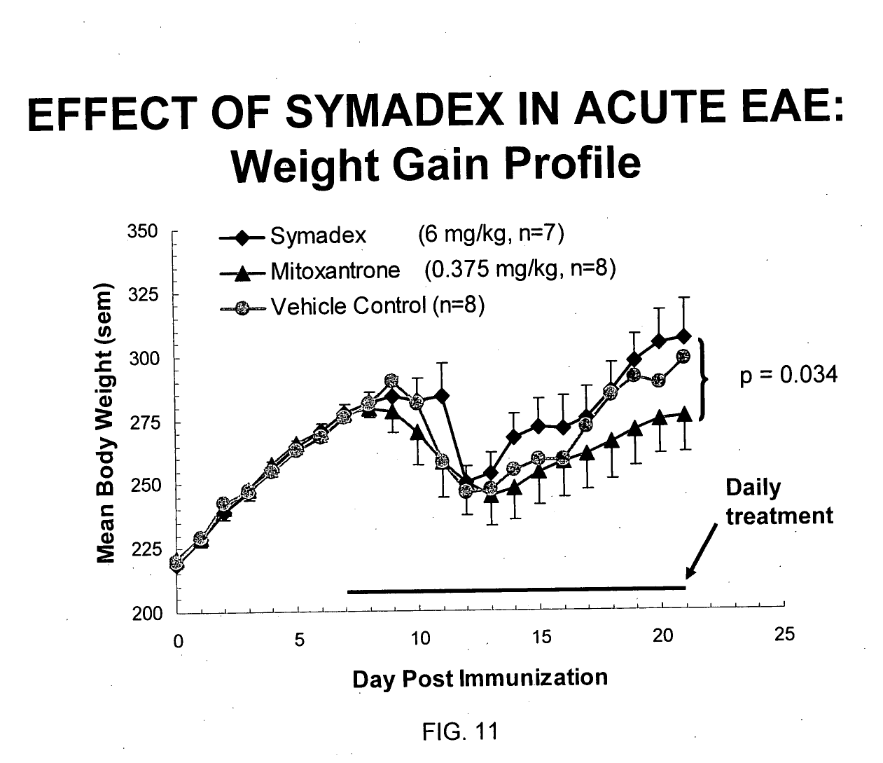

As can be appreciated from the results presented in FIG. 10, the cohorts treated with either Symadex™ or mitoxantrone present a consistently different trend than the vehicle controls. In the controls, the onset of disease is followed by a steady increase in clinical score until day 15, followed by the characteristic short term reversion and a second climb towards higher disease severity by day 20. In the case of both mitoxantrone and Symadex, the initial rise in neurological impairment is arrested, and all animals continued on a course of recovery towards basal levels throughout the dosing period. However, statistical analysis by Mann-Whitney rank-sum tests indicates that the therapeutic effect of Symadex™ against both control and the performance of mitoxantrone reaches statistical significance with a p value below 0.05. The difference in median scores between the performance of mitoxantrone and control did not reach statistical significance. FIG. 11 shows the weight gain profile, a sensitive indicator of general health status in guinea pigs. Acute EAE disease onset triggers a rapid weight loss from which there develops a steady recovery after day 15. Control animals and Symadex™ treated animals regain the ability to add weight every day, which is the norm for guinea pigs when healthy and prior to the onset of severe, chronic disease. However, under the circumstances of this experiment, mitoxantrone may have alleviated the acute clinical symptoms of EAE at a lower relative dose but it also impairs weight gain, a sign of generalized toxicological response to a drug that is a potent and broad spectrum cytotoxic. Comparison of the weight gain profiles between vehicle controls and Symadex™ did not show statistical significance by the Mann-Whitney rank-sum test, whereas the difference between Symadex™ and mitoxantrone reached statistical significance with a p value of 0.034.

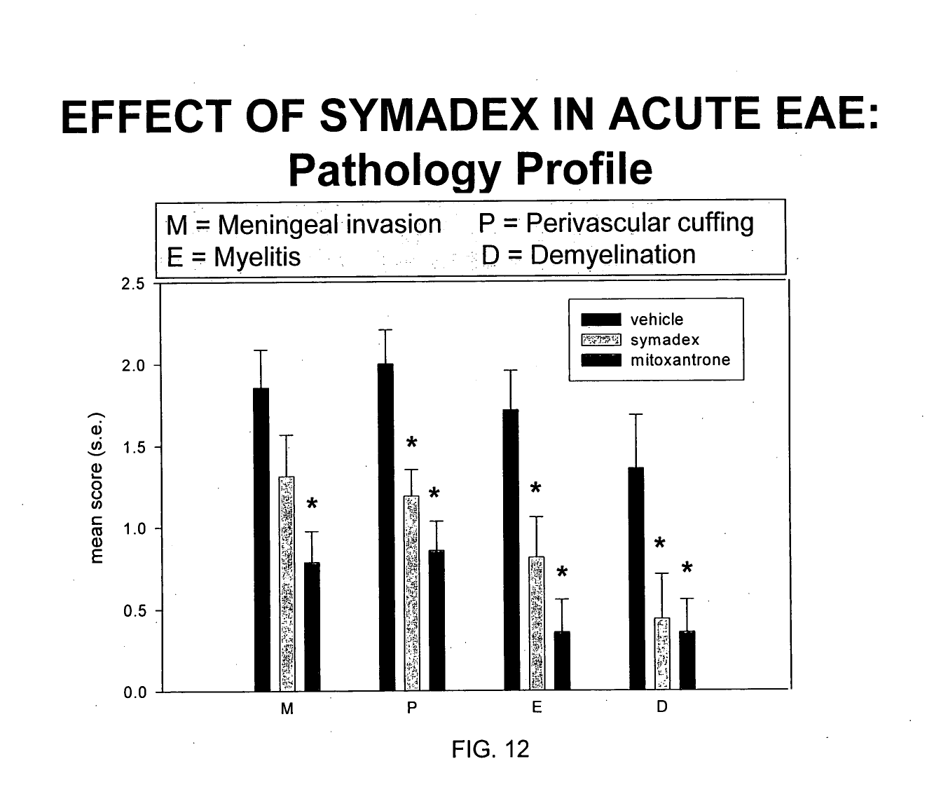

These results confirm that Symadex™ modifies the presentation of EAE throughout the course of active disease, both at the early acute and at the chronic phase without imposing a deleterious cytotoxic load. An analysis of the pathophysiology, shown in FIG. 12, confirms the earlier observations, by the histological methods described in Example 2, that Symadex™ arrests the invasion of parenchyma by inflammatory cells. It does so with a significant difference from the mode of action of mitoxantrone and drugs in the mitoxantrone class which are immunosuppressive. While the outcome may be the same in terms of the effect of both drugs on reducing the perivascular cuffing (P), myelitis (E) and demyelination (D), as judged by statistically significant differences (p value<0.05) from control, Symadex™ and mitoxantrone do not show the same action on meningeal inflammation (M). Mitoxantrone is an immunosuppressant that blocks activation and recruitment of inflammatory cells given the statistically significant reduction in meningeal inflammation (M). Symadex™ does not, although the therapeutic outcome according to the remaining three histopathological assessments is essentially similar.

These findings are relevant to the human disease circumstance, because it is considered highly beneficial to effect treatment of MS conditions without impairing the host's ability to mount an immunological, and hence inflammatory response, against adventitious infections. It terms of response to cytotoxic agents, which might impair gastrointestinal function and nutritional maintenance, the lack of negative effects on normal growth and weight gain in these guinea pig experiments points to another safety advantage that may accrue to Symadex™ therapy. The cumulative 15 day dose of Symadex™ for treatment of acute, active disease is 90 mg/kg. Allometric scaling to human dosimetry levels yields a corresponding human dose of 540 mg/m² body surface area (as free base), which has been shown to be a safe and well-tolerated single dose, and is lower than the 640 mg/m² dose which is indicated as a repeat dose every three weeks. Allometric scaling of the mitoxantrone dose, on the other hand, represents a total human equivalent dose of 45 mg/m². Since mitoxantrone is used in the treatment of MS on a three month dosing cycle at 12 mg/m², this represents the total dose for a year's worth of treatment. Thus, the experimental findings in this comparative example on the relative efficacy of Symadex™ versus mitoxantrone suggest that in humans a single dose of Symadex™ should show a similar, if not greater, therapeutic benefit as an year's course of mitoxantrone.

### Example 6

### Symadex™ Alleviates the Symptoms of Collagen Antibody Induced Arthritis in the Mouse, an Animal Model of Rheumatoid Arthritis and Autoimmune Disease

Rheumatoid Arthritis (RA) is an autoimmune disorder characterized by the chronic erosive inflammation in joints leading to the destruction of cartilage and bones. Several disease modifying antirheumatic drugs (DMARDS) are used in the treatment of RA. Currently, the two most important DMARDS are inhibitors of tumor necrosis factor α (TNF-α) and methotrexate (MTX). One method for demonstrating the utility of a pharmaceutical compound for the treatment of various conditions associated with RA is its ability to inhibit the induction of arthritis by collagen monoclonal antibodies (mABs) in mice.

Collagen-induced Arthritis (CIA) is an experimental autoimmune disease that can be elicited in susceptible strains of rodents (rat and mouse) and nonhuman primates by immunization with type II collagen, the major constituent protein of articular cartilage. CIA manifests as swelling and erythema in the limbs of the mouse. This model of autoimmunity shares several clinical and pathological features with rheumatoid arthritis (RA) and has become the most widely studied model of RA. CIA in the mouse model was first described by Courtenay et al. in 1980 (Courtnay, J. S., Dallman, M. J., Dayman, A. D., Martin A., and Mosedale, B. (1980) Immunisation against heterologous type II collagen induces arthritis in mice. Nature 283, 666-668). Like RA, susceptibility to CIA is regulated by the class II molecules of the major histocompatibility complex (MHC), indicating the crucial role played by T cells.

Methods

Groups of 3 BALB/c strain mice, 6-7 weeks of age, were used for the induction of arthritis by monoclonal antibodies (mABs) raised against type II collagen, plus lipopolysaccharide (LPS). A combination of 4 different mABs (D8, F10, DI-2G and A2) totaling 4 mg/mouse was administered to the animal intravenously on day 0, followed by intravenous challenge with 25 mg/mouse of LPS 72 hours later (day 3). From day 3, test substance and vehicle were each administered orally once daily for 3 consecutive days. For each animal, volumes of both hind paws were measured using a plethysmometer with water cell (12 mm diameter) on Days 0, 5, 7, 10, 14 and 17. Percent inhibition of increase in volume induced by mABs+LPS was calculated by the following formula:
Inhibition (%): [1−(Tn−T0)/(Cn−C0)]×100%
Where:

C0(Cn): volume of day 0 (day n) in vehicle control

T0(Tn): volume of day 0 (day n) in test compound-treated group

Reduction of edema in the hind paws by 30% or more is considered significant.

Results

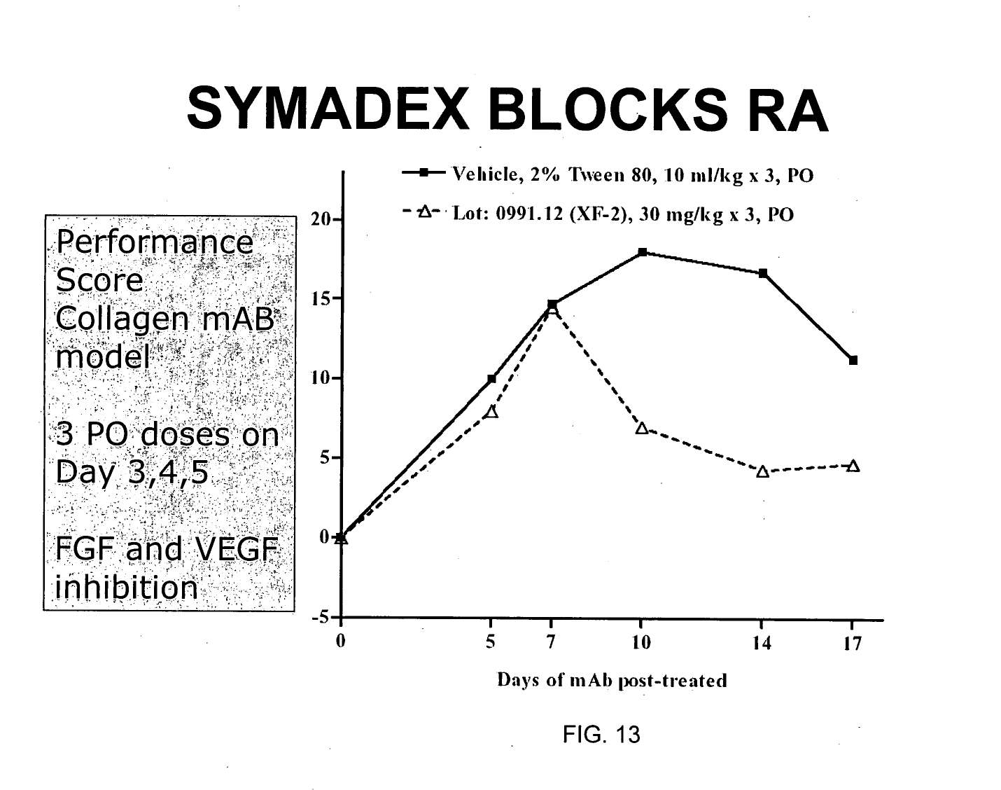

To monitor the onset of CIA, the volume of the two hind paws of mAB treated mice were measured. In the control (vehicle) treated animals the paws quickly became inflamed with a 42% increase in volume on day 5, the maximum volume was observed on day 10 and then the swelling began to subside. As shown in FIG. 13, in the Symadex™ treated group, the initial swelling on day 5 was slightly lower then control (32% vs. 42%) and significantly less inflammation (measured by paw volume) was observed on day 10 (29% vs. 75%), day 14 (18% vs. 70%) and day 17 (19% vs. 47%). The difference in means by paired t-test and in medians by non-parametric Mann-Whitney rank-sums all show p values lower than 0.01.

Conclusion

Symadex™ demonstrated significant anti-arthritic activity in the mouse CIA model, with significant anti-inflammatory activity on day 10 (61% inhibition), day 14 (74% inhibition) and day 17 (59% inhibition). These findings are relevant in the context of prior example on EAE and autoimmune disease in general because they exemplify the efficacy of Symadex™ via an unexpected mechanism. The collagen antibody model of rheumatoid arthritis is significant because it by-passes the primary inflammatory insult of antigen presentation. Classical anti-inflammatories like the corticosteroids and anti-folates like methotrexate alleviate the consequence of autoimmune inflammatory diseases by suppressing the primary events of inflammatory cell activation and recruitment. The antibody induced model generates the symptoms of disease that present in the later stages of the autoimmune response, after activated cells become invasive into cartilage, having extravasated and transmigrated, as would be the case in MS during a prolonged assault on parenchyma.

Methotrexate, a benchmark therapeutic agent, has been shown to yield diminishing benefit in antibody induced models, which are intrinsically less dependent on T-cell activation than on their trafficking and migratory properties. The work of Lange et al. can be cited in this context (Annals of Rheumatoid Disease 64:599-605, 2005). By contrast, Symadex™ appears fully active in this model. The results presented in this example are especially relevant to the treatment of human subjects, because the therapeutic effect was obtained by oral administration. In the era of injectable biologics, such as blocking antibodies, the addition of an effective, non-immunosuppressive therapy via the oral route is particularly desirable.

### Example 7

### Symadex™ Downregulates Otherwise Overexpressed Target Mechanisms of Inflammatory Cell Adhesion, Cell-Surface Signaling, and Cell Proliferation

To explore the effect of Symadex™ treatment on gene expression, microarray experiments were performed.

Two colorectal cancer cell lines (HT29 & HCT116) were chosen for study, whose behavior as rapidly proliferating invasive cells could be generalized to many other such cell types from different tissue origins. The two lines were immortalized colon carcinomas. Their gene expression patterns are known to mimic the behavior of neuro-enteric cells and therefore provide an appropriate simulation of the kinds of regulatory patterns that would be found in cells of similar epithelial or endothelial origin. Cells with these ontological roots are also suitable models for the kinds of autoimmune and inflammatory susceptibilities that are common in tissues of neuroenteric origin, such as those in which inflammatory bowel disease would present itself.

Attention is drawn here to the exhaustive studies, using differential gene expression arrays (Zhang J. et al., “Neural system-enriched expression: relationship to biological pathways and neurological diseases”, Physiol. Genomics 18:167-183, 2004) which have documented the redundancies and commonalities of gene expression patterns in both the central nervous system and in anatomically unrelated tissues. For example, Zhang and colleagues, whose teachings are incorporated here by reference, profiled the expression products of 8,734 genes in 10 regions of the nervous system and in 30 peripheral organs. Their analyses reveal that approximately 70% of the genes relevant to nervous system diseases are also expressed in multiple tissues, including those of epithelial origin and in peripheral blood. These investigators suggest further that the profiling of genes implicated in nervous system diseases but sourced from various peripheral tissues, where easier sampling can be obtained, will aid the development of better mechanistic understanding about those diseases. Hence, the use of colon cells in gene expression studies as a model paradigm for understanding the effect of a drug on pathways common to those cells and nervous system tissues is experimentally justifiable.

Accordingly, the specific studies to document the mechanism of action of the compounds in the instant invention, were conducted as follows using the preferred imidazoacrinidone composition, referred to hereinafter as Symadex™.

Cells were grown in the presence of Symadex™ at the GI50 concentration (0.68 and 0.21 μMolar, for the HT29 and HCT116 cell lines, respectively), and harvested along with untreated control fractions after 1, 8 and 48 hrs. of exposure. Frozen cell pellets were lysed in triplicate and total RNA isolated by purification over spin columns (all reagents from Ambion). After QC acceptance for purity, total RNA was converted to cRNA by linear amplification and 10 μg samples were applied to CodeLink Human Whole Genome Bioarrays (GE Healthcare and GenUs Biosystems).

Arrays were processed in triplicate and comparisons made after robust statistical analysis of replicate variability. Genes (including ESTs) were considered to be differentially expressed if a change from baseline could be demonstrated as significant by T-test (p<0.05, α=0.025), using CodeLink Expression Analysis (GE Healthcare) and GeneSpring (Silicon Genetics) software. False discovery rates and representation in standardized gene ontologies/pathways were then determined by filtering the “fold” changes in expression with open access software packages, EASE and GoMiner, and with Pathways Analysis (Ingenuity Systems). Functional annotations were then explored further in the MedMiner literature search environment.

Over the interval sampled in the 24 hour test incubation, 271 down-regulated genes were significantly represented in both cell types, from within an array of 55,000 gene fragment accessions. A listing of these is shown in Table 3, in which the first column data presents the fold change against control, the second column cites the gene symbol, the third column cites the Genbank Accession, and the fourth column provides an abridged description of the gene's function.

TABLE 3
Significantly Downregulated Genes by the Action of Symadex ™

| MEAN | | | |
|---|---|---|---|
| FOLD CHANGE | | | |
| OVER | GENE | GENBANK | |
| CONTROL | SYMBOL | ACCESSION | DESCRIPTION |
| −17.00 | ACTA2 | AL713608 | actin, alpha 2, smooth muscle, aorta |
| −9.43 | ACVRL1 | NM_000020 | activin A receptor type II-like 1 |
| −2.07 | ACYP1 | AA664719 | acylphosphatase 1, erythrocyte (common) type |
| −21.74 | ADCYAP1 | NM_001117 | adenylate cyclase activating polypeptide 1 (pituitary) |
| −12.02 | ADH1C | NM_000669 | alcohol dehydrogenase 1C (class I), gamma polypeptide |
| −22.71 | AGT | NM_000029 | angiotensinogen (serine (or cysteine) proteinase inhibitor, |
| | | | clade A (alpha-1 antiproteinase, antitrypsin), member 8) |
| −2.71 | ALG5 | NM_013338 | asparagine-linked glycosylation 5 homolog (yeast, dolichyl- |
| | | | phosphate beta-glucosyltransferase) |
| −1.92 | ANAPC4 | NM_013367 | anaphase promoting complex subunit 4 |
| −13.15 | APOA1 | NM_000039 | apolipoprotein A-I |
| −2.29 | ARL6IP | NM_015161 | ADP-ribosylation factor-like 6 interacting protein |
| −2.21 | ASAH2 | AF250847 | N-acylsphingosine amidohydrolase (non-lysosomal |
| | | | ceramidase) 2 |
| −8.24 | ATP1B4 | AI659245 | ATPase, (Na+)/K+ transporting, beta 4 polypeptide |
| −9.43 | ATP2B3 | NM_021949 | ATPase, Ca++ transporting, plasma membrane 3 |
| −2.08 | BAD | NM_004322 | BCL2-antagonist of cell death |
| −2.22 | BAG2 | NM_004282 | BCL2-associated athanogene 2 |
| −23.65 | BBS2 | T26496 | Bardet-Biedl syndrome 2 |
| −2.17 | BCAP29 | NM_018844 | B-cell receptor-associated protein 29 |
| −12.57 | BGN | NM_001711 | biglycan |
| −1.91 | BIRC5 | NM_001168 | baculoviral IAP repeat-containing 5 (survivin) |
| −2.58 | BLM | NM_000057 | Bloom syndrome |
| −2.11 | C10ORF7 | NM_006023 | chromosome 10 open reading frame 7 |
| −15.52 | C3AR1 | NM_004054 | complement component 3a receptor 1 |
| −1.92 | CAMK1 | NM_003656 | calcium/calmodulin-dependent protein kinase I |
| −11.45 | CASR | BX106711 | calcium-sensing receptor (hypocalciuric hypercalcemia 1, |
| | | | severe neonatal hyperparathyroidism) |
| −12.99 | CCL23 | NM_005064 | chemokine (C—C motif) ligand 23 |
| −2.51 | CCNB2 | NM_004701 | cyclin B2 |
| −2.14 | CD164 | NM_006016 | CD164 antigen, sialomucin |
| −2.40 | CD58 | NM_001779 | CD58 antigen, (lymphocyte function-associated antigen 3) |
| −13.25 | CD5L | NM_005894 | CD5 antigen-like (scavenger receptor cysteine rich family) |
| −2.09 | CDC2 | NM_001786 | cell division cycle 2, G1 to S and G2 to M |
| −2.99 | CDC25C | NM_001790 | cell division cycle 25C |
| −8.14 | CDKL1 | NM_004196 | cyclin-dependent kinase-like 1 (CDC2-related kinase) |
| −19.89 | CENTA1 | NM_006869 | centaurin, alpha 1 |
| −2.14 | CHRNA5 | NM_000745 | cholinergic receptor, nicotinic, alpha polypeptide 5 |
| −2.38 | CKS1B | NM_001826 | CDC28 protein kinase regulatory subunit 1B |
| −54.48 | COL1A2 | NM_000089 | collagen, type I, alpha 2 |
| −2.54 | COPS3 | NM_003653 | COP9 constitutive photomorphogenic homolog subunit 3 |
| | | | (Arabidopsis) |
| −23.19 | COX6A2 | NM_005205 | cytochrome c oxidase subunit VIa polypeptide 2 |
| −2.13 | CREM | NM_001881 | cAMP responsive element modulator |
| −2.53 | CSE1L | NM_001316 | CSE1 chromosome segregation 1-like (yeast) |
| −26.26 | CSRP3 | NM_003476 | cysteine and glycine-rich protein 3 (cardiac LIM protein) |
| −20.65 | CYP19A1 | NM_000103 | cytochrome P450, family 19, subfamily A, polypeptide 1 |
| −1.88 | D8S2298E | NM_005671 | reproduction 8 |
| −1.94 | DCK | NM_000788 | deoxycytidine kinase |
| −3.14 | DCLRE1A | NM_014881 | DNA cross-link repair 1A (PSO2 homolog, S. cerevisiae) |
| −2.09 | DDX1 | NM_004939 | DEAD (Asp-Glu-Ala-Asp) box polypeptide 1 |
| −2.36 | DEK | NM_003472 | DEK oncogene (DNA binding) |
| −2.95 | DHFR | AU127142 | dihydrofolate reductase |
| −3.39 | DLEU2 | NM_006021 | deleted in lymphocytic leukemia, 2 |
| −2.15 | DNAJB11 | NM_016306 | DnaJ (Hsp40) homolog, subfamily B, member 11 |
| −2.24 | DNAJD1 | NM_013238 | DnaJ (Hsp40) homolog, subfamily D, member 1 |
| −89.97 | DSC3 | NM_001941 | desmocollin 3 |
| −9.75 | DSCR1L1 | NM_005822 | Down syndrome critical region gene 1-like 1 |
| −3.07 | DTYMK | NM_012145 | deoxythymidylate kinase (thymidylate kinase) |
| −2.18 | DUSP12 | NM_007240 | dual specificity phosphatase 12 |
| −2.08 | DUT | NM_001948 | dUTP pyrophosphatase |
| −28.71 | EGFL6 | NM_015507 | EGF-like-domain, multiple 6 |
| −15.24 | EIF2AK4 | AI630242 | eukaryotic translation initiation factor 2 alpha kinase 4 |
| −2.13 | EIF2S1 | NM_004094 | eukaryotic translation initiation factor 2, subunit 1 alpha, |
| | | | 35 kDa |
| −15.36 | EIF4EL3 | BX111619 | eukaryotic translation initiation factor 4E-like 3 |
| −6.35 | ENG | BM665467 | endoglin (Osler-Rendu-Weber syndrome 1) |
| −3.39 | ERH | NM_004450 | enhancer of rudimentary homolog (Drosophila) |
| −3.32 | FAIM | NM_018147 | Fas apoptotic inhibitory molecule |
| −2.11 | FARS1 | NM_006567 | phenylalanine-tRNA synthetase 1 (mitochondrial) |
| −17.42 | FBLN1 | NM_001996 | fibulin 1 |
| −13.00 | FBN1 | NM_000138 | fibrillin 1 (Marfan syndrome) |
| −11.62 | FCAR | NM_002000 | Fc fragment of IgA, receptor for |
| −2.87 | FEN1 | NM_004111 | flap structure-specific endonuclease 1 |
| −9.02 | FNTA | BI715309 | farnesyltransferase, CAAX box, alpha |
| −9.60 | FOXN1 | NM_003593 | forkhead box N1 |
| −10.14 | GABRA3 | NM_000808 | gamma-aminobutyric acid (GABA) A receptor, alpha 3 |
| −2.44 | GDAP1 | NM_018972 | ganglioside-induced differentiation-associated protein 1 |
| −1.96 | GGH | NM_003878 | gamma-glutamyl hydrolase (conjugase, |
| | | | folylpolygammaglutamyl hydrolase) |
| −19.73 | GIPR | NM_000164 | gastric inhibitory polypeptide receptor |
| −62.09 | GJB5 | NM_005268 | gap junction protein, beta 5 (connexin 31.1) |
| −2.51 | GLA | NM_000169 | galactosidase, alpha |
| −2.81 | GMNN | NM_015895 | geminin, DNA replication inhibitor |
| −18.16 | GNAL | BX116836 | guanine nucleotide binding protein (G protein), alpha |
| | | | activating activity polypeptide, olfactory type |
| −11.49 | GPR1 | CB992712 | G protein-coupled receptor 1 |
| −80.43 | GPR15 | NM_005290 | G protein-coupled receptor 15 |
| −13.80 | GPR24 | NM_005297 | G protein-coupled receptor 24 |
| −2.56 | GPR54 | NM_032551 | G protein-coupled receptor 54 |
| −2.31 | H2AFX | NM_002105 | H2A histone family, member X |
| −2.35 | H2AFZ | NM_002106 | H2A histone family, member Z |
| −2.63 | HAT1 | NM_003642 | histone acetyltransferase 1 |
| −1.97 | HMGB1 | NM_002128 | high-mobility group box 1 |
| −2.94 | HMMR | NM_012484 | hyaluronan-mediated motility receptor (RHAMM) |
| −7.42 | HNF4A | NM_000457 | hepatocyte nuclear factor 4, alpha |
| −1.93 | HNRPA2B1 | NM_002137 | heterogeneous nuclear ribonucleoprotein A2/B1 |
| −2.08 | HSGT1 | NM_007265 | suppressor of S. cerevisiae gcr2 |
| −13.76 | HSPB2 | NM_001541 | heat shock 27 kDa protein 2 |
| −58.56 | IBSP | NM_004967 | integrin-binding sialoprotein (bone sialoprotein, bone |
| | | | sialoprotein II) |
| −9.79 | IL13RA2 | NM_000640 | interleukin 13 receptor, alpha 2 |
| −11.23 | IL1RAP | AK095107 | interleukin 1 receptor accessory protein |
| −28.60 | IL1RL1 | NM_003856 | interleukin 1 receptor-like 1 |
| −25.20 | IL7R | NM_002185 | interleukin 7 receptor |
| −10.64 | ITGA2B | NM_000419 | integrin, alpha 2b (platelet glycoprotein IIb of IIb/IIIa |
| | | | complex, antigen CD41B) |
| −27.91 | ITGA9 | BF959890 | integrin, alpha 9 |
| −2.12 | ITGAE | NM_002208 | integrin, alpha E (antigen CD103, human mucosal |
| | | | lymphocyte antigen 1; alpha polypeptide) |
| −2.73 | ITGB3BP | NM_014288 | integrin beta 3 binding protein (beta3-endonexin) |
| −15.06 | ITSN1 | NM_003024 | intersectin 1 (SH3 domain protein) |
| −15.93 | KCNJ12 | NM_021012 | potassium inwardly-rectifying channel, subfamily J, |
| | | | member 12 |
| −6.83 | KCNJ15 | NM_002243 | potassium inwardly-rectifying channel, subfamily J, |
| | | | member 15 |
| −24.13 | KCNQ2 | NM_004518 | potassium voltage-gated channel, KQT-like subfamily, |
| | | | member 2 |
| −7.23 | KIAA0089 | NM_015141 | KIAA0089 protein |
| −2.52 | KIF2C | NM_006845 | kinesin family member 2C |
| −11.04 | KIF5A | AL118561 | kinesin family member 5A |
| −24.24 | KLRG1 | NM_005810 | killer cell lectin-like receptor subfamily G, member 1 |
| −3.34 | KRT13 | NM_002274 | keratin 13 |
| −11.85 | LCP2 | NM_005565 | lymphocyte cytosolic protein 2 (SH2 domain containing |
| | | | leukocyte protein of 76 kDa) |
| −57.61 | LIMS2 | NM_017980 | LIM and senescent cell antigen-like domains 2 |
| −87.48 | LNX | AL565198 | ligand of numb-protein X |
| −11.94 | LPAAT-E | NM_018361 | acid acyltransferase-epsilon |
| −2.40 | LPAAT-E | NM_018361 | acid acyltransferase-epsilon |
| −15.01 | LRP1B | NM_018557 | low density lipoprotein-related protein 1B (deleted in |
| | | | tumors) |
| −12.57 | LTB | NM_002341 | lymphotoxin beta (TNF superfamily, member 3) |
| −3.14 | MAD2L1 | NM_002358 | MAD2 mitotic arrest deficient-like 1 (yeast) |
| −12.89 | MAP6 | AB058781 | microtubule-associated protein 6 |
| −2.14 | MAPK13 | NM_002754 | mitogen-activated protein kinase 13 |
| −26.53 | MAPT | BM714794 | microtubule-associated protein tau |
| −1.89 | MAZ | NM_002383 | MYC-associated zinc finger protein (purine-binding |
| | | | transcription factor) |
| −1.97 | MCM6 | NM_005915 | MCM6 minichromosome maintenance deficient 6 (MIS5 |
| | | | homolog, S. pombe) (S. cerevisiae) |
| −2.69 | MCM7 | NM_005916 | MCM7 minichromosome maintenance deficient 7 (S. cerevisiae) |
| −2.39 | MEA | NM_014623 | male-enhanced antigen |
| −10.83 | MGAT4A | AI364966 | mannosyl (alpha-1,3-)-glycoprotein beta-1,4-N- |
| | | | acetylglucosaminyltransferase, isoenzyme A |
| −2.20 | MIS12 | NM_024039 | homolog of yeast Mis12 |
| −2.04 | MPZL1 | NM_003953 | myelin protein zero-like 1 |
| −2.02 | MRPL1 | NM_020236 | mitochondrial ribosomal protein L1 |
| −2.97 | MRPL11 | NM_016050 | mitochondrial ribosomal protein L11 |
| −2.16 | MRPL13 | NM_014078 | mitochondrial ribosomal protein L13 |
| −2.29 | MRPL23 | NM_021134 | mitochondrial ribosomal protein L23 |
| −2.20 | MRPL39 | NM_017446 | mitochondrial ribosomal protein L39 |
| −17.46 | MSLN | NM_005823 | mesothelin |
| −16.21 | MT1A | BM684446 | metallothionein 1A (functional) |
| −2.09 | MT2A | BG505162 | metallothionein 2A |
| −59.05 | MTIF2 | AI064964 | I factor (complement) |
| −2.66 | MXD3 | BQ053282 | MAX dimerization protein 3 |
| −21.22 | MYBPC2 | NM_004533 | myosin binding protein C, fast type |
| −50.59 | MYO15A | NM_016239 | myosin XVA |
| −33.67 | NCF1 | BI021745 | neutrophil cytosolic factor 1 (47 kDa, chronic |
| | | | granulomatous disease, autosomal 1) |
| −21.83 | NCF2 | NM_000433 | neutrophil cytosolic factor 2 (65 kDa, chronic |
| | | | granulomatous disease, autosomal 2) |
| −2.51 | NDUFA6 | NM_002490 | NADH dehydrogenase (ubiquinone) 1 alpha subcomplex, |
| | | | 6, 14 kDa |
| −15.58 | NDUFB3 | NM_002491 | NADH dehydrogenase (ubiquinone) 1 beta subcomplex, 3, |
| | | | 12 kDa |
| −10.52 | NDUFV3 | AW139027 | NADH dehydrogenase (ubiquinone) flavoprotein 3, 10 kDa |
| −6.89 | NEB | AI079911 | nebulin |
| −16.69 | NFATC1 | NM_006162 | nuclear factor of activated T-cells, cytoplasmic, |
| | | | calcineurin-dependent 1 |
| −2.01 | NFKBIB | NM_002503 | nuclear factor of kappa light polypeptide gene enhancer in |
| | | | B-cells inhibitor, beta |
| −2.41 | NMI | NM_004688 | N-myc (and STAT) interactor |
| −2.12 | NMU | NM_006681 | neuromedin U |
| −15.94 | NR0B1 | NM_000475 | nuclear receptor subfamily 0, group B, member 1 |
| −54.82 | NR2E1 | NM_003269 | nuclear receptor subfamily 2, group E, member 1 |
| −14.89 | NR2E3 | NM_016346 | nuclear receptor subfamily 2, group E, member 3 |
| −17.54 | NRG1 | NM_013956 | neuregulin 1 |
| −2.08 | NT5C3 | AA188573 | 5′-nucleotidase, cytosolic III |
| −17.10 | NT5E | BM994339 | 5′-nucleotidase, ecto (CD73) |
| −2.34 | NTHL1 | NM_002528 | nth endonuclease III-like 1 (E. coli) |
| −2.02 | NUCKS | NM_022731 | nuclear ubiquitous casein kinase and cyclin-dependent |
| | | | kinase substrate |
| −1.89 | NUDT1 | NM_002452 | nudix (nucleoside diphosphate linked moiety X)-type motif 1 |
| −2.68 | NUP107 | NM_020401 | nucleoporin 107 kDa |
| −16.33 | OLR1 | CD678960 | lysyl oxidase |
| −2.68 | OXCT | NM_000436 | 3-oxoacid CoA transferase 1 |
| −12.85 | PAFAH1B1 | AI674778 | platelet-activating factor acetylhydrolase, isoform Ib, alpha |
| | | | subunit 45 kDa |
| −2.25 | PAFAH1B2 | NM_002572 | platelet-activating factor acetylhydrolase, isoform Ib, beta |
| | | | subunit 30 kDa |
| −2.03 | PAICS | NM_006452 | phosphoribosylaminoimidazole carboxylase, |
| | | | phosphoribosylaminoimidazole succinocarboxamide |
| | | | synthetase |
| −19.65 | PCDH7 | NM_032457 | BH-protocadherin (brain-heart) |
| −16.89 | PCSK2 | NM_002594 | proprotein convertase subtilisin/kexin type 2 |
| −2.21 | PDCD5 | NM_004708 | programmed cell death 5 |
| −16.85 | PDE11A | NM_016953 | phosphodiesterase 11A |
| −17.53 | PECAM1 | BG739826 | platelet/endothelial cell adhesion molecule (CD31 antigen) |
| −10.55 | PFKL | AK098228 | phosphofructokinase, liver |
| −2.17 | PHAX | NM_032177 | likely ortholog of mouse phosphorylated adaptor for RNA |
| | | | export |
| −2.95 | PHF5A | NM_032758 | PHD finger protein 5A |
| −3.73 | PIR51 | NM_006479 | RAD51-interacting protein |
| −2.39 | PLK4 | NM_014264 | polo-like kinase 4 (Drosophila) |
| −8.04 | PLXNA3 | BF926082 | plexin A3 |
| −15.36 | PMPCB | AK090763 | peptidase (mitochondrial processing) beta |
| −1.88 | POLA | NM_016937 | polymerase (DNA directed), alpha |
| −2.62 | POLE2 | NM_002692 | polymerase (DNA directed), epsilon 2 (p59 subunit) |
| −2.04 | POLE4 | NM_019896 | polymerase (DNA-directed), epsilon 4 (p12 subunit) |
| −2.24 | POLR3K | NM_016310 | polymerase (RNA) III (DNA directed) polypeptide K, 12.3 kDa |
| −2.49 | PPIH | NM_006347 | peptidyl prolyl isomerase H (cyclophilin H) |
| −26.54 | PPP1R9A | AB033048 | protein phosphatase 1, regulatory (inhibitor) subunit 9A |
| −36.93 | PPP2R5A | AA496141 | protein phosphatase 2, regulatory subunit B (B56), alpha |
| | | | isoform |
| −3.78 | PRDM1 | NM_001198 | PR domain containing 1, with ZNF domain |
| −3.47 | PRIM1 | NM_000946 | primase, polypeptide 1, 49 kDa |
| −25.92 | PRLR | AA708864 | prolactin receptor |
| −22.21 | PRSS21 | NM_006799 | protease, serine, 21 (testisin) |
| −18.91 | PTPRG | BC047734 | protein tyrosine phosphatase, receptor type, G |
| −2.43 | PTTG1 | NM_004219 | pituitary tumor-transforming 1 |
| −6.00 | PXN | AW969600 | paxillin |
| −2.31 | RACGAP1 | NM_013277 | Rac GTPase activating protein 1 |
| −2.28 | RAD18 | NM_020165 | RAD18 homolog (S. cerevisiae) |
| −2.21 | RAD51 | NM_002875 | RAD51 homolog (RecA homolog, E. coli) (S. cerevisiae) |
| −2.04 | RAD54B | NM_012415 | RAD54 homolog B (S. cerevisiae) |
| −84.42 | RB1 | BI769614 | retinoblastoma 1 (including osteosarcoma) |
| −8.81 | RBBP9 | NM_006606 | retinoblastoma binding protein 9 |
| −7.09 | RCOR1 | NM_015156 | REST corepressor 1 |
| −2.02 | RFC4 | NM_002916 | replication factor C (activator 1) 4, 37 kDa |
| −2.31 | RNASEH2A | NM_006397 | ribonuclease H2, large subunit |
| −2.29 | RNF141 | NM_016422 | ring finger protein 141 |
| −11.95 | ROBO4 | NM_019055 | roundabout homolog 4, magic roundabout (Drosophila) |
| −2.54 | RPA3 | NM_002947 | replication protein A3, 14 kDa |
| −2.22 | RPC62 | NM_006468 | polymerase (RNA) III (DNA directed) polypeptide C (62 kD) |
| −39.17 | RPL4 | BF308998 | ribosomal protein L4 |
| −68.37 | RPS3 | BM693455 | ribosomal protein S3 |
| −2.38 | RQCD1 | NM_005444 | RCD1 required for cell differentiation1 homolog (S. pombe) |
| −2.43 | RYR3 | BU533957 | ryanodine receptor 3 |
| −2.14 | SARA1 | NM_020150 | SAR1a gene homolog 1 (S. cerevisiae) |
| −2.00 | SCAMP3 | NM_005698 | secretory carrier membrane protein 3 |
| −14.52 | SCNN1G | NM_001039 | sodium channel, nonvoltage-gated 1, gamma |
| −16.54 | SEMA7A | NM_003612 | sema domain, immunoglobulin domain (Ig), and GPI |
| | | | membrane anchor, (semaphorin) 7A |
| −2.84 | SFRS3 | NM_003017 | splicing factor, arginine/serine-rich 3 |
| −7.34 | SHOX | NM_000451 | short stature homeobox |
| −11.76 | SIAT7A | NM_018414 | sialyltransferase 7 ((alpha-N-acetylneuraminyl-2,3-beta- |
| | | | galactosyl-1,3)-N-acetyl galactosaminide alpha-2,6- |
| | | | sialyltransferase) A |
| −2.93 | SIN3B | AW051366 | SIN3 homolog B, transcriptional regulator (yeast) |
| −2.78 | SIVA | NM_006427 | CD27-binding (Siva) protein |
| −11.21 | SLC1A2 | NM_004171 | solute carrier family 1 (glial high affinity glutamate |
| | | | transporter), member 2 |
| −11.51 | SLC22A13 | NM_004256 | solute carrier family 22 (organic cation transporter), |
| | | | member 13 |
| −18.72 | SLC27A6 | NM_014031 | solute carrier family 27 (fatty acid transporter), member 6 |
| −2.23 | SLC35B1 | NM_005827 | solute carrier family 35, member B1 |
| −19.44 | SLC6A4 | NM_001045 | solute carrier family 6 (neurotransmitter transporter, |
| | | | serotonin), member 4 |
| −27.75 | SLC7A13 | NM_138817 | solute carrier family 7, (cationic amino acid transporter, y+ |
| | | | system) member 13 |
| −15.57 | SLC9A3 | NM_004174 | solute carrier family 9 (sodium/hydrogen exchanger), |
| | | | isoform 3 |
| −8.20 | SLC9A7 | AA279477 | solute carrier family 9 (sodium/hydrogen exchanger), |
| | | | isoform 7 |
| −17.45 | SNAI2 | NM_003068 | snail homolog 2 (Drosophila) |
| −2.31 | SNRPD3 | NM_004175 | small nuclear ribonucleoprotein D3 polypeptide 18 kDa |
| −14.75 | SPO11 | NM_012444 | SPO11 meiotic protein covalently bound to DSB-like (S. cerevisiae) |
| −6.57 | SPOCK | NM_004598 | sparc/osteonectin, cwcv and kazal-like domains |
| | | | proteoglycan (testican) |
| −16.71 | SPTB | NM_000347 | spectrin, beta, erythrocytic (includes spherocytosis, clinical |
| | | | type I) |
| −13.79 | SRY | NM_003140 | sex determining region Y |
| −2.20 | SSSCA1 | NM_006396 | Sjogren's syndrome/scleroderma autoantigen 1 |
| −1.92 | STK6 | NM_003600 | serine/threonine kinase 6 |
| −2.41 | STMN1 | NM_005563 | stathmin 1/oncoprotein 18 |
| −10.91 | SULT1E1 | NM_005420 | sulfotransferase family 1E, estrogen-preferring, member 1 |
| −293.18 | SULT4A1 | NM_014351 | sulfotransferase family 4A, member 1 |
| −2.33 | SUV39H2 | NM_024670 | suppressor of variegation 3-9 homolog 2 (Drosophila) |
| −2.23 | SYNCRIP | NM_006372 | synaptotagmin binding, cytoplasmic RNA interacting |
| | | | protein |
| −1481.77 | SYNE1 | NM_033071 | spectrin repeat containing, nuclear envelope 1 |
| −12.26 | TAC3 | NM_013251 | tachykinin 3 (neuromedin K, neurokinin beta) |
| −2.04 | TADA2L | NM_001488 | transcriptional adaptor 2 (ADA2 homolog, yeast)-like |
| −9.82 | TCP11 | NM_018679 | t-complex 11 (mouse) |
| −11.00 | TFAP2A | NM_003220 | transcription factor AP-2 alpha (activating enhancer |
| | | | binding protein 2 alpha) |
| −20.30 | TFEC | NM_012252 | transcription factor EC |
| −3.41 | THOC4 | NM_005782 | THO complex 4 |
| −2.23 | TIMM10 | NM_012456 | translocase of inner mitochondrial membrane 10 homolog |
| | | | (yeast) |
| −2.02 | TIMM23 | NM_006327 | translocase of inner mitochondrial membrane 23 homolog |
| | | | (yeast) |
| −2.34 | TK1 | NM_003258 | thymidine kinase 1, soluble |
| −2.21 | TMEM4 | NM_014255 | transmembrane protein 4 |
| −2.64 | TMPO | H57815 | thymopoietin |
| −36.20 | TNP1 | NM_003284 | transition protein 1 (during histone to protamine |
| | | | replacement) |
| −13.71 | TPSD1 | NM_012217 | tryptase delta 1 |
| −2.55 | TRA2A | BF093914 | transformer-2 alpha |
| −8.14 | TRH | NM_007117 | thyrotropin-releasing hormone |
| −2.11 | TSFM | AW603708 | Ts translation elongation factor, mitochondrial |
| −2.24 | TTK | NM_003318 | TTK protein kinase |
| −2.13 | TXNDC | NM_030755 | thioredoxin domain containing |
| −12.47 | TYRP1 | NM_000550 | tyrosinase-related protein 1 |
| −1.99 | U2AF1 | NM_006758 | U2(RNU2) small nuclear RNA auxiliary factor 1 |
| −10.12 | UBL4 | AA873769 | ubiquitin-like 4 |
| −1.92 | UMPK | NM_012474 | uridine monophosphate kinase |
| −66.26 | USP16 | NM_006447 | ubiquitin specific protease 16 |
| −12.46 | VAPA | AI671488 | VAMP (vesicle-associated membrane protein)-associated |
| | | | protein A, 33 kDa |
| −2.22 | VDAC3 | NM_005662 | voltage-dependent anion channel 3 |
| −2.79 | VRK1 | NM_003384 | vaccinia related kinase 1 |
| −2.07 | WWOX | NM_016373 | WW domain containing oxidoreductase |
| −30.18 | ZNF145 | BU607554 | zinc finger protein 145 (Kruppel-like, expressed in |
| | | | promyelocytic leukemia) |
| −2.66 | ZNF258 | NM_007167 | zinc finger protein 258 |
| −2.03 | ZNF265 | NM_005455 | zinc finger protein 265 |
| −31.24 | ZNF282 | NM_003575 | zinc finger protein 282 |
| −2.17 | ZNRD1 | NM_014596 | zinc ribbon domain containing, 1 |
| −2.31 | ZW10 | NM_004724 | ZW10 homolog, centromere/kinetochore protein |
| | | | (Drosophila) |

Analysis

Review of this listing in the context of gene ontology, reveals that Symadex™ exerts a profound, if pleiotropic effect, on mechanisms of cell aggregation and proliferation and on processes associated with invasive cellular growth, which are the hallmark of the inflammatory etiology associated with the autoimmune diseases described at the outset.

More detailed analysis of the evidence in Table 3 reveals, for example, that a significant proportion of the down regulated genes are associated with mechanisms of cell surface signaling, motility, migration and adhesion, which permit inflammatory cells to cross vascular barrier and penetrate into parenchymal layers. Those practiced in the art will recognize that these ontological relationships are described more fully in literature within databases in the public domain, from which the following information has been excerpted. Those databases include DAVID (Database for Annotation, Visualization and Integrated Discovery, from the National Institute of Allergy and Infectious Disease, http://apps1.niaid.nih.gov/david/; the sister program EASE (Expression Analysis Systematic Explorer) at the same site; and the GeneCards bioinformatics project (http://genome-www.stanford.edu/genecards/index.shtml).

For example, in the differential gene expression experiment under discussion, the down regulated genes ACTA2, ACVRL1, BGN, DSC3, ENG, FBAN1, FBLN1, HMMR, IGTA2B, ITGA2B, ITGA9, ITGAE, LIMS2, LTB, MAPT, MSLN, NMI, PCDH7, PECAM1, PRDM1, SEMA7A, VAPA all participate in the regulation of these processes via direct modulation of adhesion factors, like integrins and cadherins, or by disrupting the growth factor signals that promote their expression and the assembly of accessory proteins that further facilitate the adhesion process. Of special importance in this context is the remarkable 1500 fold down-regulation of the SYNE1 spectrin repeats. The accessory proteins in the nesprin family coded by this gene maintain nuclear organization and the structural integrity of the cellular cytoskeleton, Down-regulation of SYNE1 would be expected to impair the ability of inflammatory cells to maintain their shape and geometry during periods of invasive motility. Thus, this effect of Symadex™ on differential gene expression of the machinery for maintaining cellular conformation would yield to the collapse of those cells during trafficking, an outcome also consistent with the histopathology of Symadex's therapeutic mode of action.

The requisite processes for calcium ion and high energy phosphate generation are affected in tandem as evidenced by the down regulation of ATP1B4, ATP2B3, CAMK1, EGFL6, GPR24, IBSP, NUDT1, RAD54B, RYR3, and SLC9A7. Cell proliferation in turn is put in check through cell cycle blocking processes mediated by BIRC5, CCL23, CCNB2, CDC2, CDC25C, CKS1B, CREM, EGFL6, FCAR, IL13RA2, IL1RAP, IL1RL1, MAPK13, NRG1, PTPRG, STK6 among other such related genes. Neuromodulation via paracrine and autocrine controls is also evident in the downregulation of systems that further respond to neuroinflammatory insult, including, for example, neurotransmitter transporters associated with damaging, runaway glutamate signaling. The downregulated genes in this latter category are exemplified by ADCYAP1, GABRA3, GGH, KCNQ3, SLC1A2 (and its SLC family solute carrier homologs), and SULT4A1. This latter gene showed close to 300 fold down-regulation. It is a gene associated with heparan sulfation. Sulfated heparans constitute the “molecular velcro” that permits integrins to bind to laminins and thereby provide the linkage that permits invasive inflammatory cells to transmigrate through basal membranes into CNS parenchyma. Down regulation of a such a process would be expected to keep inflammatory cells within the confines of vascular cuffs, as has been observed to be the case in the histopathological evaluation on the Symadex™ treatment effect noted in Examples 2-8.

The integrated function of these genes affected by Symadex™ is consistent with the differential expression profile that has been observed with microarray experiments, as for example, in the work of Arnett H A et al., “Functional genomic analysis of remyelination reveals importance of inflammation in oligodendrocyte regeneration”, J. Neuroscience 23(30):9824-9832, 2003; Lindberg R L P et al., “Multiple sclerosis as a generalized CNS disease—comparative microarray analysis of normal appearing white matter and lesions in secondary progressive MS”, J. Neuroimmunology 152:154-167, 2004; and Tajouri L. et al., “Quantitative and qualitative changes in gene expression patterns characterize the activity of plaques in multiple sclerosis”, Mol. Brain Res. 119:170-183, 2003. These studies have cataloged, in a similar manner to the gene descriptions presented here, the characteristics of representative autoimmune inflammatory insults and subsequent recovery therefrom, especially in the context of autoimmune demyelinating models for which multiple sclerosis serves as a prime circumstance. Therefore, the assertion that the application of Symadex™ and its congeners in therapy for multiple sclerosis, and autoimmune diseases of similar etiology, is demonstrable in terms of the compound's molecular pharmacology.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating a patient suffering form an inflammatory disorder, comprising:
administering to said patient a therapeutically effective amount of a compound of formula (A) or a pharmaceutically acceptable salt thereof: 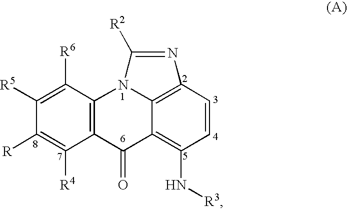
wherein:
R is —H, an optionally substituted alkyl, hydroxyl, alkoxy group, a halogen, or a group represented by the following structural formula: 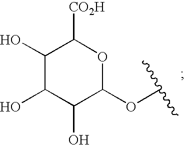
or, R and R⁵ taken together with their intervening carbon atoms form a 5, 6 or 7 member, optionally substituted, cycloalkyl or non-aromatic heterocycle;
or R and R⁴ taken together with their intervening carbon atoms form a 5, 6 or 7 member, optionally substituted, cycloalkyl or non-aromatic heterocycle; and
R² is —H, an optionally substituted C1-C10 alkyl or an optionally substituted aryl or heteroaryl;
R³ is —(CH₂)ₙ—NR^{a}R^{b}, wherein n=1-5, and R^{a} and R^{b}, each independently are hydrogen or an optionally substituted alkyl, or —NR^{a}R^{b} is an N-morpholinyl or N-pyrazinyl each optionally substituted at one or more substitutable carbons with methyl, hydroxyl, or methoxy, and wherein the N-pyrazinyl is optionally N′-substituted with C1-C4 alkyl or C1-C4 alkyl substituted with —NR^{c}R^{d}, wherein R^{c} and R^{d} are individually —H, methyl or ethyl; and
R⁴, R⁵ and R⁶, are each independently —H, —OH, a halogen or a C1-C6 alkoxy; or
R⁵ and R⁶ taken together with their intervening carbon atoms, form a 5, 6 or 7 member, optionally substitited cycloalkyl or non-aromatic heterocycle.

2. The method of claim 1, wherein the compound of formula (A) is represented by formula (I): 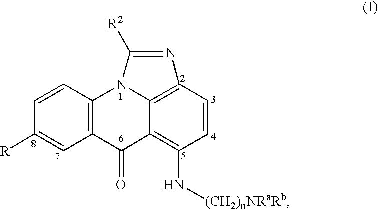
wherein
R is —OH or a C1-C6 alkoxy group;
R^{a} and R^{b}, is each independently hydrogen or an optionally substituted alkyl;
R² is —H or an C1-C6 alkyl; and
n is a whole number between 2 and 5.

3. The method of claim 2 wherein the inflammatory disorder is systemic lupus, inflammatory bowl disease, psoriasis, Crohn's disease, rheumatoid arthritis, sarcoid, Alzheimer's disease, a chronic inflammatory demyelinating neuropathy, insulin dependent diabetes mellitus, atherosclerosis, asthma, spinal cord injury or stroke.

4. The method of claim 2 wherein R is —OH or —OCH₃.

5. The method of claim 2 wherein n is 2 or 3.

6. The method of claim 2 wherein R² is a —H or a C1-C4 alkyl.

7. The method of claim 2 wherein R^{a} and R^{b} are each independently a C1-C3 alkyl.

8. The method of claim 7 wherein R^{a} and R^{b} are each independently an ethyl or a methyl.

9. The method of claim 2 wherein R^{a} and R^{b} are independently each an alkyl and optionally substituted with a C1-C4 hydroxyalkyl, an amino, a C1-C4 N-alkyl-amino or a C1-C4 N,N-dialkylamino group.

10. The method of claim 2 wherein R^{a} and R^{b} are independently each a —H or an alkyl and optionally substituted with a C1-C4 hydroxyalkyl, an amino, a C1-C4 N-alkyl-amino or a C1-C4 N,N-dialkylamino group.

11. The method of claim 9 wherein the substituents on R^{a} and R^{b} are independently hydroxyethyl, aminoethyl, N-alkylaminoethyl and N,N-dialkylaminoethyl.

12. The method of claim 2 wherein R is —OH or —OCH₃, R^{a} and R^{b} are identical and are methyl or ethyl; n is 2 or 3; R² is a hydrogen or a C1-C4 alkyl.

13. The method of claim 2 wherein the compound of formula (I) is selected from

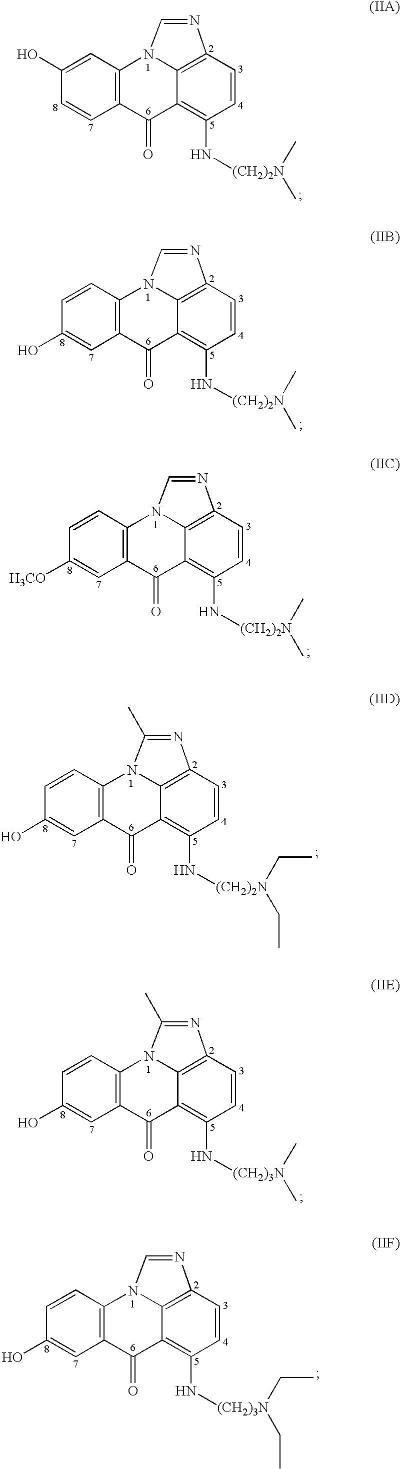

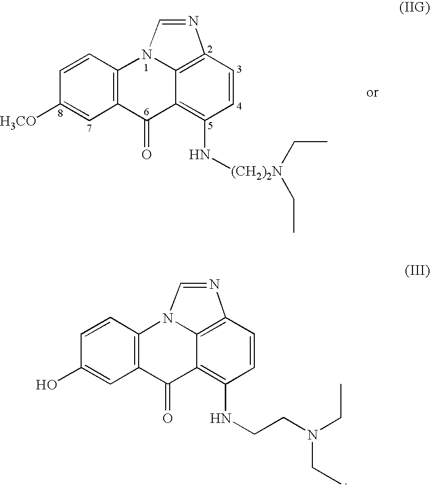

14. The method of claim 2 wherein the compound of formula (I) is a compound of formula (III):

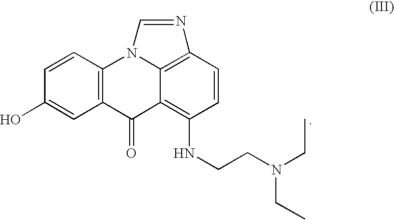

15. A method of treating a patient suffering from demyelinating condition, comprising:
administering to said patient a therapeutically effective amount of a compound of formula (A) or a pharmaceutically acceptable salt thereof: 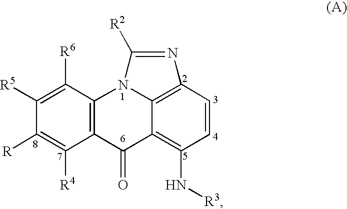
wherein:
R is —H, an optionally substituted alkyl, a hydroxyl, an alkoxy group, a halogen, a group represented by the following structural formula 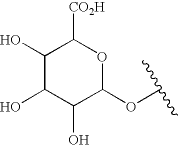
or, R and R⁵ taken together with their intervening carbon atoms form a 5, 6 or 7 member, optionally substituted, cycloalkyl or non-aromatic heterocycle;
or R and R⁴ taken together with their intervening carbon atoms form a 5, 6 or 7 member, optionally substituted, cycloalkyl or non-aromatic heterocycle;
R² is —H, an optionally substituted C1-C10 alkyl or an optionally substituted aryl or heteroaryl;
R³ is —(CH₂)ₙ—NR^{a}R^{b}, wherein n=1-5, and R^{a} and R^{b}, each independently are hydrogen or an optionally substituted alkyl, or —NR^{a}R^{b} is an N-morpholinyl or N-pyrazinyl optionally substituted at one or more substitutable carbons with methyl, hydroxyl, or methoxy group, and wherein the N-pyrazinyl is optionally N′-substituted with C1-C4 alkyl or C1-C4 alkyl substituted with —NR^{c}R^{d}, wherein R^{c} and R^{d} are individually —H, methyl or ethyl;
R⁴, R⁵ and R⁶, are each independently —H, —OH, a halogen or a C1-C6 alkoxy; or
R⁵ and R⁵ taken together with their intervening carbon atoms, form a 5, 6 or 7 member, optionally substitited cycloalkyl or non-aromatic heterocycle.

16. The method of claim 15, wherein the compound of formula (A) is represented by formula (I): 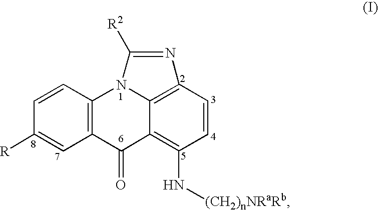
wherein
R is —OH or a C1-C6 alkoxy group;
R^{a} and R^{b}, is each independently hydrogen or an optionally substituted alkyl;
R² is —H or an C1-C6 alkyl; and
n is a whole number between 2 and 5.

17. The method of claim 16 wherein the condition is multiple sclerosis, a congenital metabolic disorder, a neuropathy with abnormal myelination, drug-induced demyelination, radiation induced demyelination, a hereditary demyelinating condition, a prion-induced demyelination, encephalitis-induced demyelination, a spinal cord injury, Alzheimer's disease or a chronic inflammatory demyelinating neuropathy.

18. The method of claim 16 wherein R is —OH or —OCH₃.

19. The method of claim 16 wherein n is 2 or 3.

20. The method of claim 16 wherein R² is a —H or a C1-C4 alkyl.

21. The method of claim 16 wherein R^{a} and R^{b} are each independently a C1-C3 alkyl.

22. The method of claim 21 wherein R^{a} and R^{b} are each independently an ethyl or a methyl.

23. The method of claim 16 wherein R^{a} and R^{b} are independently each an alkyl and optionally substituted with a C1-C4 hydroxyalkyl, an amino, a C1-C4 N-alkyl-amino or a C1-C4 N,N-dialkylamino group.

24. The method of claim 16 wherein R^{a} and R^{b} are independently each a —H or an alkyl and optionally substituted with a C1-C4 hydroxyalkyl, an amino, a C1-C4 N-alkyl-amino or a C1-C4 N,N-dialkylamino group.

25. The method of claim 23 wherein the substituents on R^{a} and R^{b} are independently hydroxyethyl, aminoethyl, N-alkylaminoethyl and N,N-dialkylaminoethyl.

26. The method of claim 16 wherein R is —OH or —OCH₃, R^{a} and R^{b} are identical and are methyl or ethyl; n is 2 or 3; R² is a hydrogen or a C1-C4 alkyl.

27. The method of claim 16 wherein the compound of formula (I) is selected from

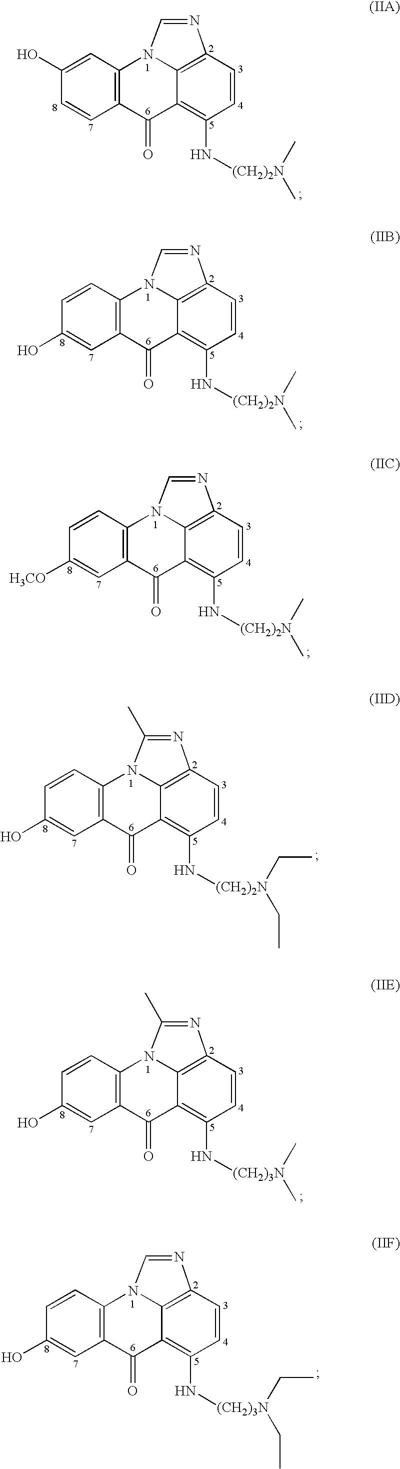

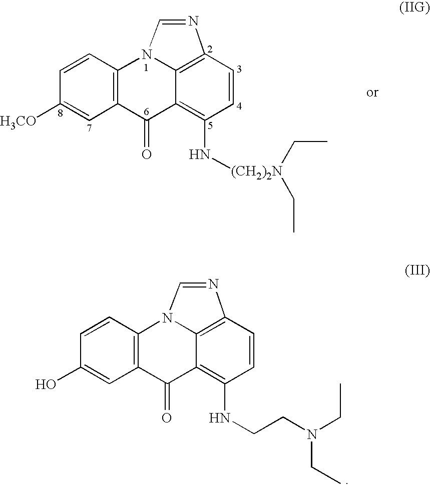

28. The method of claim 16 wherein the compound of formula (I) is a compound of formula (III):

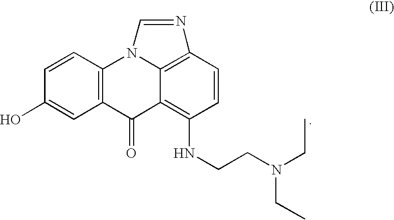

29. A method of treating a patient suffering from multiple sclerosis, comprising:
administering to said patient a therapeutically effective amount of a compound of formula (A) or a pharmaceutically acceptable salt thereof: 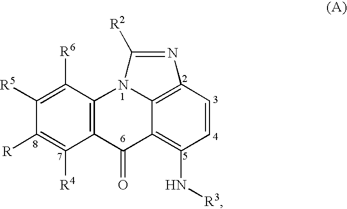
wherein:
R is —H, an optionally substituted alkyl, a hydroxyl, an alkoxy group, a halogen, a group represented by the following structural formula 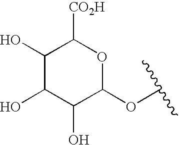
or, R and R⁵ taken together with their intervening carbon atoms form a 5, 6 or 7 member, optionally substituted, cycloalkyl or non-aromatic heterocycle;
or R and R⁴ taken together with their intervening carbon atoms form a 5, 6 or 7 member, optionally substituted, cycloalkyl or non-aromatic heterocycle;
R² is —H, an optionally substituted C1-C10 alkyl or an optionally substituted aryl or heteroaryl;
R³ is —(CH₂)ₙ—NR^{a}R^{b}, wherein n=1-5, and R^{a} and R^{b}, each independently are hydrogen or an optionally substituted alkyl, or —NR^{a}R^{b} is an N-morpholinyl or N-pyrazinyl optionally substituted at one or more substitutable carbons with methyl, hydroxyl, or methoxy group, and wherein the N-pyrazinyl is optionally N′-substituted with C1-C4 alkyl or C1-C4 alkyl substituted with —NR^{c}R^{d}, wherein R^{c} and R^{d} are individually —H, methyl or ethyl;
R⁴, R⁵ and R⁶, are each independently —H, —OH, a halogen or a C1-C6 alkoxy; or
R⁵ and R⁵ taken together with their intervening carbon atoms, form a 5, 6 or 7 member, optionally substitited cycloalkyl or non-aromatic heterocycle.

30. The method of claim 29, wherein the compound of formula (A) is represented by formula (III):

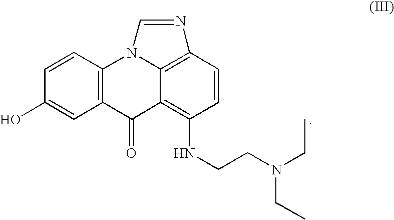

31. A method of promoting remyelination of nerve cells in a patient in need thereof, comprising administering to the patient a therapeutically effective amount of a compound of formula (A) or a pharmaceutically acceptable salt thereof: 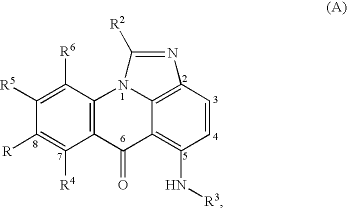
wherein:
R is —H, an optionally substituted alkyl, a hydroxyl, an alkoxy group, a halogen, a group represented by the following structural formula 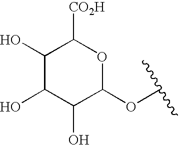
or, R and R⁵ taken together with their intervening carbon atoms form a 5, 6 or 7 member, optionally substituted, cycloalkyl or non-aromatic heterocycle;
or R and R⁴ taken together with their intervening carbon atoms form a 5, 6 or 7 member, optionally substituted, cycloalkyl or non-aromatic heterocycle;
R² is —H, an optionally substituted C1-C10 alkyl or an optionally substituted aryl or heteroaryl;
R³ is —(CH₂)ₙ—NR^{a}R^{b}, wherein n=1-5, and R^{a} and R^{b}, each independently are hydrogen or an optionally substituted alkyl, or —NR^{a}R^{b} is an N-morpholinyl or N-pyrazinyl optionally substituted at one or more substitutable carbons with methyl, hydroxyl, or methoxy group, and wherein the N-pyrazinyl is optionally N′-substituted with C1-C4 alkyl or C1-C4 alkyl substituted with —NR^{c}R^{d}, wherein R^{c} and R^{d} are individually —H, methyl or ethyl;
R⁴, R⁵ and R⁶, are each independently —H, —OH, a halogen or a C1-C6 alkoxy; or
R⁵ and R⁵ taken together with their intervening carbon atoms, form a 5, 6 or 7 member, optionally substitited cycloalkyl or non-aromatic heterocycle.

32. The method of claim 31, wherein the compound of formula (A) is represented by formula (I): 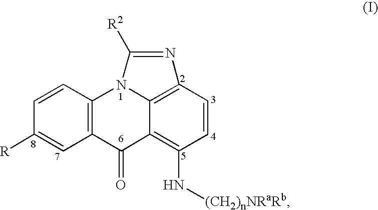
wherein
R is —OH or a C1-C6 alkoxy group;
R^{a} and R^{b}, is each independently hydrogen or an optionally substituted alkyl;
R² is —H or an C1-C6 alkyl; and
n is a whole number between 2 and 5.

33. The method of claim 32 wherein R is —OH or —OCH₃.

34. The method of claim 32 wherein n is 2 or 3.

35. The method of claim 32 wherein R² is a —H or a C1-C4 alkyl.

36. The method of claim 32 wherein R^{a} and R^{b} are each independently a C1-C3 alkyl.

37. The method of claim 36 wherein R^{a} and R^{b} are each independently an ethyl or a methyl.

38. The method of claim 32 wherein R^{a} and R^{b} are independently each an alkyl and optionally substituted with a C1-C4 hydroxyalkyl, an amino, a C1-C4 N-alkyl-amino or a C1-C4 N,N-dialkylamino group.

39. The method of claim 32 wherein R^{a} and R^{b} are independently each a —H or an alkyl and optionally substituted with a C1-C4 hydroxyalkyl, an amino, a C1-C4 N-alkyl-amino or a C1-C4 N,N-dialkylamino group.

40. The method of claim 32 wherein the substituents on R^{a} and R^{b} are independently hydroxyethyl, aminoethyl, N-alkylaminoethyl and N,N-dialkylaminoethyl.

41. The method of claim 32 wherein R is —OH or —OCH₃, R^{a} and R^{b} are identical and are methyl or ethyl; n is 2 or 3; R² is a hydrogen or a C1-C4 alkyl.

42. The method of claim 32 wherein the compound of formula (I) is selected from

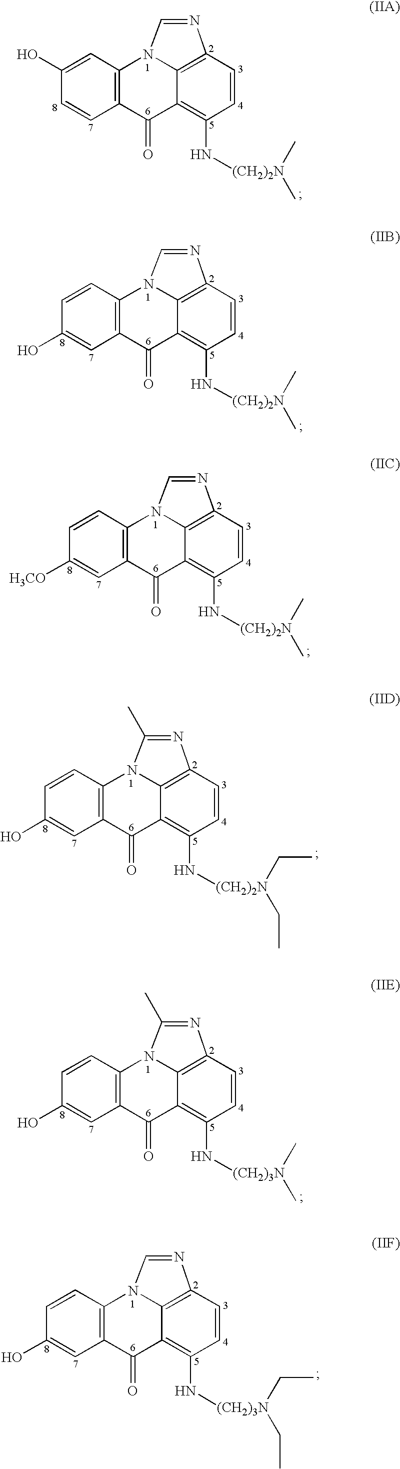

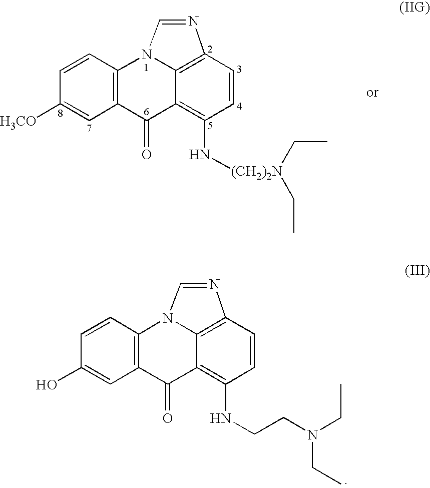

43. The method of claim 32 wherein the compound of formula (I) is a compound of formula (III):

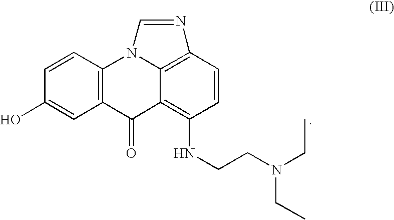

44. The method of claim 32, wherein the patient is a human.

45. The method of claim 44, wherein the human suffers from a condition which demyelinates cells, and wherein the condition is multiple sclerosis, a congenital metabolic disorder, a neuropathy with abnormal myelination, drug induced demyelination, radiation induced demyelination, a hereditary demyelinating condition, a prion induced demyelinating condition, an encephalitis induced demyelination, a spinal cord injury, Alzheimer's disease or a chronic inflammatory demyelinating neuropathy.

46. The method of claim 44, wherein the human suffers from multiple sclerosis.

47. The method of claim 32, wherein the compound is administered parenterally.

48. The method of claim 32, wherein the compound is administered chronically to the patient in need thereof.

49. The method of claim 48, wherein the chronic administration of the compound is weekly or monthly over a period of at least one year.

50. The method of claim 32, wherein an anti-inflammatory agent is co-administered with the compound to the patient.

51. The method of claim 32, wherein an EGFR inhibitor is co-administered with the compound to the patient.

52. The method of claim 32, wherein a VEGFR inhibitor is co-administered with the compound to the patient.

53. The method of claim 32, wherein a FGFR inhibitor is co-administered with the compound to the patient.

54. The method of claim 32, wherein an inhibitor of T cell homing, extravastion or transmigration is co-administered with the compound to the patient.

55. The method of claim 32, wherein a VLA4 inhibitor is co-administered with the compound to the patient.

56. The method of claim 32, wherein an interferon is co-administered with the compound to the patient.

57. The method of claim 32, wherein a chemotherapeutic agent is co-administered with the compound to the patient.

58. The method of claim 32, wherein an immunotherapeutic agent is co-administered with the compound to the patient.

59. The method of claim 50, wherein the anti-inflammatory agent is adrenocorticotropic hormone, a corticosteroid, an interferon, glatiramer acetate, or a non-steroidal anti-inflammatory drug.

60. The method of claim 59, wherein the interferon is interferon beta-1b or interferon beta-1a.

61. The method of claim 59, wherein the corticosteroid is prednisone, methylprednisolone, dexamethasone cortisol, cortisone, fludrocortisone, prednisolone, 6α-methylprednisolone, triamcinolone, or betamethasone.

62. The method of claim 59, wherein the corticosteroid is prednisone.

63. The method according to claim 59, wherein the non-steroidal anti-inflammatory drug is aspirin, a sodium salicylate, choline magnesium trisalicylate, salsalate, diflunisal, sulfasalazine, olsalazine, a para-aminophenol derivatives, an indole, an indene acetic acid, a heteroaryl acetic acid, an anthranilic acid, an enolic acid, an alkanones, a diaryl-substituted furanone, a diaryl-substituted pyrazoles, an indole acetic acids, or a sulfonanilide.

64. The method of claim 32, wherein the compound is administered orally, intravenously or subcutaneously.

65. The method according to claim 64, wherein the compound is administered intravenously to a patient, and wherein the administration results in an effective blood level of the compound in the patient of more than or equal to 10 ng/ml.

66. The method according to claim 64, wherein the compound is administered intravenously in an amount of 20 μg to about 500 μg per kilogram body weight of the patient.

67. A composition comprising a therapeutically effective amount of a compound of formula (A) below, or pharmaceutically acceptable salt thereof, and an anti-inflammatory agent: 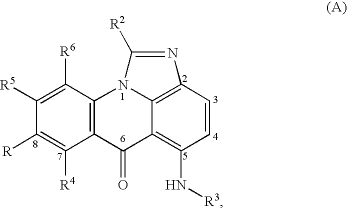
wherein:
R is —H, an optionally substituted alkyl, a hydroxyl, an alkoxy group, a halogen, a group represented by the following structural formula 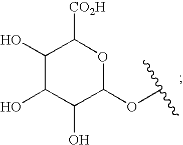
or, R and R⁵ taken together with their intervening carbon atoms form a 5, 6 or 7 member, optionally substituted, cycloalkyl or non-aromatic heterocycle;
or R and R⁴ taken together with their intervening carbon atoms form a 5, 6 or 7 member, optionally substituted, cycloalkyl or non-aromatic heterocycle;
R² is —H, an optionally substituted C1-C10 alkyl or an optionally substituted aryl or heteroaryl;
R³ is —(CH₂)ₙ—NR^{a}R^{b}, wherein n=1-5, and R^{a} and R^{b}, each independently are hydrogen or an optionally substituted alkyl, or —NR^{a}R^{b} is an N-morpholinyl or N-pyrazinyl optionally substituted at one or more substitutable carbons with methyl, hydroxyl, or methoxy group, and wherein the N-pyrazinyl is optionally N′-substituted with C1-C4 alkyl or C1-C4 alkyl substituted with —NR^{c}R^{d}, wherein R^{c} and R^{d} are individually —H, methyl or ethyl;
R⁴, R⁵ and R⁶, are each independently —H, —OH, a halogen or a C1-C6 alkoxy; or
R⁵ and R⁵ taken together with their intervening carbon atoms, form a 5, 6 or 7 member, optionally substitited cycloalkyl or non-aromatic heterocycle.

68. The composition of claim 67, wherein the compound of formula (A) is represented by formula (I): 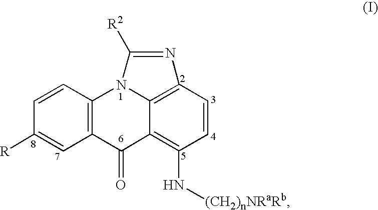
wherein
R is —OH or a C1-C6 alkoxy group;
R^{a} and R^{b}, is each independently hydrogen or an optionally substituted alkyl;
R² is —H or an C1-C6 alkyl; and
n is a whole number between 2 and 5.

69. The composition of claim 68, wherein R is —OH or —OCH₃.

70. The composition of claim 68, wherein n is 2 or 3.

71. The composition of claim 68, wherein R² is a —H or a C1-C4 alkyl.

72. The composition of claim 68, wherein R^{a} and R^{b} are each independently a C1-C3 alkyl.

73. The composition of claim 68, wherein R^{a} and R^{b} are each independently an ethyl or a methyl.

74. The method of claim 68 wherein R^{a} and R^{b} are independently each an alkyl and optionally substituted with a C1-C4 hydroxyalkyl, an amino, a C1-C4 N-alkyl-amino or a C1-C4 N,N-dialkylamino group.

75. The method of claim 68 wherein R^{a} and R^{b} are independently each a —H or an alkyl and optionally substituted with a C1-C4 hydroxyalkyl, an amino, a C1-C4 N-alkyl-amino or a C1-C4 N,N-dialkylamino group.

76. The composition of claim 75, wherein the substituents on R^{a} and R^{b} are independently hydroxyethyl, aminoethyl, N-alkylaminoethyl and N,N-dialkylaminoethyl.

77. The composition of claim 68, wherein R is —OH or —OCH₃, R^{a} and R^{b} are identical and are methyl or ethyl; n is 2 or 3; R² is a hydrogen or a C1-C4 alkyl.

78. The composition of claim 68, wherein the compound of formula (I) is selected from

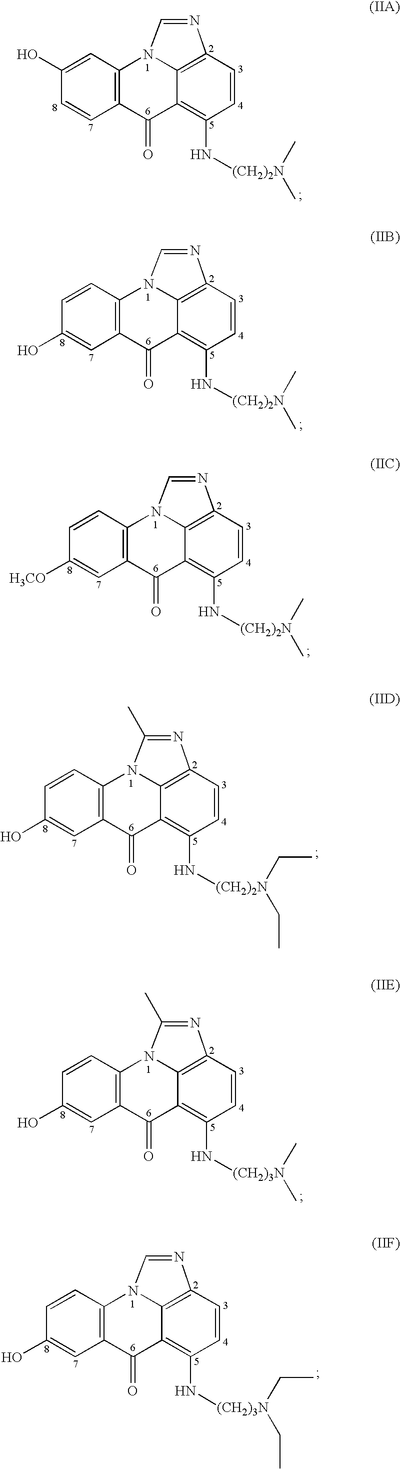

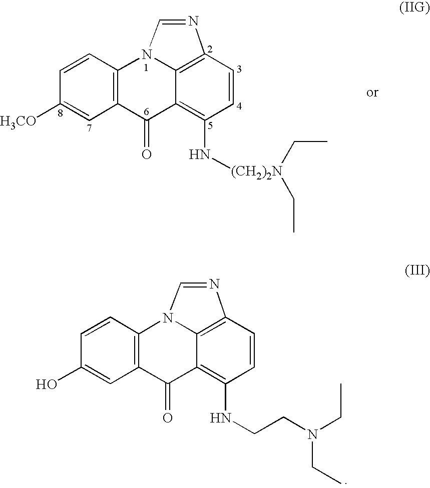

79. The composition of claim 68, wherein the compound of formula (I) is a compound of formula (III):

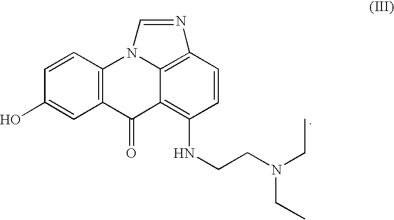

80. A method of reversing paralysis in a patient resulting from a demyelinating disease, comprising administering to the patient a compound in an amount sufficient to inhibit lymphocyte infiltration of immune cells in the spinal cord to promote remyelination of nerve cells in the spinal cord and thereby treating paralysis in said patient, wherein the compound is of formula formula (A) or a pharmaceutically acceptable salt thereof: 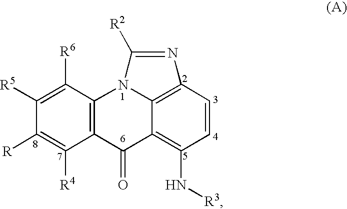
wherein:
R is —H, an optionally substituted alkyl, a hydroxyl, an alkoxy group, a halogen, a group represented by the following structural formula 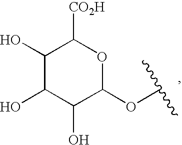
or, R and R⁵ taken together with their intervening carbon atoms form a 5, 6 or 7 member, optionally substituted, cycloalkyl or non-aromatic heterocycle;
or R and R⁴ taken together with their intervening carbon atoms form a 5, 6 or 7 member, optionally substituted, cycloalkyl or non-aromatic heterocycle;
R² is —H, an optionally substituted C1-C10 alkyl or an optionally substituted aryl or heteroaryl;
R³ is —(CH₂)ₙ—NR^{a}R^{b}, wherein n=1-5, and R^{a} and R^{b}, each independently are hydrogen or an optionally substituted alkyl, or —NR^{a}R^{b} is an N-morpholinyl or N-pyrazinyl optionally substituted at one or more substitutable carbons with methyl, hydroxyl, or methoxy group, and wherein the N-pyrazinyl is optionally N′-substituted with C1-C4 alkyl or C1-C4 alkyl substituted with —NR^{c}R^{d}, wherein R^{c} and R^{d} are individually —H, methyl or ethyl;
R⁴, R⁵ and R⁶, are each independently —H, —OH, a halogen or a C1-C6 alkoxy; or
R⁵ and R⁵ taken together with their intervening carbon atoms, form a 5, 6 or 7 member, optionally substitited cycloalkyl or non-aromatic heterocycle.

81. The method of claim 80, wherein the compound of formula (A) is represented by formula (I): 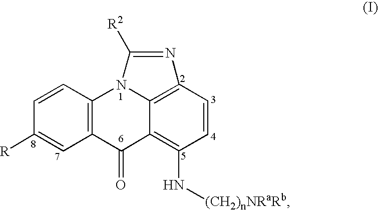
wherein
R is —OH or a C1-C6 alkoxy group;
R^{a} and R^{b}, is each independently hydrogen or an optionally substituted alkyl;
R² is —H or an C1-C6 alkyl; and
n is a whole number between 2 and 5.

82. The method of claim 81, wherein R is —OH or —OCH₃.

83. The method of claim 81, wherein n is 2 or 3.

84. The method of claim 81, wherein R² is a —H or a C1-C4 alkyl.

85. The method of claim 81, wherein R^{a} and R^{b} are each independently a C1-C3 alkyl.

86. The method of claim 81, wherein R^{a} and R^{b} are each independently an ethyl or a methyl.

87. The method of claim 81 wherein R^{a} and R^{b} are independently each an alkyl and optionally substituted with a C1-C4 hydroxyalkyl, an amino, a C1-C4 N-alkyl-amino or a C1-C4 N,N-dialkylamino group.

88. The method of claim 81 wherein R^{a} and R^{b} are independently each a —H or an alkyl and optionally substituted with a C1-C4 hydroxyalkyl, an amino, a C1-C4 N-alkyl-amino or a C1-C4 N,N-dialkylamino group.

89. The method of claim 87, wherein the substituents on R^{a} and R^{b} are independently hydroxyethyl, aminoethyl, N-alkylaminoethyl and N,N-dialkylaminoethyl.

90. The method of claim 81, wherein R is —OH or —OCH₃, R^{a} and R^{b} are identical and are methyl or ethyl; n is 2 or 3; R² is a hydrogen or a C1-C4 alkyl.

91. The method of claim 81, wherein the compound of formula (I) is selected from

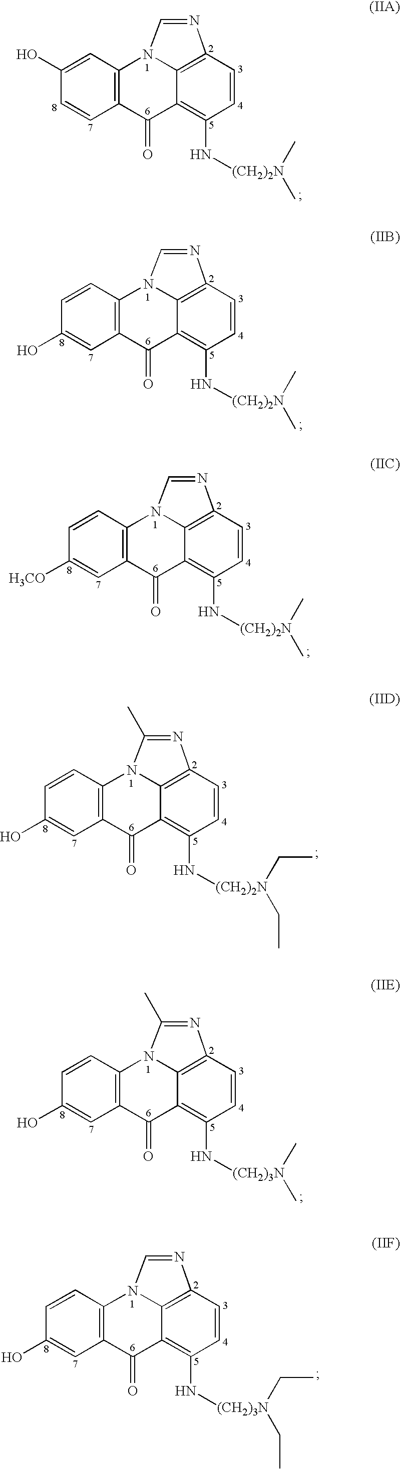

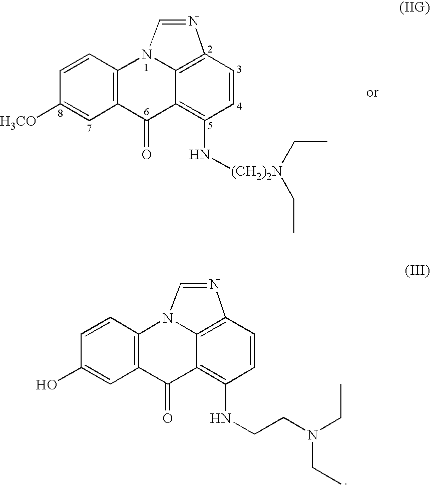

92. The method of claim 81, wherein the compound of formula (I) is a compound of formula (III):

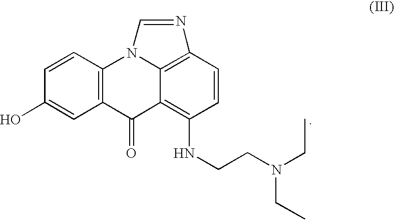

93. The method of any of claim 81, further comprising co-administering an immunosuppressant.

94. The method of claim 93, wherein the immunosuppressant is adrenocorticotropic hormone, a corticosteroid, or an interferon.

95. The method of claim 94, wherein the interferon is interferon beta-1b or interferon beta-1a.

96. The method of claim 94, wherein the corticosteroid is prednisone, methylprednisolone, dexamethosone cortisol, cortisone, fludrocortisone, prednisolone, 6α-methylprednisolone, triamcinolone, or betamethasone.
